(19) United States
(12) Patent Application Publication
(10) Pub. No.: US 2008/0255149 A1
(43) Pub. Date: Oct. 16, 2008

(54) Carboxyamine Compounds and Methods of Use Thereof

(75) Inventors: Markus Rolf Dobler, Arlington, MA (US); Jonathan E. Grob, Sharon, MA (US); Arup Patnaik, Somerville, MA (US); Branko Radetich, Boston, MA (US); Micheal Shultz, Stow, MA (US); Yanyi Zhu, Acton, MA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG

(21) Appl. No.: 12/088,367

(22) PCT Filed: Sep. 25, 2006
(86) PCT No.: PCT/US2006/037358

§ 371 Date: Mar. 27, 2008

Related U.S. Application Data
(60) Provisional application No. 60/720,900, filed on Sep. 27, 2005.
(60) Provisional application No. 60/754,960, filed on Dec. 28, 2005.

(51) Int. Cl. A61K 31/4035; C07D 209/44; C07C 271/06; C07D 211/70; A61K 31/435; A61K 31/445; A61K 31/496; A61P 35/02; A61P 17/06; A61P 19/02; A61P 35/00; A61K 31/438; A61K 31/44; A61K 31/235; C07D 211/10; C07D 241/04

(52) U.S. Cl. 514/255.01; 548/470; 560/24; 546/348; 546/184; 546/17; 544/391; 514/416; 514/533; 514/277; 514/315; 514/278

(57) ABSTRACT

The invention relates to the use of carboxyamine compounds and salts thereof in the treatment of HDAC dependent diseases and for the manufacture of pharmaceutical preparations for the treatment of said diseases.

### FIELD OF USE

The present invention relates to carboxyamine compositions. The invention also provides methods of use for modulating activity of a histone deacetylase.

### BACKGROUND

Reversible acetylation of histones is a major regulator of gene expression that acts by altering accessibility of transcription factors to DNA. In normal cells, histone deacetylase (“HDAC”) and histone acetyltransferase together control the level of acetylation of histones to regulate active and inactive regions of a chromosome. Acetylation of lysine residues of histone proteins induces conformational changes by destabilizing nucleosomes and allowing transcription factors access to recognition sequences in DNA. Deacetylation of histones by activity of one or more HDACs seals the chromosomal packing, leading to repression of transcription. Inhibition of HDAC results in the accumulation of hyperacetylated histones, which results in a variety of cellular responses.

Inhibitors of HDAC have been studied for their therapeutic effects on cancer cells and in other proliferative diseases. For example, butyric acid and its derivatives, including sodium phenylbutyrate, have been reported to induce apoptosis in vitro in human colon carcinoma, leukemia and retinoblastoma cell lines. However, butyric acid and its derivatives are not useful pharmacological agents because they tend to be metabolized rapidly and have a very short half-life in vivo. Other inhibitors of HDAC that have been widely studied for their anti-proliferative activities are trichostatin A and trapoxin. Trichostatin A is an antifungal and antibiotic and is a reversible inhibitor of mammalian HDAC. Trapoxin is a cyclic tetrapeptide, which is an irreversible inhibitor of mammalian HDAC. Although trichostatin and trapoxin have been studied for their anti-cancer activities, the in vivo instability of the compounds makes them less suitable as anti-cancer drugs. Thalidomide has recently been reported to target HDAC, but thalidomide has pleiotropic effects and is an immunomodulatory with multiple side effects including teratogenicity.

Certain inhibitors of HDAC are compounds containing a hydroxamate group, i.e., a nitrogen atom bonded to a hydroxyl group and to a carbonyl group. HDAC is a metallo-enzyme wherein the active site includes a pocket with a zinc molecule. Hydroxamate groups interact with metal ions such as zinc in active sites of enzymes to disrupt the functionality of the enzyme. However, a hydroxamate reacts in general with many different metal ions. Therefore, a therapeutic compound containing a hydroxamate often has undesirable side effects due to lack of specificity. There remains a need for an active compound that is suitable for treating proliferative diseases, including cancerous tumors, that is stable, highly efficacious, and specific with few side effects.

### SUMMARY OF THE INVENTION

The present invention provides in certain embodiments, efficacious compounds that are useful as pharmaceutical agents. In general, a compound of the present invention is shown in formula I:

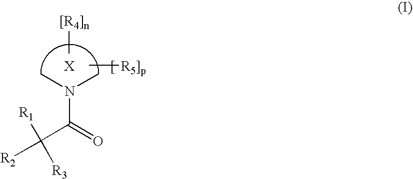

in which:
R₁ can be H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
X is a selected from a C₃-C₆ cycloalkyl, C₃-C₆ cycloalkenyl, aryl, C₃-C₆ heterocycloalkyl, C₃-C₆ heteroaryl, and a polyheterocycle, any of which may be further heterosubstituted, wherein specific examples of polyheterocycles may be selected from

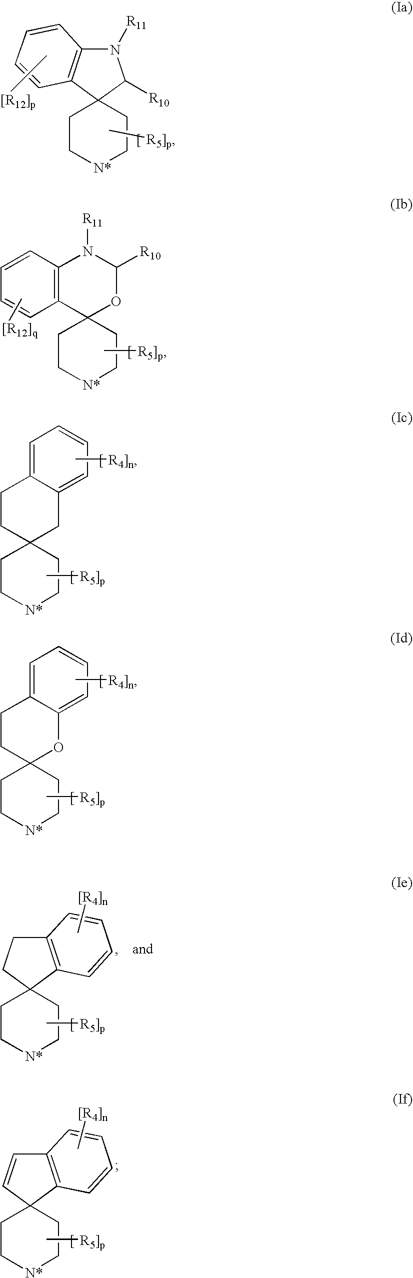

R₄ is present at n occurrences, n is an integer from 0 to 4, and R₄ is the same or different and independently selected from H, lower alkyl, hetero-substituted lower alkyl, alkylaryl, hetero-substituted alkylaryl, lower alkoxy, C₃-C₆ cycloalkyl, aryl, C₃-C₆ heterocycloalkyl, C₃-C₆ heteroaryl, N—(R₁₃)₂, S—R₁₃, O—R₁₃, or a mixed aryl and non-aryl polyheterocycle ring (such as, e.g., benzhydryl or 9H-fluorenyl), any of which may be further substituted by R₈;
R₅ is present at p occurrences, p is an integer from 0 to 4, and R₅ is the same or different and independently selected from H, O, halo, lower alkoxy, and a straight or branched lower alkyl or hetero-substituted lower alkyl;
R₆ is H or a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₈ is selected from one or more of H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy; any of which may be further substituted by R₉;
R₉ is selected from one or more of H, halo, COOH, lower alkyl, hetero-substituted lower alkyl, aryl, and lower alkoxy;
R₁₀ and R₁₁ are selected from H, O, halo, lower alkyl, hetero-substituted lower alkyl, and lower alkoxy; and
R₁₂ is present at q occurrences wherein q is an integer from 0 to 4, and R₁₂ is the same or different and independently selected from are selected from H, O, halo, lower alkyl, hetero-substituted lower alkyl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
   or a pharmaceutically acceptable salt thereof.

In another embodiment, a compound of the present invention has formula II:

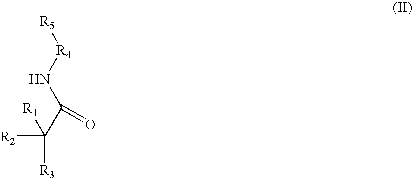

in which:
R₁ can be H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
R₄ is selected from C₃-C₆ cycloalkyl, aryl, C₃-C₆ heterocycloalkyl, C₃-C₆ heteroaryl, or a mixed aryl and non-aryl polyheterocycle ring, any of which may be further substituted by R₇;
R₅ is present at p occurrences, p is an integer from 0 to 3, and R₅ is the same or different and independently selected from H, O, halo, lower alkoxy, and a straight or branched lower alkyl or hetero-substituted lower alkyl;
R₆ is H or a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R;
R₈ is selected from H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy, N—(R₁₃)₂, S—R₁₃, O—R₁₃; any of which may be further substituted by R₉;
R₉ is selected from H, halo, lower alkyl, hetero-substituted lower alkyl, aryl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
   or a pharmaceutically acceptable salt of any of these compounds.

In other embodiments, the invention provides compounds in which at least one of R₁, R₂, or R₃ is selected from hydrogen. In related embodiments, the invention provides compounds in which at least one of R₁, R₂, or R₃ is selected from the group of NHR₆ or NH₂. In a preferred embodiment, the invention provides compounds in which R₁ is NH₂, and R₂ is H.

Unless specifically stated, reference to any of the R groups in any of the provided formulations does not infer chirality or stereospecificity.

In certain embodiments, a compound of the present invention is further characterized as modulator of a histone deacetylase (“HDAC”), including a mammalian HDAC, and especially including a human HDAC polypeptide. In a preferred embodiment, the aminoamine compound of the invention is a HDAC inhibitor. A preferred HDAC inhibitor is a non-hydroxamate, non-thio containing compound of the invention.

In preferred embodiments, the invention provides a method for treating a HDAC dependent disease. The method includes administering to a mammal with a HDAC dependent disease, a preferred compound of the present invention. In a related embodiment, the protein HDAC of the present method is selected from the group of HDAC1, HDAC2, HDAC3, HDAC4, HDAC5, HDAC6, HDAC7, HDAC8, HDAC9, HDAC10 and HDAC11. In a further embodiment, the protein HDAC of the method is selected from the group of HDAC1, HDAC2, HDAC6, and HDAC8.

In other embodiments, the present invention provides a method for inhibiting a histone deacetylase. The method includes contacting a cell with any of the compounds of the present invention. In a related embodiment, the method further provides that the compound is present in an amount effective to produce a concentration sufficient to selectively inhibit the acetylation of a histone in the cell.

In other embodiments, the present invention provides a use of any of the compounds of the invention for manufacture of a medicament to treat a proliferative or hyperproliferative disease.

In other embodiments, the invention provides a method of manufacture of a medicament, including formulating any of the compounds of the present invention for treatment of a subject.

In embodiments related to these uses and methods, the disease includes a proliferative disease, which includes a benign or malignant tumor, a carcinoma of the brain, kidney, liver, adrenal gland, bladder, breast, stomach (for example gastric tumors), ovaries, esophagus, colon, rectum, prostate, pancreas, lung, vagina, thyroid, sarcoma, glioblastomas, multiple myeloma or gastrointestinal cancer, for example, colon carcinoma or colorectal adenoma, or a tumor of the neck and head, an epidermal hyperproliferation, for example, psoriasis, prostate hyperplasia, a neoplasia, including a neoplasia of epithelial character, including mammary carcinoma, or a leukemia.

In still another related embodiment, the disease to be treated by the uses and methods of the present invention is selected from triggering by persistent proliferative conditions such as angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and treating age spots and contact dermatitis.

In embodiments related to these uses and methods, the disease includes a hyperproliferative disease, which includes leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds.

In other embodiments, the invention provides a kit including any of the compounds of the present invention. In a related embodiment, the kit further includes a pharmaceutically acceptable carrier or excipient of any of these compounds. In another related embodiment, the compounds of the invention, present in the kit, are in a unit dose. In still another related embodiment, the kit further includes instructions for use in administering to a subject.

As is evident to those skilled in the art, many of the compounds of the present invention contain asymmetric carbon atoms. It should be understood, therefore, that all individual stereoisomers of the provided formulas are contemplated as being included within the scope of this invention.

The compounds of the present invention are suitable as active agents in pharmaceutical compositions that are efficacious particularly for treating cellular proliferative ailments and/or ailments associated with misregulated gene expression. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially an anti-tumor effect, e.g., inhibition of proliferation of malignant cancer cells, benign tumor cells or other proliferative cells, or of any other HDAC dependent disease.

### DETAILED DESCRIPTION

The present invention provides aminoalkyl compounds. A function of these compounds includes, for example, inhibition of deacetylases or inhibition of histone deacetylases. The aminoalkyl compounds are suitable for treating, for example, tumors, including cancerous tumors, and cardiovascular diseases. In certain embodiments, the aminoalkyl compounds of the present invention have the following structures provided in formula I and formula II.

In certain embodiments, the present invention provides compounds having the formula I,

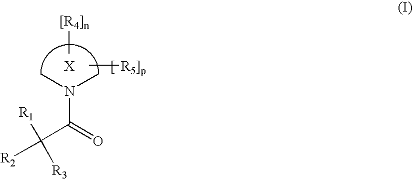

wherein:
R₁ is selected from H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
X is a selected from a C₃-C₆ cycloalkyl, C₃-C₆ cycloalkenyl, aryl, C₃-C₆ heterocycloalkyl, C₃-C₆ heteroaryl, and a polyheterocycle, any of which may be further heterosubstituted, wherein specific examples of polyheterocycles may be selected from

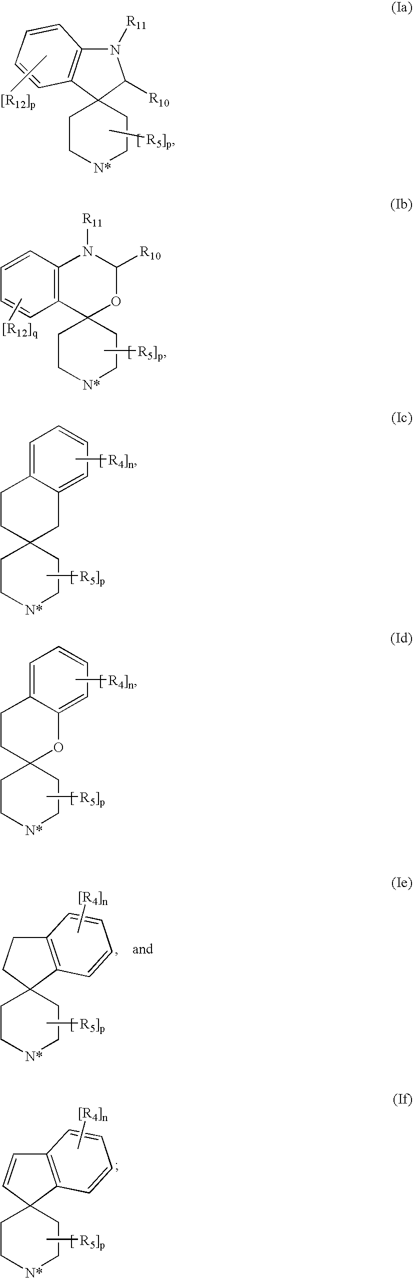

R₄ is present at n occurrences, n is an integer from 0 to 4, and R₄ is the same or different and independently selected from H, lower alkyl, hetero-substituted lower alkyl, lower alkoxy, alkylaryl, hetero-substituted alkylaryl, C₃-C₆ cycloalkyl, aryl, C₃-C₆ heterocycloalkyl, C₃-C₆ heteroaryl, N—(R₁₃)₂, S—R₁₃, O—R₁₃, or a mixed aryl and non-aryl polyheterocycle ring (such as, e.g., benzhydryl or 9H-fluorenyl), any of which may be further substituted by R₈;
R₅ is present at p occurrences, p is an integer from 0 to 4, and R₅ is the same or different and independently selected from H, O, halo, lower alkoxy, and a straight or branched lower alkyl or hetero-substituted lower alkyl;
R₆ is selected from H and a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₈ is selected from H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy, N—(R₁₃)₂, S—R₁₃, O—R₁₃; any of which may be further substituted by R₉;
R₉ is selected from one or more of H, halo, COOH, lower alkyl, hetero-substituted lower alkyl, aryl, and lower alkoxy;
R₁₀ and R₁₁ are selected from H, O, halo, lower alkyl, hetero-substituted lower alkyl, and lower alkoxy;
R₁₂ is present at q occurrences wherein q is an integer from 0 to 4, and R₁₂ is the same or different and independently selected from are selected from H, O, halo, lower alkyl, hetero-substituted lower alkyl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
   or a pharmaceutically acceptable salt thereof.

A use of the compounds of formula I can be, for example, as efficacious HDAC inhibitor compounds that are useful as pharmaceutical agents.

In alternative embodiments, the present invention provides compounds having formula II,

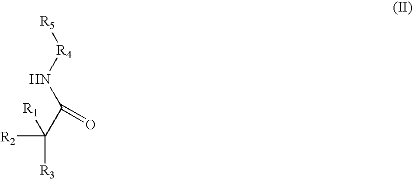

wherein
R₁ can be H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
R₄ is selected from C₃-C₆ cycloalkyl, aryl, C₃-C₆ heterocycloalkyl, C₃-C₆ heteroaryl, or a mixed aryl and non-aryl polyheterocycle ring, any of which may be further substituted by R₇;
R₅ is present at p occurrences, p is an integer from 0 to 3, and R₅ is the same or different and independently selected from H, O, halo, lower alkoxy, and a straight or branched lower alkyl or hetero-substituted lower alkyl;
R₆ is H or a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₈ is selected from H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy, N—(R₁₃)₂, S—R₁₃, O—R₁₃; any of which may be further substituted by R₉;
R₉ is selected from H, halo, lower alkyl, hetero-substituted lower alkyl, aryl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
   or a pharmaceutically acceptable salt thereof.

A use of the compounds of formula II can be, for example, as efficacious HDAC inhibitor compounds that are useful as pharmaceutical agents.

In further embodiments, the present invention provides a compound of any one of subformula III through subformula V:

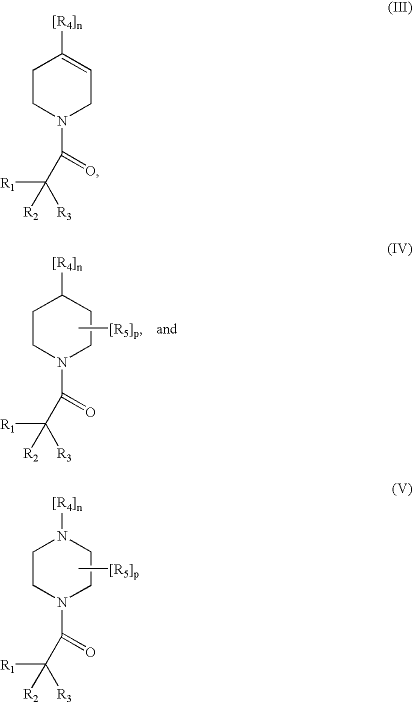

in which:
R₁ can be H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
R₄ is present at n occurrences wherein n is an integer from 0 to 4, and R₄ is the same or different and independently selected from H, lower alkyl, hetero-substituted lower alkyl, alkylaryl, hetero-substituted alkylaryl, lower alkoxy, C₃-C₆ cycloalkyl, aryl, C₃-C₆ heterocycloalkyl, C₃-C₆ heteroaryl, N—(R₁₃)₂, S—R₁₃, O—R₁₃, or a mixed aryl and non-aryl polyheterocycle ring (such as, e.g., benzhydryl or 9H-fluorenyl), any of which may be further substituted by R₈;
R₅ is present at p occurrences wherein p is an integer from 0 to 4, and R₅ is the same or different and independently selected from H, O, halo, lower alkoxy, and a straight or branched lower alkyl or hetero-substituted lower alkyl;
R₆ is H or a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₈ is selected from one or more of H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy; any of which may be further substituted by R₉; and
R₉ is selected from one or more of H, halo, COOH, lower alkyl, hetero-substituted lower alkyl, aryl, and lower alkoxy;
R₁₀ and R₁₁ are selected from H, O, halo, lower alkyl, hetero-substituted lower alkyl, and lower alkoxy; and
R₁₂ is present at q occurrences wherein q is an integer from 0 to 4, and R₁₂ is the same or different and independently selected from are selected from H, O, halo, lower alkyl, hetero-substituted lower alkyl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
   or a pharmaceutically acceptable salt of any of these.

A use of the compounds of subformula III, subformula IV or subformula V can be, for example, as efficacious HDAC inhibitor compounds that are useful as pharmaceutical agents.

In more specific embodiments, the invention provides a compound of any of subformula:

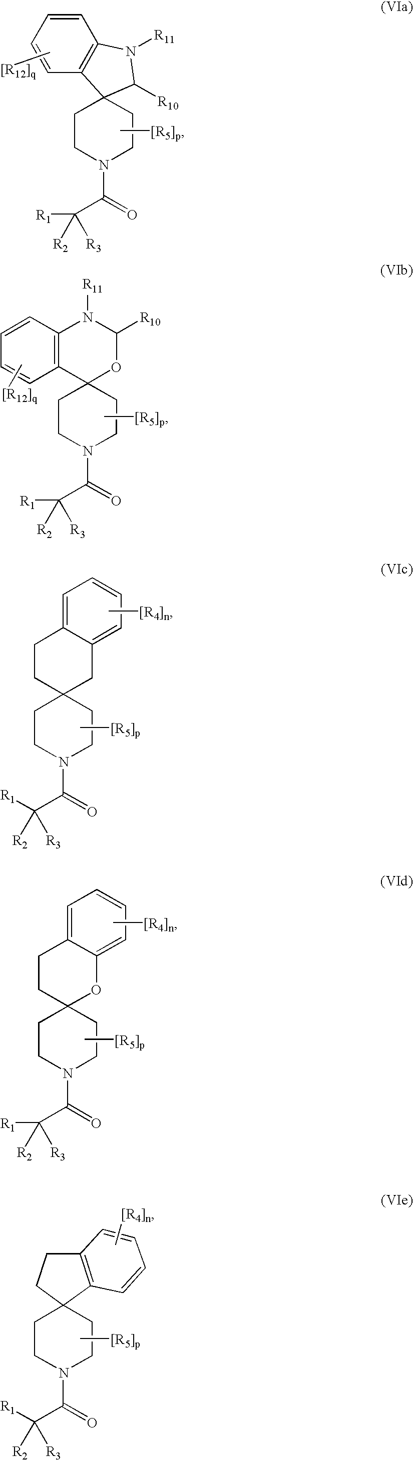

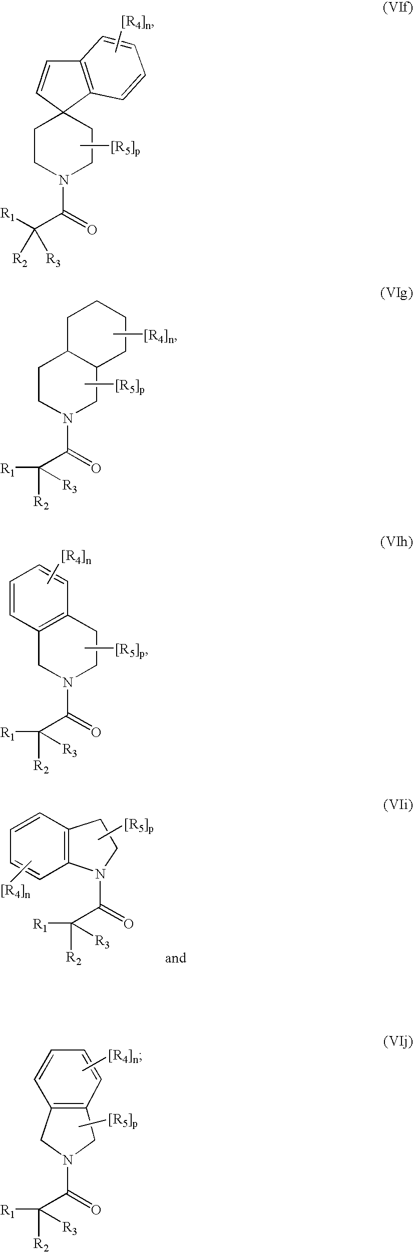

in which:
R₁ can be H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
R₄ is present at n occurrences wherein n is an integer from 0 to 4, and R₄ is the same or different and independently selected from H, lower alkyl, hetero-substituted lower alkyl, alkylaryl, hetero-substituted alkylaryl, lower alkoxy, C₃-C₆ cycloalkyl, aryl, C₃-C₆ heterocycloalkyl, C₃-C₆ heteroaryl, N—(R₁₃)₂, S—R₁₃, O—R₁₃, or a mixed aryl and non-aryl polyheterocycle ring (such as, e.g., benzhydryl or 9H-fluorenyl), any of which may be further substituted by R₈;
R₅ is present at p occurrences wherein p is an integer from 0 to 4, and R₅ is the same or different and independently selected from H, O, halo, lower alkoxy, and a straight or branched lower alkyl or hetero-substituted lower alkyl;
R₆ is H or a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₈ is selected from one or more of H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy; any of which may be further substituted by R₉;
R₉ is selected from one or more of H, halo, COOH, lower alkyl, hetero-substituted lower alkyl, aryl, and lower alkoxy;
R₁₀ and R₁₁ are selected from H, O, halo, lower allyl, hetero-substituted lower alkyl, and lower alkoxy; and
R₁₂ is present at q occurrences wherein q is an integer from 0 to 4, and R₁₂ is the same or different and independently selected from are selected from H, O, halo, lower alkyl, hetero-substituted lower alkyl, and lower alkoxy;
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
   or a pharmaceutically acceptable salt of any of these compounds.

In certain embodiments, the invention provides compounds in which X is a polyheterocycle selected from a nitrogen-substituted cycloalkyl, aryl or cycloalkaryl, any of which may be further heterosubstituted, and which for example may be selected from a C₃-C₆ cycloalkyl or partially unsaturated cycloalkyl, C₃-C₆ saturated or partially unsaturated heterocycloalkyl or heterocycloalkenyl (e.g., tetrahydro-pyridine), morpholine, C₃-C₆ heteroaryl, C₃-C₆ polyheteroaryl, C₃-C₆ non-aromatic polyheterocycle, or a fused and/or spiro polyheterocycle selected from decahydro-(iso)quinoline, tetrahydro-(iso)quinoline, piperazine, piperidine, indole, (iso)indole, benzyl, furan, or is selected from subformula (Ia) through subformula (If):

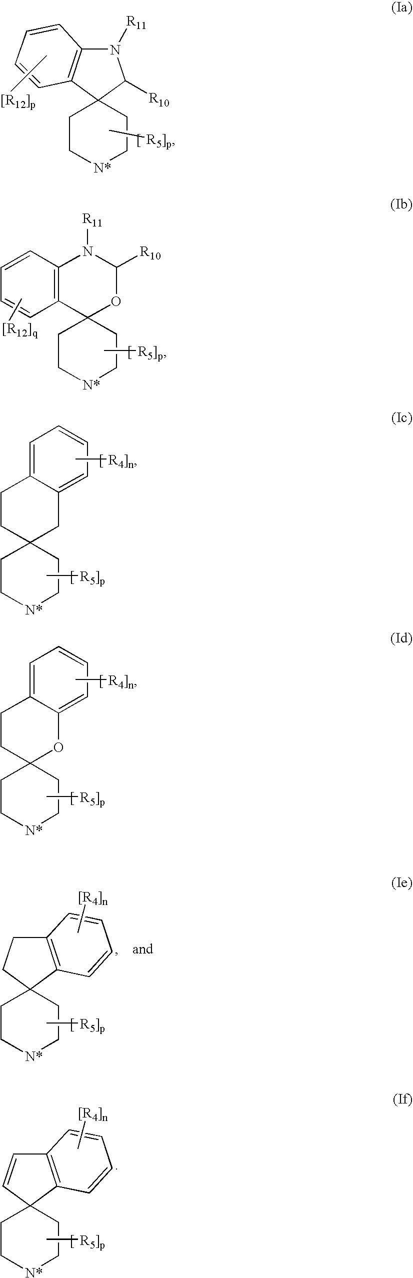

wherein N* designates the N to which is attached the peptide bond of formula I (i.e., is further substituted by —C(O)—CR₁R₂R₃), wherein R₁, R₂ and R₃ are as defined above.

In other embodiments, the invention provides compounds in which at least one of R₁, R₂, or R₃ is selected from hydrogen. In related embodiments, the invention provides compounds in which at least one of R₁, R₂, or R₃ is selected from the group of NH₆ or NH₂. In a preferred embodiment, the invention provides compounds in which R₁ is NH₂, and R₂ is H.

Unless specifically stated, reference to any of the R groups in any of the provided formulations does not infer chirality or stereospecificity.

In certain embodiments, a compound of the present invention is further characterized as modulator of a histone deacetylase (“HDAC”), including a mammalian HDAC, and especially including a human HDAC polypeptide. In a preferred embodiment, the aminoamine compound of the invention is a HDAC inhibitor. A preferred HDAC inhibitor is a non-hydroxamate, non-thio containing compound of the invention.

In embodiments related to these uses and methods, the disease includes a proliferative disease, which includes a benign or malignant tumor, a carcinoma of the brain, kidney, liver, adrenal gland, bladder, breast, stomach (for example gastric tumors), ovaries, esophagus, colon, rectum, prostate, pancreas, lung, vagina, thyroid, sarcoma, glioblastomas, multiple myeloma or gastrointestinal cancer, for example, colon carcinoma or colorectal adenoma, or a tumor of the neck and head, an epidermal hyperproliferation, for example, psoriasis, prostate hyperplasia, a neoplasia, including a neoplasia of epithelial character, including mammary carcinoma, or a leukemia.

In still another related embodiment, the disease to be treated by the uses and methods of the present invention is selected from triggering by persistent proliferative conditions such as angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and treating age spots and contact dermatitis.

In a related embodiment, the diseases to be treated by the uses and methods of the present invention include diseases and ailments associated with misregulated gene expression. The term “misregulated gene expression” includes altered levels of expression either by increased expression, decreased expression, and includes changes in temporal expression, or a combination thereof, compared to normal.

In embodiments related to these uses and methods, the disease includes a hyperproliferative disease, which includes leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds.

In other embodiments, the invention provides a kit including any of the compounds of the present invention. In a related embodiment, the kit further includes a pharmaceutically acceptable carrier or excipient of any of these compounds. In another related embodiment, the compounds of the invention, present in the kit, are in a unit dose. In still another related embodiment, the kit further includes instructions for use in administering to a subject.

As is evident to those skilled in the art, many of the compounds of the present invention contain asymmetric carbon atoms. It should be understood, therefore, that all individual stereoisomers of the provided formulas are contemplated as being included within the scope of this invention.

The compounds of the present invention are suitable as active agents in pharmaceutical compositions that are efficacious particularly for treating cellular proliferative ailments. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially an anti-tumor effect, e.g., inhibition of proliferation of malignant cancer cells, benign tumor cells or other proliferative cells, or of any other HDAC dependent disease.

As is evident to those skilled in the art, the many of the carboxyamine compounds of the present invention contain asymmetric carbon atoms. It should be understood, therefore, that the individual stereoisomers are contemplated as being included within the scope of this invention.

A HDAC dependent disease is a disease associated with a mutated HDAC polypeptide, with misregulation of a HDAC polypeptide, or is discovered to respond to inhibition of at least one HDAC polypeptide. HDAC dependent diseases include, e.g., those that depend on activity or misregulation of at least one of HDAC1 (Online Mendelian Inheritance in Man (“OMIM”) accno. 601241), HDAC2, HDAC3 (OMIM accno. 605166), HDAC4 (OMIM accno. 605314), HDAC5 (OMIM accno. 605315), HDAC6, HDAC7, HDAC8 (OMIM accno. 300269), HDAC9 (OMIM accno. 606543), HDAC10 (OMIM accno. 608544), HDAC11 (OMIM accno. 607226), and BRAF35/HDAC complex 80-KD subunit (OMIM accno. 608325), or an HDAC-associated pathway, or a disease dependent on any two or more of the HDACs just mentioned. OMIM is a database of gene-associated diseases maintained by Johns Hopkins University and publicly available through the National Center for Biotechnology Information at the U.S. National Institutes of Health.

In one embodiment, the diseases to be treated by compounds of the invention include, for example, a proliferative disease, preferably a benign or especially malignant tumor, more preferably carcinoma of the brain, kidney, liver, adrenal gland, bladder, breast, stomach (including gastric tumors), esophagus, ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head; an epidermal hyperproliferation, especially psoriasis, prostate hyperplasia, a neoplasia, including those of epithelial character, for example mammary carcinoma, or a leukemia.

In a further embodiment, the disease to be treated is a disease that is triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue.

The compounds of the present invention can also be used for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

In specific embodiments, the present invention provides the following compounds: 4-Biphenyl-3-yl-1,2,3,6-tetrahydro-pyridine; [2-(4-Benzofuran-2-yl-3,6-dihydro-2H-pyridin-1-yl)-2-oxo-ethyl]-carbamic acid tert-butyl ester; [2-(4-Biphenyl-3-yl-3,6-dihydro-2H-pyridin-1-yl)-2-oxo-ethyl]-carbamic acid tert-butyl ester; 2-Amino-1-(4-benzofuran-2-yl-3,6-dihydro-2H-pyridin-1-yl)-ethanone; 2-Amino-1-(4-biphenyl-3-yl-3,6-dihydro-2H-pyridin-1-yl)-ethanone; 2-Amino-1-(4-biphenyl-3-yl-piperidin-1-yl)-ethanone; 2-Amino-1-[4-(2-morpholin-4-ylmethyl-phenyl)-3,6-dihydro-2H-pyridin-1-yl]-ethanone; 2-Amino-1-(4-benzhydryl-piperazin-1-yl)-ethanone; N-(2-Acetyl-2,3-dihydro-1H-isoindol-5-yl)-benzamide; 2-Amino-1-[4-(5-chloro-2-methyl-phenyl)-piperazin-1-yl]-5-phenyl-pent-4-en-1-one; 2-Amino-1-(4-benzhydryl-piperazin-1-yl)-5-phenyl-pent-4-en-1-one; N-{2-[2-Amino-3-(2,4-dichloro-phenyl)-propionyl]-2,3-dihydro-1H-isoindol-5-yl}-5-fluoro-2-trifluoromethyl-benzamide; N-{2-[2-Amino-3-(2,4-dichloro-phenyl)-propionyl]-2,3-dihydro-1H-isoindol-5-yl}-5-chloro-2-trifluoromethyl-benzamide; 2-Amino-3-(4-chloro-phenyl)-1-[4-(3-chloro-phenyl)-piperidin-1-yl]-propan-1-one; 2-Amino-3-(4-chloro-phenyl)-1-[4-(3-chloro-phenyl)-3,6-dihydro-2H-pyridin-1-yl]-propan-1-one; 2-Amino-1-(4-biphenyl-3-yl-3,6-dihydro-2H-pyridin-1-yl)-3-(4-chloro-phenyl)-propan-1-one; 2-Amino-1-[4-(5-chloro-2-methyl-phenyl)-3,6-dihydro-2H-pyridin-1-yl]-3-(4-chloro-phenyl)-propan-1-one; {1-(4-(4-Chloro-benzyl)-2-[4-(5-chloro-2-methyl-phenyl)-3,6-dihydro-2H-pyridin-1-yl]-2-oxo-ethyl}-carbamic acid tert-butyl ester; [2-(4-Benzofuran-2-yl-3,6-dihydro-2H-pyridin-1-yl)-1-(4-chloro-benzyl)-2-oxo-ethyl]-carbamic acid tert-butyl ester; [2-(4-Biphenyl-3-yl-piperidin-1-yl)-1-(4-chloro-benzyl)-2-oxo-ethyl]-carbamic acid tert-butyl ester; 2-Amino-1-(4-biphenyl-3-yl-piperidin-1-yl)-3-(4-chloro-phenyl)-propan-1-one; 2-Amino-1-(4-benzofuran-2-yl-3,6-dihydro-2H-pyridin-1-yl)-3-(4-chloro-phenyl)-propan-1-one; 2-Amino-3-(4-benzyloxy-phenyl)-1-(4-biphenyl-3-yl-piperidin-1-yl)-propan-1-one; 2-Amino-3-(4-chloro-phenyl)-1-[4-(2-morpholin-4-ylmethyl-phenyl)-3,6-dihydro-2H-pyridin-1-yl]-propan-1-one; 2-Amino-1-(4-biphenyl-3-yl-piperidin-1-yl)-3-(3,4-dichloro-phenyl)-propan-1-one; 2-Amino-1-(4-benzhydryl-piperazin-1-yl)-3-(4-chloro-phenyl)-propan-1-one; 2-Amino-1-(4-biphenyl-3-yl-piperidin-1-yl)-3-pyridin-4-yl-propan-1-one; 2-Amino-1-(4-biphenyl-3-yl-piperidin-1-yl)-3-(4-hydroxy-phenyl)-propan-1-one; 1-(4-Biphenyl-3-yl-piperidin-1-yl)-3-(4-chloro-phenyl)-2-methylamino-propan-1-one; 1-(4-Biphenyl-3-yl-3,6-dihydro-2H-pyridin-1-yl)-3-(4-chloro-phenyl)-2-methylamino-propan-1-one; 2-Amino-1-(4-biphenyl-3-yl-piperidin-1-yl)-3-(2,4-dichloro-phenyl)-propan-1-one; 2-Amino-1-(4-biphenyl-3-yl-piperidin-1-yl)-3-p-tolyl-propan-1-one; 2-Amino-3-(4-chloro-phenyl)-1-[4-(4-fluoro-phenyl)-piperazin-1-yl]-propan-1-one; 2-Amino-1-(4-biphenyl-3-yl-piperidin-1-yl)-2-phenyl-ethanone; 2-Amino-1-(4-benzhydryl-piperazin-1-yl)-2-phenyl-ethanone; 2-Amino-3-(3,4-dichloro-phenyl)-1-[4-(4-fluoro-phenyl)-piperazin-1-yl]-propan-1-one; 2-Amino-1-(4-benzhydryl-piperazin-1-yl)-3-naphthalen-1-yl-propan-1-one; 2-Amino-1-(4-benzhydryl-piperazin-1-yl)-3-phenyl-propan-1-one; 4-[2-Amino-3-(4-benzhydryl-piperazin-1-yl)-3-oxo-propyl]-benzonitrile; 2-Amino-1-(4-biphenyl-3-yl-piperidin-1-yl)-3-phenyl-propan-1-one; 2-Amino-3-(4-chloro-phenyl)-1-(4-naphthalen-1-yl-3,6-dihydro-2H-pyridin-1-yl)-propan-1-one; 2-Amino-1-(4-benzhydryl-piperazin-1-yl)-3-biphenyl-4-yl-propan-1-one; 2-Amino-1-(4-benzofuran-2-yl-3,6-dihydro-2H-pyridin-1-yl)-3-(2,4-dichloro-phenyl)-propan-1-one; 2-Amino-3-(2,4-dichloro-phenyl)-1-[4-(3-fluoro-phenyl)-3,6-dihydro-2H-pyridin-1-yl]-propan-1-one; 2-Amino-1-(4-benzhydryl-piperazin-1-yl)-3-(2-chloro-phenyl)-propan-1-one; 2-[2-Amino-3-(4-benzhydryl-piperazin-1-yl)-3-oxo-propyl]-benzonitrile; 2-Amino-1-[4-(3-fluoro-benzyl)-piperazin-1-yl]-3-phenyl-propan-1-one; 2-Amino-1-[4-(4-fluoro-phenyl)-piperazin-1-yl]-3-p-tolyl-propan-1-one; 2-Amino-3-(4-benzyloxy-phenyl)-1-[4-(4-fluoro-phenyl)-piperazin-1-yl]-propan-1-one; 4-{2-Amino-3-[4-(4-fluoro-phenyl)-piperazin-1-yl]-3-oxo-propyl}-benzonitrile; 2-Amino-3-biphenyl-4-yl-1-[4-(4-fluoro-phenyl)-piperazin-1-yl]-propan-1-one; 2-Amino-1-[4-(4-fluoro-phenyl)-piperazin-1-yl]-3-phenyl-propan-1-one; 2-Amino-1-(4-benzhydryl-piperazin-1-yl)-3-(2,4-dichloro-phenyl)-propan-1-one; 2-Amino-3-(2,4-dichloro-phenyl)-1-[4-(3-fluoro-benzyl)-piperazin-1-yl]-propan-1-one; 2-Amino-3-(2-chloro-phenyl)-1-[4-(3-fluoro-benzyl)-piperazin-1-yl]-propan-1-one; 2-Amino-3-(4-chloro-phenyl)-1-[4-(3-fluoro-benzyl)-piperazin-1-yl]-propan-1-one; N-(3-{1-[2-Amino-3-(2,4-dichloro-phenyl)-propionyl]-piperidin-4-yl}-phenyl)-acetamide; N-(3-{1-[2-Amino-3-(2,4-dichloro-phenyl)-propionyl]-piperidin-4-yl}-phenyl)-benzamide; 2-Amino-1-(4-benzhydryl-piperazin-1-yl)-3-thiophen-3-yl-propan-1-one; 2-Amino-1-(4-benzhydryl-piperazin-1-yl)-3-thiophen-2-yl-propan-1-one; 2-Amino-1-[4-(5-chloro-2-methyl-phenyl)-piperazin-1-yl]-3-thiophen-2-yl-propan-1-one; 2-Amino-1-[4-(5-chloro-2-methyl-phenyl)-piperazin-1-yl]-3-phenyl-propan-1-one; 2-Amino-1-[4-(5-chloro-2-methyl-phenyl)-piperazin-1-yl]-3-(4-chloro-phenyl)-propan-1-one; 2-Amino-1-[4-(5-chloro-2-methyl-phenyl)-piperazin-1-yl]-3-(2,4-dichloro-phenyl)-propan-1-one; 2-Amino-3-biphenyl-4-yl-1-[4-(5-chloro-2-methyl-phenyl)-piperazin-1-yl]-propan-1-one; 2-Amino-1-[4-(5-chloro-2-methyl-phenyl)-piperazin-1-yl]-3-(4-trifluoromethyl-phenyl)-propan-1-one; N-(3-{1-[2-Amino-3-(4-chloro-phenyl)-propionyl]-piperidin-4-yl}-phenyl)-benzamide; 2-Amino-1-(4-biphenyl-3-yl-piperidin-1-yl)-3-thiophen-2-yl-propan-1-one; 2-Amino-1-(4-biphenyl-3-yl-piperidin-1-yl)-3-thiophen-3-yl-propan-1-one; 2-Amino-1-[4-(5-chloro-2-methyl-phenyl)-piperazin-1-yl]-3-thiophen-3-yl-propan-1-one; [2-[4-(5-Chloro-2-methyl-phenyl)-piperazin-1-yl]-1-(2,4-dichloro-benzyl)-2-oxo-ethyl]-carbamic acid tert-butyl ester; 2-Amino-1-(4-benzofuran-2-yl-piperidin-1-yl)-3-(2,4-dichloro-phenyl)-propan-1-one; Thioacetic acid -[2-(4-benzhydryl-piperazin-1-yl)-1-benzyl-2-oxo-ethyl]ester; 2-Amino-3-(5-bromo-thiophen-2-yl)-1-(4-naphthalen-1-yl-piperidin-1-yl)-propan-1-one; 2-Amino-3-(5-bromo-thiophen-2-yl)-1-[4-(5-chloro-2-methyl-phenyl)-piperazin-1-yl]-propan-1-one; 2-{2-Amino-3-[4-(5-chloro-2-methyl-phenyl)-piperazin-1-yl]-3-oxo-propyl}-benzonitrile; 2-Amino-3-(2-chloro-phenyl)-1-(4-naphthalen-1-yl-piperidin-1-yl)-propan-1-one; 2-Amino-1-(4-benzoyl-piperazin-1-yl)-3-(2-chloro-phenyl)-propan-1-one; 2-Amino-3-(2,4-dichloro-phenyl)-1-(4-naphthalen-1-yl-piperidin-1-yl)-propan-1-one; 2-Amino-3-(4-chloro-phenyl)-1-(4-naphthalen-1-yl-piperidin-1-yl)-propan-1-one; 2-Amino-1-[4-(5-chloro-2-methyl-phenyl)-piperazin-1-yl]-3-(2-chloro-phenyl)-propan-1-one; 2-Amino-3-(2,4-dichloro-phenyl)-1-[4-(4-fluoro-phenyl)-piperazin-1-yl]-propan-1-one; 2-Amino-3-(2-chloro-phenyl)-1-[4-(4-fluoro-phenyl)-piperazin-1-yl]-propan-1-one; 2-Amino-3-(5-bromo-thiophen-2-yl)-1-[4-(4-fluoro-phenyl)-piperazin-1-yl]-propan-1-one; 2-Amino-1-(4-benzhydryl-piperazin-1-yl)-3-(5-bromo-thiophen-2-yl)-propan-1-one; 2-Amino-1-(4-benzoyl-piperazin-1-yl)-3-(2,4-dichloro-phenyl)-propan-1-one; 2-Amino-1-[4-(5-chloro-2-methyl-phenyl)-piperazin-1-yl]-3-furan-2-yl-propan-1-one; 2-Amino-1-(4-benzhydryl-piperazin-1-yl)-3-thiazol-5-yl-propan-1-one; 2-Amino-1-(4-biphenyl-3-yl-piperidin-1-yl)-3-furan-2-yl-propan-1-one; 2-Amino-1-(4-biphenyl-3-yl-piperidin-1-yl)-5-phenyl-pent-4-en-1-one; 2-Amino-1-(4-biphenyl-3-yl-piperidin-1-yl)-3-(5-bromo-thiophen-2-yl)-propan-1-one; 2-Amino-1-(4-benzhydryl-piperazin-1-yl)-3-(3-chloro-phenyl)-propan-1-one; 2-Amino-1-(4-benzhydryl-piperazin-1-yl)-3-(3-methyl-3H-imidazol-4-yl)-propan-1-one; 2-Amino-1-(4-benzhydryl-piperazin-1-yl)-3-(4-fluoro-phenyl)-propan-1-one; 2-Amino-1-(4-benzhydryl-piperazin-1-yl)-3-(2-fluoro-phenyl)-propan-1-one; 2-Amino-1-(4-benzhydryl-piperazin-1-yl)-3-o-tolyl-propan-1-one; 2-Amino-1-[4-(5-chloro-2-methyl-phenyl)-piperazin-1-yl]-3-(3-trifluoromethyl-phenyl)-propan-1-one; 2-Amino-1-[4-(5-chloro-2-methyl-phenyl)-piperazin-1-yl]-3-(4-fluoro-phenyl)-propan-1-one; 2-Amino-1-[4-(5-chloro-2-methyl-phenyl)-piperazin-1-yl]-3-(3-chloro-phenyl)-propan-1-one; 2-Amino-1-[4-(5-chloro-2-methyl-phenyl)-piperazin-1-yl]-3-o-tolyl-propan-1-one; 2-Amino-1-[4-(5-chloro-2-methyl-phenyl)-piperazin-1-yl]-3-m-tolyl-propan-1-one; 2-Amino-1-[4-(5-chloro-2-methyl-phenyl)-piperazin-1-yl]-3-thiazol-4-yl-propan-1-one; 2-Amino-1-[4-(5-chloro-2-methyl-phenyl)-piperazin-1-yl]-3-(2-trifluoromethyl-phenyl)-propan-1-one; 2-Amino-1-[4-(5-chloro-2-methyl-phenyl)-piperazin-1-yl]-3-(3-methyl-3H-imidazol-4-yl)-propan-1-one; 2-Amino-1-(4-benzhydryl-piperazin-1-yl)-3-(2-trifluoromethyl-phenyl)-propan-1-one; 2-Amino-1-(4-benzhydryl-piperazin-1-yl)-3-m-tolyl-propan-1-one; 2-Amino-1-(4-benzhydryl-piperazin-1-yl)-3-(3-trifluoromethyl-phenyl)-propan-1-one; 2-Amino-1-(4-benzoyl-piperazin-1-yl)-3-thiazol-4-yl-propan-1-one; 2-Amino-1-(4-benzhydryl-piperazin-1-yl)-3-(1-methyl-1H-imidazol-4-yl)-propan-1-one; 2-Amino-1-(4-benzhydryl-piperazin-1-yl)-3-(1H-imidazol-4-yl)-propan-1-one; 2-Amino-1-(4-benzoyl-piperazin-1-yl)-3-(4-fluoro-phenyl)-propan-1-one; 2-Amino-1-(4-benzoyl-piperazin-1-yl)-3-(2-fluoro-phenyl)-propan-1-one; 2-Amino-1-(4-benzoyl-piperazin-1-yl)-3-(3-trifluoromethyl-phenyl)-propan-1-one; 2-Amino-1-(4-benzoyl-piperazin-1-yl)-3-(3-chloro-phenyl)-propan-1-one; 2-Amino-1-(4-benzoyl-piperazin-1-yl)-3-(2-trifluoromethyl-phenyl)-propan-1-one; 2-Amino-1-(4-benzoyl-piperazin-1-yl)-3-m-tolyl-propan-1-one; 2-Amino-1-(4-benzoyl-piperazin-1-yl)-3-o-tolyl-propan-1-one; 2-Amino-1-(4-benzoyl-piperazin-1-yl)-3-(3-methyl-3H-imidazol-4-yl)-propan-1-one; 2-Amino-1-(4-benzhydryl-piperazin-1-yl)-3-[5-(4-chloro-phenyl)-thiophen-2-yl]-propan-1-one; 2-Amino-1-(4-benzhydryl-piperazin-1-yl)-3-[5-(3-chloro-phenyl)-thiophen-2-yl]-propan-1-one; 2-Amino-1-(4-benzhydryl-piperazin-1-yl)-3-[5-(2-chloro-phenyl)-thiophen-2-yl]-propan-1-one; 2-Amino-1-(4-benzoyl-piperazin-1-yl)-3-(1H-imidazol-4-yl)-propan-1-one; 2-Amino-1-(4-benzoyl-piperazin-1-yl)-3-thiophen-3-yl-propan-1-one; 2-Amino-1-(4-benzoyl-piperazin-1-yl)-3-thiophen-2-yl-propan-1-one; 2-Amino-1-(4-benzoyl-piperazin-1-yl)-3-(5-bromo-thiophen-2-yl)-propan-1-one; 2-Amino-1-(4-benzoyl-piperazin-1-yl)-3-furan-2-yl-propan-1-one; 2-Amino-1-(4-benzhydryl-piperazin-1-yl)-3-[5-(2,4-dichloro-phenyl)-thiophen-2-yl]-propan-1-one; 2-Amino-1-[4-(5-chloro-2-methoxy-phenyl)-piperazin-1-yl]-3-(2,4-dichloro-phenyl)-propan-1-one; 2-Amino-1-[4-(5-chloro-2-methoxy-phenyl)-piperazin-1-yl]-3-(2-chloro-phenyl)-propan-1-one; 2-Amino-1-[4-(3-chloro-phenyl)-piperazin-1-yl]-3-(2,4-dichloro-phenyl)-propan-1-one; 2-Amino-3-(2-chloro-phenyl)-1-[4-(3-chloro-phenyl)-piperazin-1-yl]-propan-1-one; 2-Amino-3-(2,4-dichloro-phenyl)-1-{4-[3-(4-methyl-piperazin-1-ylmethyl)-phenyl]-piperidin-1-yl}-propan-1-one; 1-[4-(5-Chloro-2-methyl-phenyl)-piperazin-1-yl]-3-(2,4-dichloro-phenyl)-2-mercapto-propan-1-one; 2-Amino-1-[4-(3-chloro-phenyl)-piperazin-1-yl]-3-m-tolyl-propan-1-one; 2-Amino-3-(3-chloro-phenyl)-1-[4-(3-chloro-phenyl)-piperazin-1-yl]-propan-1-one; 2-Amino-1-[4-(5-chloro-2-methoxy-phenyl)-piperazin-1-yl]-3-(3-chloro-phenyl)-propan-1-one; 2-Amino-1-[4-(3-fluoro-benzyl)-piperazin-1-yl]-3-(3-trifluoromethyl-phenyl)-propan-1-one; 2-Amino-1-[4-(3-fluoro-benzyl)-piperazin-1-yl]-3-(2-trifluoromethyl-phenyl)-propan-1-one; 2-Amino-3-(3-chloro-phenyl)-1-[4-(3-fluoro-benzyl)-piperazin-1-yl]-propan-1-one; 2-Amino-3-(2,4-dichloro-phenyl)-1-piperazin-1-yl-propan-1-one; 2-Amino-3-(2,4-dichloro-phenyl)-1-morpholin-4-yl-propan-1-one; 2-Amino-3-(2,4-dichloro-phenyl)-1-(4-methyl-piperazin-1-yl)-propan-1-one; 2-Amino-3-(2,4-dichloro-phenyl)-1-[4-(5-fluoro-2-trifluoromethyl-benzoyl)-piperazin-1-yl]-propan-1-one; 2-Amino-3-(2-chloro-phenyl)-1-[4-(5-fluoro-2-trifluoromethyl-benzoyl)-piperazin-1-yl]-propan-1-one; 2-Amino-1-[4-(5-fluoro-2-trifluoromethyl-benzoyl)-piperazin-1-yl]-3-(2-trifluoromethyl-phenyl)-propan-1-one; 2-Amino-1-[4-(5-fluoro-2-trifluoromethyl-benzoyl)-piperazin-1-yl]-3-(2-trifluoromethyl-phenyl)-propan-1-one; 2-Amino-1-[4-(5-fluoro-2-trifluoromethyl-benzoyl)-piperazin-1-yl]-3-thiophen-2-yl-propan-1-one; 2-Amino-3-(5-bromo-thiophen-2-yl)-1-[4-(5-fluoro-2-trifluoromethyl-benzoyl)-piperazin-1-yl]-propan-1-one; 1-(4-Benzhydryl-piperazin-1-yl)-3-(2,4-dichloro-phenyl)-2-mercapto-propan-1-one; 2-Amino-3-(2,4-dichloro-phenyl)-1-(4-indan-2-yl-piperazin-1-yl)-propan-1-one; 2-Amino-3-(2,4-dichloro-phenyl)-1-(4-pyridin-2-ylmethyl-piperazin-1-yl)-propan-1-one; 2-Amino-1-(4-indan-2-yl-piperazin-1-yl)-3-thiophen-2-yl-propan-1-one; 2-Amino-3-(2,4-dichloro-phenyl)-1-[4-(3-thiophen-2-yl-benzyl)-piperazin-1-yl]-propan-1-one; 2-Amino-1-[4-(3′-chloro-biphenyl-3-ylmethyl)-piperazin-1-yl]-3-(2,4-dichloro-phenyl)-propan-1-one; 2-Amino-3-(2,4-dichloro-phenyl)-1-(4-pyridin-3-ylmethyl-piperazin-1-yl)-propan-1-one; 2-Amino-3-(2,4-dichloro-phenyl)-1-(4-pyridin-4-ylmethyl-piperazin-1-yl)-propan-1-one; 2-Amino-3-(2,4-dichloro-phenyl)-1-[4-(2,5-difluoro-benzyl)-piperazin-1-yl]-propan-1-one; 2-Amino-1-{4-[3-(3,5-dichloro-phenoxy)-benzyl]-piperazin-1-yl}-3-(2,4-dichloro-phenyl)-propan-1-one; 2-Amino-3-(2,4-dichloro-phenyl)-1-[4-(3-methyl-benzyl)-piperazin-1-yl]-propan-1-one; 2-Amino-3-(2,4-dichloro-phenyl)-1-[4-(4-fluoro-benzyl)-piperazin-1-yl]-propan-1-one; 2-Amino-3-(2,4-dichloro-phenyl)-1-[4-(pyridine-2-carbonyl)-piperazin-1-yl]-propan-1-one; 2-Amino-3-(2-chloro-phenyl)-1-[4-(pyridine-2-carbonyl)-piperazin-1-yl]-propan-1-one; 2-Amino-3-(3-chloro-phenyl)-1-[4-(pyridine-2-carbonyl)-piperazin-1-yl]-propan-1-one; 2-Amino-3-(5-bromo-thiophen-2-yl)-1-[4-(pyridine-2-carbonyl)-piperazin-1-yl]-propan-1-one; 2-Amino-3-(2,4-dichloro-phenyl)-1-[4-(4-methoxy-benzyl)-piperazin-1-yl]-propan-1-one; 1-(4-Benzhydryl-piperazin-1-yl)-3-(3-chloro-phenyl)-2-mercapto-propan-1-one; 1-[4-(5-Chloro-2-methyl-phenyl)-piperazin-1-yl]-3-(3-chloro-phenyl)-2-mercapto-propan-1-one; 3-(3-Chloro-phenyl)-1-[4-(5-fluoro-2-trifluoromethyl-benzoyl)-piperazin-1-yl]-2-mercapto-propan-1-one; 2-Amino-3-(2,4-dichloro-phenyl)-1-[4-(4-methyl-benzyl)-piperazin-1-yl]-propan-1-one; 2-Amino-1-(4-cyclohexanecarbonyl-piperazin-1-yl)-3-(2,4-dichloro-phenyl)-propan-1-one; 2-Amino-1-{4-[bis-(4-fluoro-phenyl)-methyl]-piperazin-1-yl}-3-(2,4-dichloro-phenyl)-propan-1-one; 2-Amino-3-(2,4-dichloro-phenyl)-1-[4-(4-pyrimidin-5-yl-benzyl)-piperazin-1-yl]-propan-1-one; 2-Amino-3-(2,4-dichloro-phenyl)-1-[4-(4-pyridin-4-yl-benzyl)-piperazin-1-yl]-propan-1-one; 2-Amino-3-(2,4-dichloro-phenyl)-1-[4-(3-fluoro-pyridin-4-ylmethyl)-piperazin-1-yl]-propan-1-one; 2-Amino-1-[4-(3,4-dichloro-benzyl)-piperazin-1-yl]-3-(2,4-dichloro-phenyl)-propan-1-one; 2-Amino-1-[4-(pyridine-2-carbonyl)-piperazin-1-yl]-3-(2-trifluoromethyl-phenyl)-propan-1-one; 2-Amino-3-(2-fluoro-phenyl)-1-[4-(pyridine-2-carbonyl)-piperazin-1-yl]-propan-1-one; 2-Amino-3-(5-bromo-thiophen-2-yl)-1-(4-indan-2-yl-piperazin-1-yl)-propan-1-one; 2-Amino-1-[4-(6-chloro-pyridin-2-yl)-piperazin-1-yl]-3-(2,4-dichloro-phenyl)-propan-1-one; 2-Amino-1-[4-(5-chloro-2-trifluoromethyl-benzoyl)-piperazin-1-yl]-3-(2,4-dichloro-phenyl)-propan-1-one; 2-Amino-3-(2-chloro-phenyl)-1-[4-(5-chloro-2-trifluoromethyl-benzoyl)-piperazin-1-yl]-propan-1-one; 2-Amino-3-(3-chloro-phenyl)-1-[4-(5-chloro-2-trifluoromethyl-benzoyl)-piperazin-1-yl]-propan-1-one; 2-Amino-1-[4-(5-chloro-2-trifluoromethyl-benzoyl)-piperazin-1-yl]-3-(2-trifluoromethyl-phenyl)-propan-1-one; 2-Amino-1-[4-(5-chloro-2-trifluoromethyl-benzoyl)-piperazin-1-yl]-3-thiophen-2-yl-propan-1-one; 2-Amino-3-(5-bromo-thiophen-2-yl)-1-[4-(5-chloro-2-trifluoromethyl-benzoyl)-piperazin-1-yl]-propan-1-one; 2-Amino-3-(5-bromo-thiophen-2-yl)-1-[4-(5-chloro-2-methoxy-phenyl)-piperazin-1-yl]-propan-1-one; 2-Amino-3-(2,4-dichloro-phenyl)-1-[4-(2,5-difluoro-benzoyl)-piperazin-1-yl]-propan-1-one; 2-Amino-3-(2-chloro-phenyl)-1-[4-(2,5-difluoro-benzoyl)-piperazin-1-yl]-propan-1-one 2-Amino-3-(3-chloro-phenyl)-1-[4-(2,5-difluoro-benzoyl)-piperazin-1-yl]-propan-1-one; 2-Amino-1-[4-(2,5-difluoro-benzoyl)-piperazin-1-yl]-3-(2-trifluoromethyl-phenyl)-propan-1-one; 2-Amino-1-[4-(2,5-difluoro-benzoyl)-piperazin-1-yl]-3-thiophen-2-yl-propan-1-one; 2-Amino-3-(5-bromo-thiophen-2-yl)-1-[4-(2,5-difluoro-benzoyl)-piperazin-1-yl]-propan-1-one; 2-Amino-1-[4-(2,5-difluoro-benzoyl)-piperazin-1-yl]-3-(3-trifluoromethyl-phenyl)-propan-1-one; 2-Amino-1-(4-benzhydryl-piperazin-1-yl)-3-[5-(2,6-dichloro-phenyl)-thiophen-2-yl]-propan-1-one; 2-Amino-1-(4-benzhydryl-piperazin-1-yl)-3-[2,2′]bithiophenyl-5-yl-propan-1-one; 2-Amino-1-[4-(3-bromo-phenyl)-piperazin-1-yl]-3-(2,4-dichloro-phenyl)-propan-1-one; 2-Amino-1-[4-(5-chloro-2-methoxy-phenyl)-piperazin-1-yl]-3-[5-(2,4-dichloro-phenyl)-thiophen-2-yl]-propan-1-one; 2-Amino-1-(4-benzhydryl-piperazin-1-yl)-3-[5-(2,4-difluoro-phenyl)-thiophen-2-yl]-propan-1-one; 2-Amino-3-(5-bromo-thiophen-2-yl)-1-(4-methyl-piperazin-1-yl)-propan-1-one; 2-Amino-3-(2,4-dichloro-phenyl)-1-[4-(3,4-dichloro-phenyl)-piperazin-1-yl]-propan-1-one; 2-Amino-1-[4-(3,4-dichloro-phenyl)-piperazin-1-yl]-3-thiophen-2-yl-propan-1-one; 2-Amino-1-(4-pyridin-2-ylmethyl-piperazin-1-yl)-3-thiophen-2-yl-propan-1-one; 2-Amino-3-(5-bromo-thiophen-2-yl)-1-[4-(3,4-dichloro-phenyl)-piperazin-1-yl]-propan-1-one; 2-Amino-1-[4-(3′-chloro-biphenyl-3-yl)-piperazin-1-yl]-3-(2,4-dichloro-phenyl)-propan-1-one; 2-Amino-1-[4-(2,4-dichloro-5-fluoro-pyridine-3-carbonyl)-piperazin-1-yl]-3-(2,4-dichloro-phenyl)-propan-1-one; 2-Amino-3-(3-chloro-phenyl)-1-[4-(2,4-dichloro-5-fluoro-pyridine-3-carbonyl)-piperazin-1-yl]-propan-1-one; 2-Amino-1-[4-(2,4-dichloro-5-fluoro-pyridine-3-carbonyl)-piperazin-1-yl]-3-(2-trifluoromethyl-phenyl)-propan-1-one; 2-Amino-1-[4-(2,4-dichloro-5-fluoro-pyridine-3-carbonyl)-piperazin-1-yl]-3-thiophen-2-yl-propan-1-one; 2-Amino-3-(5-bromo-thiophen-2-yl)-1-[4-(2,4-dichloro-5-fluoro-pyridine-3-carbonyl)-piperazin-1-yl]-propan-1-one; 2-Amino-1-[4-(2,4-dichloro-5-fluoro-pyridine-3-carbonyl)-piperazin-1-yl]-3-(3-trifluoromethyl-phenyl)-propan-1-one; 2-Amino-3-(5-bromo-thiophen-2-yl)-1-(4-pyridin-2-ylmethyl-piperazin-1-yl)-propan-1-one; 2-Amino-1-(4-methyl-piperazin-1-yl)-3-thiophen-2-yl-propan-1-one; 2-Amino-1-(4-biphenyl-3-yl-piperazin-1-yl)-3-(2,4-dichloro-phenyl)-propan-1-one; 2-Amino-1-(4-benzhydryl-piperazin-1-yl)-3-[5-(2-trifluoromethoxy-phenyl)-thiophen-2-yl]-propan-1-one; 2-Amino-1-(4-benzhydryl-piperazin-1-yl)-3-[5-(5-fluoro-2-methyl-phenyl)-thiophen-2-yl]-propan-1-one; 2-Amino-1-(4-benzhydryl-piperazin-1-yl)-3-[5-(2-chloro-4-trifluoromethyl-phenyl)-thiophen-2-yl]-propan-1-one; 2-Amino-1-[4-(5-chloro-2-methyl-phenyl)-piperazin-1-yl]-3-[5-(2,4-dichloro-phenyl)-thiophen-2-yl]-propan-1-one; 2-Amino-3-(2-chloro-phenyl)-1-[4-(2,4-dichloro-5-fluoro-pyridine-3-carbonyl)-piperazin-1-yl]-propan-1-one; 2-Amino-1-(4-benzhydryl-piperazin-1-yl)-3-[5-(4-fluoro-2-methyl-phenyl)-thiophen-2-yl]-propan-1-one; 2-Amino-3-[5-(2,4-dichloro-phenyl)-thiophen-2-yl]-1-(4-indan-2-yl-piperazin-1-yl)-propan-1-one; 2-Amino-3-(2,4-dichloro-phenyl)-1-[4-(3-pyridin-3-yl-phenyl)-piperazin-1-yl]-propan-1-one; 2-Amino-1-(4-benzhydryl-piperazin-1-yl)-3-[5-(2,4-bis-trifluoromethyl-phenyl)-thiophen-2-yl]-propan-1-one; 2-Amino-3-(5-bromo-thiophen-2-yl)-1-(4-pyridin-4-ylmethyl-piperazin-1-yl)-propan-1-one; 2-Amino-3-[5-(2,4-dichloro-phenyl)-thiophen-2-yl]-1-(4-methyl-piperazin-1-yl)-propan-1-one; 2-Amino-3-(5-bromo-thiophen-2-yl)-1-[4-(4-methoxy-benzyl)-piperazin-1-yl]-propan-1-one; 2-Amino-3-(2-chloro-phenyl)-1-[4-(6-chloro-pyridin-2-yl)-piperazin-1-yl]-propan-1-one; 2-Amino-3-(3-chloro-phenyl)-1-[4-(6-chloro-pyridin-2-yl)-piperazin-1-yl]-propan-1-one; 2-Amino-3-(5-bromo-thiophen-2-yl)-1-[4-(6-chloro-pyridin-2-yl)-piperazin-1-yl]-propan-1-one; 2-Amino-1-[4-(6-chloro-pyridin-2-yl)-piperazin-1-yl]-3-[5-(2,4-dichloro-phenyl)-thiophen-2-yl]-propan-1-one; 2-Amino-3-[5-(2,4-dichloro-phenyl)-thiophen-2-yl]-1-(4-pyridin-2-ylmethyl-piperazin-1-yl)-propan-1-one; 2-Amino-1-(4-benzhydryl-piperazin-1-yl)-3-pyridin-3-yl-propan-1-one; 2-Amino-1-(4-benzhydryl-piperazin-1-yl)-3-(5-pyridin-3-yl-thiophen-2-yl)-propan-1-one; 2-Amino-1-(4-benzhydryl-piperazin-1-yl)-3-(5-phenyl-thiophen-2-yl)-propan-1-one; 2-Amino-3-[5-(2,4-dichloro-phenyl)-thiophen-2-yl]-1-[4-(4-methoxy-benzyl)-piperazin-1-yl]-propan-1-one; 2-Amino-3-[5-(2,4-dichloro-phenyl)-thiophen-2-yl]-1-(4-pyridin-4-ylmethyl-piperazin-1-yl)-propan-1-one; 2-Amino-1-[4-(2,5-difluoro-benzoyl)-piperazin-1-yl]-3-pyridin-3-yl-propan-1-one; 2-Amino-3-pyridin-3-yl-1-(4-pyridin-4-ylmethyl-piperazin-1-yl)-propan-1-one; 2-Amino-1-[4-(pyridine-3-carbonyl)-piperazin-1-yl]-3-(3-trifluoromethyl-phenyl)-propan-1-one; 2-Amino-1-[4-(5-chloro-2-trifluoromethyl-benzoyl)-piperazin-1-yl]-3-(3-trifluoromethyl-phenyl)-propan-1-one; 2-Amino-1-[4-(5-fluoro-2-trifluoromethyl-benzoyl)-piperazin-1-yl]-3-(3-trifluoromethyl-phenyl)-propan-1-one; 2-Amino-1-(4-methyl-piperazin-1-yl)-3-[5-(2-methyl-4-propoxy-phenyl)-thiophen-2-yl]-propan-1-one; 2-Amino-1-(4-pyridin-4-ylmethyl-piperazin-1-yl)-3-(3-trifluoromethyl-phenyl)-propan-1-one; 2-Amino-1-(4-benzoyl-piperazin-1-yl)-3-[5-(2,4-dichloro-phenyl)-thiophen-2-yl]-propan-1-one; 2-Amino-3-(3-chloro-phenyl)-1-(4-pyridin-4-ylmethyl-piperazin-1-yl)-propan-1-one; 2-Amino-3-[5-(4-chloro-2-trifluoromethyl-phenyl)-thiophen-2-yl]-1-(4-methyl-piperazin-1-yl)-propan-1-one; 2-Amino-1-(4-pyridin-4-ylmethyl-piperazin-1-yl)-3-(2-trifluoromethyl-phenyl)-propan-1-one; 2-Amino-1-(4-pyridin-4-ylmethyl-piperazin-1-yl)-3-m-tolyl-propan-1-one; 2-Amino-1-(4-benzhydryl-piperazin-1-yl)-3-(4-bromo-phenyl)-propan-1-one; 2-Amino-1-(4-benzhydryl-piperazin-1-yl)-3-(2′,4′-dichloro-biphenyl-4-yl)-propan-1-one; 2-Amino-3-(4-amino-phenyl)-1-[4-(6-chloro-pyridin-2-yl)-piperazin-1-yl]-propan-1-one; (4-{2-Amino-3-[4-(6-chloro-pyridin-2-yl)-piperazin-1-yl]-3-oxo-propyl}-phenylamino)-acetic acid ethyl ester; N-{2-[2-Amino-3-(2,4-dichloro-phenyl)-propionyl]-2,3-dihydro-1H-isoindol-5-yl}-benzamide; N-{2-[2-Amino-3-(3-chloro-phenyl)-propionyl]-2,3-dihydro-1H-isoindol-5-yl}-benzamide; N-{2-[2-Amino-3-(5-bromo-thiophen-2-yl)-propionyl]-2,3-dihydro-1H-isoindol-5-yl}-benzamide; N-{2-[2-Amino-3-(2,4-dichloro-phenyl)-propionyl]-2,3-dihydro-1H-isoindol-5-yl}-2,5-difluoro-benzamide; 2-Amino-1-(4-biphenyl-3-yl-piperidin-1-yl)-3-(3,4-dichloro-phenyl)-propan-1-one; Thioacetic acid {1-benzyl-2-[4-(5-chloro-2-methyl-phenyl)-piperazin-1-yl]-2-oxo-ethyl}ester; 1-(4-Benzhydryl-piperazin-1-yl)-2-mercapto-3-phenyl-propan-1-one; 2-Amino-3-benzothiazol-2-yl-1-[4-(2,5-difluoro-benzoyl)-piperazin-1-yl]-propan-1-one; 2-Amino-3-benzo[b]thiophen-3-yl-1-[4-(2,5-difluoro-benzoyl)-piperazin-1-yl]-propan-1-one; 2-Amino-1-[4-(2,5-difluoro-benzoyl)-piperazin-1-yl]-2-thiophen-3-yl-ethanone; 2-Amino-3-benzothiazol-2-yl-1-[4-(pyridine-3-carbonyl)-piperazin-1-yl]-propan-1-one; 2-Amino-3-benzo[b]thiophen-3-yl-1-[4-(pyridine-3-carbonyl)-piperazin-1-yl]-propan-1-one; 2-Amino-1-[4-(pyridine-3-carbonyl)-piperazin-1-yl]-2-thiophen-3-yl-ethanone; 2-Amino-1-[4-(5-chloro-2-methyl-phenyl)-piperazin-1-yl]-3-(2,4-dichloro-phenyl)-propan-1-one; 2-Amino-1-[4-(5-chloro-2-methyl-phenyl)-piperazin-1-yl]-3-(3-chloro-phenyl)-propan-1-one; 2-Amino-1-(4-benzhydryl-piperazin-1-yl)-3-(2,4-dichloro-phenyl)-propan-1-one; 2-Amino-1-(4-benzhydryl-piperazin-1-yl)-3-(3-chloro-phenyl)-propan-1-one; and 2-Amino-1-(4-benzoyl-piperazin-1-yl)-3-(3-chloro-phenyl)-propan-1-one; 2-Amino-1-(1,3-dihydro-isoindol-2-yl)-3-(4-propyl-phenyl)-propan-1-one; (E)-(R)-2-Amino-1-(1,3-dihydro-isoindol-2-yl)-5-phenyl-pent-4-en-1-one; (R)-2-Amino-3-biphenyl-4-yl-1-(1,3-dihydro-isoindol-2-yl)-propan-1-one; 3-{4-[(R)-2-Amino-3-(1,3-dihydro-isoindol-2-yl)-3-oxo-propyl]-phenyl}-propionic acid methyl ester; (R)-2-Amino-1-(1,3-dihydro-isoindol-2-yl)-3-(4-hydroxy-phenyl)-propan-1-one; (R)-2-Amino-3-cyclohexyl-1-(1,3-dihydro-isoindol-2-yl)-propan-1-one; (R)-2-Amino-1-(1,3-dihydro-isoindol-2-yl)-4-methylsulfanyl-butan-1-one; (R)-2-Amino-1-(1,3-dihydro-isoindol-2-yl)-4-methylsulfanyl-butan-1-one; (R)-2-Amino-1-(1,3-dihydro-isoindol-2-yl)-4-methylsulfanyl-butan-1-one; (S)-2-Amino-3-(4-chloro-phenyl)-1-(1,3-dihydro-isoindol-2-yl)-propan-1-one; (R)-2-Amino-1-(1,3-dihydro-isoindol-2-yl)-3-(4-nitro-phenyl)-propan-1-one; (R)-2-Amino-3-(3,5-difluoro-phenyl)-1-(1,3-dihydro-isoindol-2-yl)-propan-1-one; (R)-2-Amino-3-(4-benzyloxy-phenyl)-1-(1,3-dihydro-isoindol-2-yl)-propan-1-one; (R)-4-Amino-5-(1,3-dihydro-isoindol-2-yl)-5-oxo-pentanoic acid; 2-Amino-1-(1,3-dihydro-isoindol-2-yl)-3-o-tolyl-propan-1-one; (R)-4-Amino-5-(1,3-dihydro-isoindol-2-yl)-5-oxo-pentanoic acid benzyl ester; (E)-3-{4-[(R)-2-Amino-3-(1,3-dihydro-isoindol-2-yl)-3-oxo-propyl]-phenyl}-acrylic acid methyl ester; {4-[(R)-2-Amino-3-(1,3-dihydro-isoindol-2-yl)-3-oxo-propyl]-phenoxy}-acetic acid methyl ester; 2-Amino-2-biphenyl-4-yl-1-(1,3-dihydro-isoindol-2-yl)-ethanone; (R)-3-Amino-4-(1,3-dihydro-isoindol-2-yl)-4-oxo-butyric acid benzyl ester; (R)-2-Amino-1-(1,3-dihydro-isoindol-2-yl)-4-(4-methyl-benzylsulfanyl)-butan-1-one; (R)-2-Amino-1-(1,3-dihydro-isoindol-2-yl)-2-(4-fluoro-phenyl)-ethanone; 2-Amino-1-(1,3-dihydro-isoindol-2-yl)-3-(2,4-dimethyl-phenyl)-propan-1-one; (R)-4-Amino-5-(1,3-dihydro-isoindol-2-yl)-5-oxo-pentanoic acid cyclohexyl ester; (R)-2-Amino-1-(1,3-dihydro-isoindol-2-yl)-3-(2-fluoro-phenyl)-propan-1-one; (R)-2-Amino-1-(1,3-dihydro-isoindol-2-yl)-3-[4-(2,3-dihydroxy-propyl)-phenyl]-propan-1-one; (R)-3-(4-Allyloxy-phenyl)-2-amino-1-(1,3-dihydro-isoindol-2-yl)-propan-1-one; (E)-3-{4-[(R)-2-Amino-3-(1,3-dihydro-isoindol-2-yl)-3-oxo-propyl]-phenyl}-acrylic acid; (R)-2-Amino-1-(1,3-dihydro-isoindol-2-yl)-3-(3-nitro-phenyl)-propan-1-one; 4′-[(R)-2-Amino-3-(1,3-dihydro-isoindol-2-yl)-3-oxo-propyl]-biphenyl-4-carboxylic acid; (R)-2-Amino-3-(3-chloro-phenyl)-1-(1,3-dihydro-isoindol-2-yl)-propan-1-one; (R)-4-Amino-5-(1,3-dihydro-isoindol-2-yl)-5-oxo-pentanoic acid allyl ester; 4′-[(R)-2-Amino-3-(1,3-dihydro-isoindol-2-yl)-3-oxo-propyl]-biphenyl-3-carboxylic acid; (2R,3S)-2-Amino-1-(1,3-dihydro-isoindol-2-yl)-3-hydroxy-butan-1-one; 2-Amino-1-(1,3-dihydro-isoindol-2-yl)-3-(4-methoxy-2-methyl-phenyl)-propan-1-one; (R)-2-Amino-3-(3,4-dichloro-phenyl)-1-(1,3-dihydro-isoindol-2-yl)-propan-1-one; 2-Amino-3-(4-chloro-2-methoxy-phenyl)-1-(1,3-dihydro-isoindol-2-yl)-propan-1-one; 2-Amino-3-[2-chloro-4-(5-phenyl-pent-1-ynyl)-phenyl]-1-(1,3-dihydro-isoindol-2-yl)-propan-1-one; (R)-2-Amino-3-(2,4-dichloro-phenyl)-1-[5-(2-p-tolyl-ethyl)-1,3-dihydro-isoindol-2-yl]-propan-1-one; (R)-2-Amino-3-(2,4-dichloro-phenyl)-1-[5-((E)-2-p-tolyl-vinyl)-1,3-dihydro-isoindol-2-yl]-propan-1-one; (R)-2-Amino-1-[5-(benzhydryl-amino)-1,3-dihydro-isoindol-2-yl]-3-(2,4-dichloro-phenyl)-propan-1-one, 2-Amino-3-(2-chloro-4-thiophen-2-yl-phenyl)-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-2-(1H-indol-3-yl)-acetamide; 2-Amino-3-(1-benzenesulfonyl-1H-indol-2-yl)-1-(1,3-dihydro-isoindol-2-yl)-propan-1-one; 2-Amino-3-(3-chloro-biphenyl-4-yl)-1-(1,3-dihydro-isoindol-2-yl)-propan-1-one; 2-Amino-3-(2-chloro-4-methoxy-phenyl)-1-(1,3-dihydro-isoindol-2-yl)-propan-1-one; 3-(5-Allyl-naphthalen-1-yl)-2-amino-1-(1,3-dihydro-isoindol-2-yl)-propan-1-one; 2-Amino-1-(1,3-dihydro-isoindol-2-yl)-3-naphthalen-1-yl-propan-1-one; (R)-3-Amino-4-(1,3-dihydro-isoindol-2-yl)-4-oxo-butyric acid allyl ester; (R)-3-Amino-4-(1,3-dihydro-isoindol-2-yl)-4-oxo-butyric acid cyclohexyl ester; (R)-2-Amino-1-(1,3-dihydro-isoindol-2-yl)-3-pyridin-2-yl-propan-1-one; 2-Amino-1-(1,3-dihydro-isoindol-2-yl)-3-(5-phenyl-naphthalen-1-yl)-propan-1-one; 2-Amino-1-(1,3-dihydro-isoindol-2-yl)-3-(4-phenyl-naphthalen-1-yl)-propan-1-one; (R)-2-Amino-3-(2,4-dichloro-phenyl)-1-(1,3-dihydro-isoindol-2-yl)-propan-1-one; 4-[2-Amino-3-(1,3-dihydro-isoindol-2-yl)-3-oxo-propyl]-3-chloro-benzonitrile; Acetic acid 4-[2-amino-3-(1,3-dihydro-isoindol-2-yl)-3-oxo-propyl]-3-chloro-phenyl ester; 2-Amino-3-(3-chloro-3′-methyl-biphenyl-4-yl)-1-(1,3-dihydro-isoindol-2-yl)-propan-1-one; (R)-2-Amino-3-(5-bromo-2-methoxy-phenyl)-1-(1,3-dihydro-isoindol-2-yl)-propan-1-one; 2-Amino-3-[2-chloro-4-((E)-styryl)-phenyl]-1-(1,3-dihydro-isoindol-2-yl)-propan-1-one; (R)-2-Amino-3-[4-(2,6-dichloro-benzyloxy)-phenyl]-1-(1,3-dihydro-isoindol-2-yl)-propan-1-one; 2-Amino-3-[2-chloro-4-(4-phenyl-but-1-ynyl)-phenyl]-1-(1,3-dihydro-isoindol-2-yl)-propan-1-one; 2-Amino-3-[2-chloro-4-(2-methyl-propenyl)-phenyl]-1-(1,3-dihydro-isoindol-2-yl)-propan-1-one; 2-Amino-3-(3,3′-dichloro-biphenyl-4-yl)-1-(1,3-dihydro-isoindol-2-yl)-propan-1-one; 5-[2-Amino-3-(1,3-dihydro-isoindol-2-yl)-3-oxo-propyl]-naphthalene-1-carbonitrile; 2-Amino-3-{2-chloro-4-[(E)-2-(4-chloro-phenyl)-vinyl]-phenyl}-1-(1,3-dihydro-isoindol-2-yl)-propan-1-one; 2-Amino-3-{2-chloro-4-[(E)-2-(4-methoxy-phenyl)-vinyl]-phenyl}-1-(1,3-dihydro-isoindol-2-yl)-propan-1-one; 2-Amino-3-[2-chloro-4-((E)-2-p-tolyl-vinyl)-phenyl]-1-(1,3-dihydro-isoindol-2-yl)-propan-1-one; 2-Amino-3-(2,3-dichloro-phenyl)-1-(1,3-dihydro-isoindol-2-yl)-propan-1-one; 2-Amino-3-(4-bromo-2-chloro-phenyl)-1-(1,3-dihydro-isoindol-2-yl)-propan-1-one; 2-Amino-3-(3-chloro-2′-methyl-biphenyl-4-yl)-1-(1,3-dihydro-isoindol-2-yl)-propan-1-one; 2-Amino-3-[2-chloro-4-(3-phenoxy-prop-1-ynyl)-phenyl]-1-(1,3-dihydro-isoindol-2-yl)-propan-1-one; 2-Amino-3-(2,4-dichloro-3-methyl-phenyl)-1-(1,3-dihydro-isoindol-2-yl)-propan-1-one; 4′-[2-Amino-3-(1,3-dihydro-isoindol-2-yl)-3-oxo-propyl]-3′-chloro-biphenyl-3-carbonitrile; 2-Amino-3-(3-chloro-4′-isopropyl-biphenyl-4-yl)-1-(1,3-dihydro-isoindol-2-yl)-propan-1-one; 2-Amino-3-(3-chloro-2′-methoxy-biphenyl-4-yl)-1-(1,3-dihydro-isoindol-2-yl)-propan-1-one; 2-Amino-3-(3-chloro-2′-fluoro-biphenyl-4-yl)-1-(1,3-dihydro-isoindol-2-yl)-propan-1-one; 2; Amino-1-(1,3-dihydro-isoindol-2-yl)-3-(3,2′,4′-trichloro-biphenyl-4-yl)-propan-1-one; 2-Amino-3-(2-chloro-4-phenylethynyl-phenyl)-1-(1,3-dihydro-isoindol-2-yl)-propan-1-one; 2-Amino-3-(2-chloro-4-methyl-phenyl)-1-(1,3-dihydro-isoindol-2-yl)-propan-1-one; 2-Amino-3-(2-chloro-4-hydroxy-phenyl)-1-(1,3-dihydro-isoindol-2-yl)-propan-1-one; 2-Amino-3-(3,4′-dichloro-biphenyl-4-yl)-1-(1,3-dihydro-isoindol-2-yl)-propan-1-one; 2-Amino-3-(3-chloro-4′-methoxy-biphenyl-4-yl)-1-(1,3-dihydro-isoindol-2-yl)-propan-1-one; 2-Amino-3-(3-chloro-4′-methyl-biphenyl-4-yl)-1-(1,3-dihydro-isoindol-2-yl)-propan-1-one; (R)-2-Amino-1-[5-(2-chloro-phenyl)-1,3-dihydro-isoindol-2-yl]-3-(2,4-dichloro-phenyl)-propan-1-one; 2-Amino-3-(6-chloro-benzo[1,3]dioxol-5-yl)-1-(1,3-dihydro-isoindol-2-yl)-propan-1-one; (R)-2-Amino-1-[5-(2,4-dichloro-benzylamino)-1,3-dihydro-isoindol-2-yl]-3-(2,4-dichloro-phenyl)-propan-1-one; (R)-2-Amino-3-(2,4-dichloro-phenyl)-1-{5-[(pyridin-4-ylmethyl)-amino]-1,3-dihydro-isoindol-2-yl}-propan-1-one; 1-{2-[(R)-2-Amino-3-(2,4-dichloro-phenyl)-propionyl]-2,3-dihydro-1H-isoindol-5-yl}-3-(3,5-dichloro-phenyl)-urea; 1-{2-[(R)-2-Amino-3-(2,4-dichloro-phenyl)-propionyl]-2,3-dihydro-1H-isoindol-5-yl}-3-isopropyl-urea; 1-{2-[(R)-2-Amino-3-(2,4-dichloro-phenyl)-propionyl]-2,3-dihydro-1H-isoindol-5-ylmethyl}-3-(4-dimethylamino-phenyl)-urea; 1-{2-[(R)-2-Amino-3-(2,4-dichloro-phenyl)-propionyl]-2,3-dihydro-1H-isoindol-5-yl}-3-benzyl-urea; (R)-2-Amino-3-(2,4-dichloro-phenyl)-1-(5-naphthalen-1-yl-1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-acetamide; (R)-2-Amino-3-(2,4-dichloro-phenyl)-1-[5-(4-methoxy-benzylamino)-1,3-dihydro-isoindol-2-yl]-propan-1-one; N-{4-[2-Amino-3-(1,3-dihydro-isoindol-2-yl)-3-oxo-propyl]-3-chloro-phenyl}-methanesulfonamide; N-{2-[(R)-2-Amino-3-(2,4-dichloro-phenyl)-propionyl]-2,3-dihydro-1H-isoindol-5-yl}-propionamide; 1-{2-[(R)-2-Amino-3-(2,4-dichloro-phenyl)-propionyl]-2,3-dihydro-1H-isoindol-5-yl}-3-phenyl-urea; 1-{2-[(R)-2-Amino-3-(2,4-dichloro-phenyl)-propionyl]-2,3-dihydro-1H-isoindol-5-yl}-3-(3-benzyl-phenyl)-urea; (E)-3-{5-[(R)-2-Amino-3-(1,3-dihydro-isoindol-2-yl)-3-oxo-propyl]-thiophen-2-yl}-acrylic acid methyl ester; (E)-4-{5-[(R)-2-Amino-3-(1,3-dihydro-isoindol-2-yl)-3-oxo-propyl]-thiophen-2-yl}-but-2-enoic acid methyl ester; (R)-2-Amino-1-[5-(cyclohexylmethyl-amino)-1,3-dihydro-isoindol-2-yl]-3-(2,4-dichloro-phenyl)-propan-1-one; 1-{2-[(R)-2-Amino-3-(2,4-dichloro-phenyl)-propionyl]-2,3-dihydro-1H-isoindol-5-yl}-3-(3-phenoxy-phenyl)-urea; 1-{2-[(R)-2-Amino-3-(2,4-dichloro-phenyl)-propionyl]-2,3-dihydro-1H-isoindol-5-yl}-3-(4′-methyl-biphenyl-4-yl)-urea; (R)-2-Amino-1-(5,6-dichloro-1,3-dihydro-isoindol-2-yl)-3-(2,4-dichloro-phenyl)-propan-1-one; (R)-2-Amino-1-[5-(3-chloro-phenyl)-1,3-dihydro-isoindol-2-yl]-3-(2,4-dichloro-phenyl)-propan-1-one; (R)-2-Amino-3-(2,4-dichloro-phenyl)-1-[5-(3-methoxy-phenyl)-1,3-dihydro-isoindol-2-yl]-propan-1-one; 4-{5-[(R)-2-Amino-3-(1,3-dihydro-isoindol-2-yl)-3-oxo-propyl]-thiophen-2-yl}-benzoic acid methyl ester; 3-Methyl-but-2-enoic acid {2-[(R)-2-amino-3-(2,4-dichloro-phenyl)-propionyl]-2,3-dihydro-1H-isoindol-5-yl}-amide; 1-{2-[(R)-2-Amino-3-(2,4-dichloro-phenyl)-propionyl]-2,3-dihydro-1H-isoindol-5-yl}-3-(2,4-dichloro-phenyl)-urea; N-{2-[(R)-2-Amino-3-(3-chloro-phenyl)-propionyl]-2,3-dihydro-1H-isoindol-5-yl}-benzamide; 2-Amino-3-(2-chloro-4-methanesulfonyl-phenyl)-1-(1,3-dihydro-isoindol-2-yl)-propan-1-one; 3-(4-Allyl-2-chloro-phenyl)-2-amino-1-(1,3-dihydro-isoindol-2-yl)-propan-1-one; 2-Amino-1-(1,3-dihydro-isoindol-2-yl)-3-quinolin-7-yl-propan-1-one; 2-Amino-3-(3,2′-dichloro-biphenyl-4-yl)-1-(1,3-dihydro-isoindol-2-yl)-propan-1-one; 2-Amino-3-(3-chloro-3′-methoxy-biphenyl-4-yl)-1-(1,3-dihydro-isoindol-2-yl)-propan-1-one; 2-Amino-3-(4-benzyloxy-2-chloro-phenyl)-1-(1,3-dihydro-isoindol-2-yl)-propan-1-one; 2-Amino-3-{2-chloro-4-[(E)-2-(3-fluoro-phenyl)-vinyl]-phenyl}-1-(1,3-dihydro-isoindol-2-yl)-propan-1-one; 2-Amino-3-{2-chloro-4-[(E)-2-(4-fluoro-phenyl)-vinyl]-phenyl}-1-(1,3-dihydro-isoindol-2-yl)-propan-1-one; 2-Amino-3-(2-chloro-3,4-dimethoxy-phenyl)-1-(1,3-dihydro-isoindol-2-yl)-propan-1-one; 2-Amino-3-[2-chloro-4-((E)-2-cyclohexyl-vinyl)-phenyl]-1-(1,3-dihydro-isoindol-2-yl)-propan-1-one; 2-Amino-3-(2,4-dichloro-6-hydroxy-phenyl)-1-(1,3-dihydro-isoindol-2-yl)-propan-1-one; 2-Amino-3-(3-chloro-4′-methoxy-3′-methyl-biphenyl-4-yl)-1-(1,3-dihydro-isoindol-2-yl)-propan-1-one; (R)-2-Amino-3-(2,4-dichloro-phenyl)-1-[5-((E)-styryl)-1,3-dihydro-isoindol-2-yl]-propan-1-one; 2-Amino-3-(2,4-dichloro-5-fluoro-phenyl)-1-(1,3-dihydro-isoindol-2-yl)-propan-1-one; 2-Amino-3-(1-chloro-naphthalen-2-yl)-1-(1,3-dihydro-isoindol-2-yl)-propan-1-one; 2-Amino-3-(3-chloro-2′,5′-dimethoxy-biphenyl-4-yl)-1-(1,3-dihydro-isoindol-2-yl)-propan-1-one; 2-Amino-3-(3-bromo-2,4-dichloro-phenyl)-1-(1,3-dihydro-isoindol-2-yl)-propan-1-one; (R)-2-Amino-1-(5-benzylamino-1,3-dihydro-isoindol-2-yl)-3-(2,4-dichloro-phenyl)-propan-1-one; 2-Amino-3-(2,4-dichloro-6-methoxy-phenyl)-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-ylmethyl}-isobutyramide; (S)-2-Amino-N-{2-[(R)-2-amino-3-(2,4-dichloro-phenyl)-propionyl]-2,3-dihydro-1H-isoindol-5-yl}-3-(2,4-dichloro-phenyl)-propionamide; 1-{2-[(R)-2-Amino-3-(2,4-dichloro-phenyl)-propionyl]-2,3-dihydro-1H-isoindol-5-yl}-3-p-tolyl-urea; 1-{2-[(R)-2-Amino-3-(2,4-dichloro-phenyl)-propionyl]-2,3-dihydro-1H-isoindol-5-yl}-3-(4-phenoxy-phenyl)-urea; 1-{2-[(R)-2-Amino-3-(2,4-dichloro-phenyl)-propionyl]-2,3-dihydro-1H-isoindol-5-yl}-3-biphenyl-4-yl-urea; N-{2-[(R)-2-Amino-3-(2,4-dichloro-phenyl)-propionyl]-2,3-dihydro-1H-isoindol-5-yl}-isobutyramide; ({2-[(R)-2-Amino-3-(2,4-dichloro-phenyl)-propionyl]-2,3-dihydro-1H-isoindol-5-yl}-methoxyoxalyl-amino)-oxo-acetic acid methyl ester; 1-{2-[(R)-2-Amino-3-(2,4-dichloro-phenyl)-propionyl]-2,3-dihydro-1H-isoindol-5-yl}-3-(4-dimethylamino-phenyl)-urea; 2-[(R)-2-Amino-3-(2,4-dichloro-phenyl)-propionyl]-2,3-dihydro-1H-isoindole-5-carbonitrile; (R)-2-Amino-1-(5-aminomethyl-1,3-dihydro-isoindol-2-yl)-3-(2,4-dichloro-phenyl)-propan-1-one; N-{2-[(R)-2-Amino-3-(2,4-dichloro-phenyl)-propionyl]-2,3-dihydro-1H-isoindol-5-ylmethyl}-acetamide; 2-Amino-3-(2,5-dichloro-phenyl)-1-(1,3-dihydro-isoindol-2-yl)-propan-1-one; 3-(4′-Acetyl-3-chloro-biphenyl-4-yl)-2-amino-1-(1,3-dihydro-isoindol-2-yl)-propan-1-one; 4′-[2-Amino-3-(1,3-dihydro-isoindol-2-yl)-3-oxo-propyl]-3′-chloro-biphenyl-4-carbonitrile; 2-Amino-3-(5-bromo-naphthalen-1-yl)-1-(1,3-dihydro-isoindol-2-yl)-propan-1-one; 2-Amino-3-(4-bromo-naphthalen-1-yl)-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-oxalamic acid methyl ester; (R)-2-Amino-3-(2,4-dichloro-phenyl)-1-{5-[(pyridin-3-ylmethyl)-amino]-1,3-dihydro-isoindol-2-yl}-propan-1-one; 1-{2-[(R)-2-Amino-3-(2,4-dichloro-phenyl)-propionyl]-2,3-dihydro-1H-isoindol-5-yl}-3-(3,5-dimethoxy-phenyl)-urea; (S)-2-Acetylamino-N-{2-[(R)-2-amino-3-(2,4-dichloro-phenyl)-propionyl]-2,3-dihydro-1H-isoindol-5-yl}-3-(1H-indol-3-yl)-propionamide; 2-Amino-1-(1,3-dihydro-isoindol-2-yl)-3-(1H-indol-6-yl)-propan-1-one; (R)-2-Amino-3-[5-(2-chloro-phenyl)-thiophen-2-yl]-1-(1,3-dihydro-isoindol-2-yl)-propan-1-one; (R)-2-Amino-1-(5-bromo-1,3-dihydro-isoindol-2-yl)-3-(2,4-dichloro-phenyl)-propan-1-one; (R)-2-Amino-3-(2,4-dichloro-phenyl)-1-(5-nitro-1,3-dihydro-isoindol-2-yl)-propan-1-one; (R)-2-Amino-3-(2,4-dichloro-phenyl)-1-(5-fluoro-1,3-dihydro-isoindol-2-yl)-propan-1-one; (R)-2-Amino-3-(2,4-dichloro-phenyl)-1-(5-methyl-1,3-dihydro-isoindol-2-yl)-propan-1-one; 3-{2-[(R)-2-Amino-3-(2,4-dichloro-phenyl)-propionyl]-2,3-dihydro-1H-isoindol-5-yl}-benzonitrile; N-{2-[(R)-2-Amino-3-(2,4-dichloro-phenyl)-propionyl]-2,3-dihydro-1H-isoindol-5-ylmethyl}-2-methyl-butyramide; 3-(4-Allyloxy-2-chloro-phenyl)-2-amino-1-(1,3-dihydro-isoindol-2-yl)-propan-1-one; 3-(3′-Acetyl-3-chloro-biphenyl-4-yl)-2-amino-1-(1,3-dihydro-isoindol-2-yl)-propan-1-one; 2-Amino-3-(3,3′-dichloro-4′-fluoro-biphenyl-4-yl)-1-(1,3-dihydro-isoindol-2-yl)-propan-1-one; 2-Amino-3-[5-(2-chloro-phenyl)-naphthalen-1-yl]-1-(1,3-dihydro-isoindol-2-yl)-propan-1-one; 2-Amino-3-[4-(2-chloro-phenyl)-naphthalen-1-yl]-1-(1,3-dihydro-isoindol-2-yl)-propan-1-one; (R)-2-Amino-3-[5-(4-chloro-phenyl)-thiophen-2-yl]-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-2-phenyl-butyramide; N-{2-[(R)-2-Amino-3-(2,4-dichloro-phenyl)-propionyl]-2,3-dihydro-1H-isoindol-5-yl}-3-methyl-butyramide; N-{2-[(R)-2-Amino-3-(2,4-dichloro-phenyl)-propionyl]-2,3-dihydro-1H-isoindol-5-yl}-4-trifluoromethyl-benzamide; 3-{2-[(R)-2-Amino-3-(2,4-dichloro-phenyl)-propionyl]-2,3-dihydro-1H-isoindol-5-yl}-N,N-dimethyl-benzamide; (R)-2-Amino-3-(2,4-dichloro-phenyl)-1-(5-naphthalen-2-yl-1,3-dihydro-isoindol-2-yl)-propan-1-one; (R)-2-Amino-3-(2,4-dichloro-phenyl)-1-[5-(2-methoxy-phenyl)-1,3-dihydro-isoindol-2-yl]-propan-1-one; N-{2-[(R)-2-Amino-3-(2,4-dichloro-phenyl)-propionyl]-2,3-dihydro-1H-isoindol-5-ylmethyl}-oxalamic acid methyl ester; 1-{2-[(R)-2-Amino-3-(2,4-dichloro-phenyl)-propionyl]-2,3-dihydro-1H-isoindol-5-ylmethyl}-3-isopropyl-urea; (R)-2-Amino-3-[5-(3-chloro-phenyl)-thiophen-2-yl]-1-(1,3-dihydro-isoindol-2-yl)-propan-1-one; 3-{5-[(R)-2-Amino-3-(1,3-dihydro-isoindol-2-yl)-3-oxo-propyl]-thiophen-2-yl}-benzoic acid; N-{2-[(R)-2-Amino-3-(2,4-dichloro-phenyl)-propionyl]-2,3-dihydro-1H-isoindol-5-yl}-2-methyl-butyramide; 1-{2-[(R)-2-Amino-3-(2,4-dichloro-phenyl)-propionyl]-2,3-dihydro-1H-isoindol-5-yl}-3-(2,5-dimethoxy-phenyl)-urea; (R)-2-Amino-3-[5-(2,4-dichloro-phenyl)-thiophen-2-yl]-1-(1,3-dihydro-isoindol-2-yl)-propan-1-one; (R)-2-Amino-3-(5-bromo-thiophen-2-yl)-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-benzamide; (E)-3-{5-[(R)-2-Amino-3-(1,3-dihydro-isoindol-2-yl)-3-oxo-propyl]-thiophen-2-yl}-acrylic acid; N-{2-[(R)-2-Amino-3-(2,4-dichloro-phenyl)-propionyl]-2,3-dihydro-1H-isoindol-5-yl}-2-(4-dimethylamino-phenyl)-acetamide; (R)-2-Amino-3-(2,4-dichloro-phenyl)-1-(5-isobutylamino-1,3-dihydro-isoindol-2-yl)-propan-1-one; (R)-2-Amino-1-(5-dimethylsulfonamyl-amido-1,3-dihydro-isoindol-2-yl)-3-(2,4-dichloro-phenyl)-propan-1-one; 1-{2-[(R)-2-Amino-3-(2,4-dichloro-phenyl)-propionyl]-2,3-dihydro-1H-isoindol-5-yl}-3-(3,4,5-trimethoxy-phenyl)-urea; 2-Amino-3-(2-chloro-4-thiophen-3-ylethynyl-phenyl)-1-(1,3-dihydro-isoindol-2-yl)-propan-1-one; (R)-2-Amino-3-(2,4-dichloro-phenyl)-1-[5-(4-pyridin-4-yl-benzylamino)-1,3-dihydro-isoindol-2-yl]-propan-1-one; 2-Amino-3-(2,3-dihydro-1H-indol-6-yl)-1-(1,3-dihydro-isoindol-2-yl)-propan-1-one; 3-{5-[(R)-2-Amino-3-(1,3-dihydro-isoindol-2-yl)-3-oxo-propyl]-thiophen-2-yl}-benzoic acid methyl ester; N-{2-[(R)-2-Amino-3-(2,4-dichloro-phenyl)-propionyl]-2,3-dihydro-1H-isoindol-5-yl}-2,5-difluoro-benzamide; 1-(4-Acetyl-phenyl)-3-{2-[(R)-2-amino-3-(2,4-dichloro-phenyl)-propionyl]-2,3-dihydro-1H-isoindol-5-yl}-urea; (R)-2-Amino-1-(5-bis-methylsulfon-amido-1,3-dihydro-isoindol-2-yl)-3-(2,4-dichloro-phenyl)-propan-1-one; N-{2-[(R)-2-Amino-3-(2,4-dichloro-phenyl)-propionyl]-2,3-dihydro-1H-isoindol-5-yl}-3,3-dimethyl-butyramide; 1-{2-[(R)-2-Amino-3-(2,4-dichloro-phenyl)-propionyl]-2,3-dihydro-1H-isoindol-5-yl}-3,3-bis(3,5-dimethoxy-phenyl)-urea; N-{2-[(R)-2-Amino-3-(2,4-dichloro-phenyl)-propionyl]-2,3-dihydro-1H-isoindol-5-yl}-4-dimethylamino-benzamide; Cyclopentanecarboxylic acid {2-[(R)-2-amino-3-(2,4-dichloro-phenyl)-propionyl]-2,3-dihydro-1H-isoindol-5-yl}-amide; 1-{2-[(R)-2-Amino-3-(2,4-dichloro-phenyl)-propionyl]-2,3-dihydro-1H-isoindol-5-yl}-3-benzoyl-urea; (R)-3-(5-Allyl-thiophen-2-yl)-2-amino-1-(1,3-dihydro-isoindol-2-yl)-propan-1-one; (R)-2-Amino-1-(5-amino-1,3-dihydro-isoindol-2-yl)-3-(2,4-dichloro-phenyl)-propan-1-one; (R)-2-Amino-3-[5-(2-bromo-phenyl)-thiophen-2-yl]-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-3-trifluoromethyl-benzamide; Morpholine-4-carboxylic acid {2-[(R)-2-amino-3-(2,4-dichloro-phenyl)-propionyl]-2,3-dihydro-1H-isoindol-5-yl}-amide; 2-Amino-3-(4-benzylamino-2-chloro-phenyl)-1-(1,3-dihydro-isoindol-2-yl)-propan-1-one; 2-Amino-3-(2-chloro-4-dimethylamino-phenyl)-1-(1,3-dihydro-isoindol-2-yl)-propan-1-one; 2-Amino-3-(3-chloro-2′,4′-dimethyl-biphenyl-4-yl)-1-(1,3-dihydro-isoindol-2-yl)-propan-1-one; 2-Amino-3-(3-chloro-3′,4′-dimethoxy-biphenyl-4-yl)-1-(1,3-dihydro-isoindol-2-yl)-propan-1-one; 4-{5-[(R)-2-Amino-3-(1,3-dihydro-isoindol-2-yl)-3-oxo-propyl]-thiophen-2-yl}-benzoic acid; N-{2-[(R)-2-Amino-3-(2,4-dichloro-phenyl)-propionyl]-2,3-dihydro-1H-isoindol-5-yl}-nicotinamide; (R)-2-Amino-3-(2,4-dichloro-phenyl)-1-(1,3-dihydro-benzo[f]isoindol-2-yl)-propan-1-one; N-{2-[(R)-2-Amino-3-(2,4-dichloro-phenyl)-propionyl]-2,3-dihydro-1H-isoindol-5-yl}-2,2,2-trifluoro-acetamide; 1-{2-[(R)-2-Amino-3-(2,4-dichloro-phenyl)-propionyl]-2,3-dihydro-1H-isoindol-5-yl}-3-((S)-1-phenyl-ethyl)-urea; (R)-2-Amino-3-(2,4-dichloro-phenyl)-1-(5-phenyl-1,3-dihydro-isoindol-2-yl)-propan-1-one; (R)-2-Amino-1-(5-biphenyl-3-yl-1,3-dihydro-isoindol-2-yl)-3-(2,4-dichloro-phenyl)-propan-1-one; (R)-2-Amino-1-(1,3-dihydro-isoindol-2-yl)-3-thiophen-2-yl-propan-1-one; 2-Amino-3-{2-chloro-4-[(E)-2-(4-trifluoromethyl-phenyl)-vinyl]-phenyl}-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-bis(4-methyl-benzene)-sulfonamide; (R)-2-Amino-3-(5-bromo-thiophen-2-yl)-1-(5-fluoro-1,3-dihydro-isoindol-2-yl)-propan-1-one; 2-Amino-1-(1,3-dihydro-isoindol-2-yl)-3-(1H-indol-2-yl)-propan-1-one; (1-Amino-indan-1-yl)-(1,3-dihydro-isoindol-2-yl)-methanone; (R)-2-Amino-1-(5-benzyl-1,3-dihydro-isoindol-2-yl)-3-(2,4-dichloro-phenyl)-propan-1-one; N-{2-[(R)-2-Amino-3-(2,4-dichloro-phenyl)-propionyl]-2,3-dihydro-1H-isoindol-5-yl}-5-chloro-2-trifluoromethyl-benzamide; N-{2-[(R)-2-Amino-3-(2,4-dichloro-phenyl)-propionyl]-2,3-dihydro-1H-isoindol-5-yl}-methanesulfonamide; N-{2-[(R)-2-Amino-3-(2,4-dichloro-phenyl)-propionyl]-2,3-dihydro-1H-isoindol-5-yl}-5-fluoro-2-trifluoromethyl-benzamide; 2-Amino-3-(3-chloro-4′-methoxy-2′-methyl-biphenyl-4-yl)-1-(1,3-dihydro-isoindol-2-yl)-propan-1-one; 2-Amino-3-(2-chloro-4-trimethylsilanylethynyl-phenyl)-1-(1,3-dihydro-isoindol-2-yl)-propan-1-one; (R)-2-Amino-1-(1,3-dihydro-isoindol-2-yl)-3-[5-(2-trifluoromethyl-phenyl)-thiophen-2-yl]-propan-1-one; (S)-2-Amino-3-(2,5-dibromo-thiophen-3-yl)-1-(1,3-dihydro-isoindol-2-yl)-propan-1-one; 2-Amino-3-[2-chloro-4-(3-methyl-3H-imidazol-4-ylethynyl)-phenyl]-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-4-methoxy-benzenesulfonamide; (R)-2-Amino-1-(5-bromo-1,3-dihydro-isoindol-2-yl)-3-(5-bromo-thiophen-2-yl)-propan-1-one; N-{2-[(R)-2-Amino-3-(2,4-dichloro-phenyl)-propionyl]-2,3-dihydro-1H-isoindol-5-yl}-bis(4-methoxy-benzene)sulfonamide; 2-Amino-3-(4-benzofuran-2-yl-2-chloro-phenyl)-1-(1,3-dihydro-isoindol-2-yl)-propan-1-one; [(S)-1-{2-[(R)-2-Amino-3-(2,4-dichloro-phenyl)-propionyl]-2,3-dihydro-1H-isoindol-5-ylcarbamoyl}-2-(1H-indol-3-yl)-ethyl]-carbamic acid benzyl ester; (R)-2-Amino-3-benzo[b]thiophen-3-yl-1-(1,3-dihydro-isoindol-2-yl)-propan-1-one; N-{2-[(R)-2-Amino-3-(2,4-dichloro-phenyl)-propionyl]-2,3-dihydro-1H-isoindol-5-yl}-4-methyl-benzenesulfonamide; N-{2-[(R)-2-Amino-3-(5-bromo-thiophen-2-yl)-propionyl]-2,3-dihydro-1H-isoindol-5-yl}-benzamide; (R)-2-Amino-1-(1,3-dihydro-isoindol-2-yl)-propan-1-one; (R)-2-Amino-1-[5-(1H-benzoimidazol-2-ylamino)-1,3-dihydro-isoindol-2-yl]-3-(2,4-dichloro-phenyl)-propan-1-one; (R)-2-Amino-1-[5-(benzooxazol-2-ylamino)-1,3-dihydro-isoindol-2-yl]-3-(2,4-dichloro-phenyl)-propan-1-one; (R)-2-Amino-1-(1,3-dihydro-isoindol-2-yl)-3-methyl-butan-1-one; (R)-2-Amino-3-(2,4-dichloro-phenyl)-1-(2,6-dihydro-4H-pyrrolo[3,4-c]pyrazol-5-yl)-propan-1-one; (R)-2-Amino-3-(5-bromo-thiophen-2-yl)-1-(2,6-dihydro-4H-pyrrolo[3,4-c]pyrazol-5-yl)-propan-1-one; (R)-2-Amino-3-(2,4-dichloro-phenyl)-1-(5,7-dihydro-pyrrolo[3,4-b]pyridin-6-yl)-propan-1-one; (R)-2-Amino-3-(2,4-dichloro-phenyl)-1-(5,7-dihydro-pyrrolo[3,4-d]pyrimidin-6-yl)-propan-1-one; (R)-2-Amino-3-(5-bromo-thiophen-2-yl)-1-(5,7-dihydro-pyrrolo[3,4-d]pyrimidin-6-yl)-propan-1-one; (R)-2-Amino-3-(5-bromo-thiophen-2-yl)-1-(5,7-dihydro-pyrrolo[3,4-b]pyridin-6-yl)-propan-1-one or pharmaceutically acceptable salts of these compounds.

In one embodiment is the use of a compound according to the invention in the preparation of a pharmaceutical composition.

In another embodiment is a pharmaceutical composition comprising a compound according to the above.

In certain embodiments, the pharmaceutical composition has a compound according to the above and an acceptable pharmaceutical carrier.

Another embodiment provides use of the composition in the manufacture of a medicament to treat a proliferative or hyperproliferative disease, a HDAC-dependent disease, or a disease responsive to inhibition of HDAC activity.

In another embodiment, there is provided the use of a compound according to the above in the preparation of a pharmaceutical composition for use in the treatment of a HDAC dependent disease.

Compounds of the invention may be used in the treatment of HDAC dependent diseases or for the manufacture of pharmaceutical compositions for use in the treatment of these diseases, methods of use of compounds of the present invention in the treatment of these diseases, or pharmaceutical preparations having compounds of the present invention for the treatment of these diseases.

The present invention also relates to a method of treating HDAC dependent diseases comprising administering compounds of the present invention to a warm-blooded animal, including, for example, a human. The present invention also relates to pharmaceutical preparations having compounds of the present invention for the treatment of a HDAC dependent disease, novel aminoalkyl compounds, a process for the manufacture of the aminoalkyl compounds of the present invention, and novel starting materials and intermediates for their manufacture. The present invention also relates to use of a compound of the present invention in the manufacture of a pharmaceutical preparation for the treatment of a HDAC dependent disease.

As appropriate, unsubstituted means that there is no substituent or that the only substituents are hydrogen.

Halo substituents are selected from fluoro, chloro, bromo and iodo, preferably fluoro or chloro.

A hetero modified substituent (alternatively referred to as being heterosubstituted) is a substituent that includes ones or more heteroatoms selected from nitrogen (N), sulfur (S) and oxygen (O).

Alkyl substituents include straight and branched C₁-C₁₀ alkyl, unless otherwise noted. Examples of suitable straight and branched C₁-C₁₀ alkyl substituents include methyl, ethyl, n-propyl, 2-propyl, n-butyl, sec-butyl, t-butyl, and the like. Unless otherwise noted, the alkyl substituents include both unsubstituted alkyl groups and alkyl groups that are substituted by one or more suitable substituents, including unsaturation (i.e., there are one or more double or triple C—C bonds), acyl, cycloalkyl, halo, oxyalkyl, alkylamino, aminoalkyl, acylamino and alkoxy. Preferred substituents for alkyl groups include halo, hydroxy, alkoxy, oxyalkyl, alkylamino, and aminoalkyl.

Cycloalkyl substituents include C₃-C₉ cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆ alkyl, halo, hydroxy, aminoalkyl, oxyalkyl, alkylamino, and alkoxy, or are heterosubstituted. Other substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

Heterocycloalkyl substituents include 3 to 9 membered aliphatic rings, such as 4 to 7 membered aliphatic rings, containing one or more heteroatoms, such as one to three heteroatoms selected from nitrogen, sulfur and oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. Unless otherwise noted, the rings are unsubstituted or substituted on the carbon atoms by one or more suitable substituents, including C₁-C₆ alkyl, C₄-C₉ cycloalkyl, aryl, heteroaryl, arylalkyl (e.g., benzyl), and heteroarylalkyl (e.g., pyridylmethyl), halo, amino, alkyl amino and alkoxy. Unless otherwise noted, nitrogen heteroatoms are unsubstituted or substituted by H, C₁-C₄ alkyl, arylalkyl (e.g., benzyl), and heteroarylalkyl (e.g., pyridylmethyl), acyl, aminoacyl, alkylsulfonyl, and arylsulfonyl.

Cycloalkylalkyl substituents include compounds of the formula —(CH₂)ₙ-cycloalkyl wherein n is a number from 1-6. Suitable cycloalkylalkyl substituents include cyclopentylmethyl-, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents, including C₁-C₆ alkyl, cycloalkylalkyl (e.g., cyclopropylmethyl), O(CO)alkyl, oxyalkyl, halo, nitro, amino, alkylamino, aminoalkyl, alkyl ketones, nitrile, carboxyalkyl, alkylsulfonyl, aminosulfonyl, arylsulfonyl, and alkoxy. Preferred substituents include including C₁-C₆ alkyl, cycloalkyl (e.g., cyclopropylmethyl), alkoxy, oxyalkyl, halo, nitro, amino, alkylamino, aminoalkyl, alkyl ketones, nitrile, carboxyalkyl, alkylsulfonyl, arylsulfonyl, and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents, including, e.g., C₁-C₆ alkyl, cycloalkylalkyl (e.g., cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and alkoxy.

Heteroaryl substituents include compounds with a 5 to 7 member aromatic ring containing one or more heteroatoms, for example from 1 to 4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted. Useful N substituents include H, C₁-C₄ alkyl, acyl, aminoacyl, and sulfonyl.

Alkylaryl substituents, referred to alternatively as arylalkyl substituents, include alkyl and aryl portions. Alkylaryl groups may be attached to the chemical backbone via either the alkyl or the aryl portion of the substituent. arylalkyl substituents include groups of the formula —(CH₂)ₙ-aryl, —(CH₂)ₙ₋₁—(CHaryl)-(CH₂)ₙ-aryl or —(CH₂)ₙ₋₁CH(aryl)(aryl) wherein aryl and n are as defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. arylalkyl substituents are unsubstituted or substituted in the alkyl moiety or the aryl moiety or both as described above for alkyl and aryl substituents, and include straight or branched chain alkyl substituents attached to aryl substituents, which may be further substituted by alkyl or cycloalkyl substituents.

Heteroarylalkyl substituents, alternatively referred to as heterosubstituted arylalkyl substituents, include groups of the formula —(CH₂)ₙ-heteroaryl wherein heteroaryl and n are as defined above and the bridging group is linked to a carbon or a nitrogen of the heteroaryl portion, such as 2-, 3- or 4-pyridylmethyl, imidazolylmethyl, quinolylethyl, and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NRR′)—(CH₂), —R₃ wherein n is an integer between 1 and 5, and R, R′ and R₃ are as described above. Suitable aminoacyl substituents include natural and non-natural amino acids such as glycinyl, D-tryptophanyl, L-lysinyl, D-homoserinyl, L-homoserinyl, 4-aminobutryic acyl, any of which may optionally contain -3-amin-4-hexenoyl.

R and R′ are the same or are different and may be H or are any aliphatic, aryl, heteroaryl, alkylaryl or heteroalkylaryl moiety as defined above.

Non-aromatic polycycle substituents include bicyclic and tricyclic fused ring systems where each ring can be 4-9 membered and each ring can contain zero, 1 or more double and/or triple bonds. Suitable examples of non-aromatic polycycles include decalin, perhydrobenzocycloheptene, octahydroindene, perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4-9 membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene, 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatom, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted. Useful N substituents include H, C₁-C₄ alkyl, acyl, aminoacyl, and sulfonyl.

Non-aromatic polyheterocyclic substituents include bicyclic and tricyclic fused ring systems where each ring can be 4-9 membered, contain one or more heteroatom, for example, 1, 2, 3, or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted. Useful N substituents include H, C₁-C₄ alkyl, acyl, aminoacyl, and sulfonyl.

Mixed aryl and non-aryl polyheterocycles substituents include bicyclic and tricyclic fused ring systems where each ring can be 4-9 membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzo-diazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents, including, —N—OH, ═N—OH, alkyl and the alkyl substituents identified above. Nitrogen atoms are unsubstituted or substituted. Useful N substituents include H, C₁-C₄ alkyl, acyl, aminoacyl, and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, for example methane sulfonyl, benzene sulfonyl, tosyl and the like.

The general terms used herein before and hereinafter have within the context of this disclosure the following meanings, unless otherwise indicated:

“Aryl” is an aromatic radical having 6 to 14 carbon atoms, for example, phenyl, naphthyl, indenyl, azulenyl, or anthryl, and is unsubstituted or substituted by one or more, wherein the substituents are selected from any of the functional groups defined below, and including: lower halo, alkyl, substituted alkyl, halo lower alkyl e.g., trifluoromethyl, lower alkenyl, lower alkynyl, lower alkanoyl, lower alkoxy, hydroxy, another aryl, etherified or esterified hydroxy, amino, mono- or disubstituted amino, amino lower alkyl, amino lower alkoxy; acetyl amino; amidino, halogen, nitro, cyano, cyano lower alkyl, carboxy, esterified carboxy, lower alkoxy carbonyl, e.g., methoxy carbonyl, n-propoxy carbonyl or iso-propoxy carbonyl, alkanoyl, benzoyl, carbamoyl, N-mono- or N,N-disubstituted carbamoyl, carbamates, alkyl carbamic acid esters, amidino, guanidino, urea, ureido, mercapto, sulfo, lower alkylthio, sulfoamino, sulfonamide, benzosulfonamide, sulfonate, phenyl, benzyl, phenoxy, benzyloxy, phenylthio, phenyl-lower alkylthio, alkylphenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as especially trifluoromethane sulfonyl, dihydroxybora (—B(OH)₂), heterocyclyl, and lower alkylene dioxy bound at adjacent C-atoms of the ring, such as methylene dioxy, phosphono (—P(═O)(OH)₂), hydroxy-lower alkoxy phosphoryl or di-lower alkoxyphosphoryl, carbamoyl, mono- or di-lower alkylcarbamoyl, mono- or di-(hydroxy-lower alkyl)-carbamoyl, or —NR₁₄R₁₅, wherein R₁₄ and R₁₅ can be the same or different and are independently H; lower alkyl (e.g., methyl, ethyl or propyl); or R₁₄ and R₁₅ together with the N atom form a 3- to 8-membered heterocyclic ring containing 1-4 nitrogen, oxygen or sulfur atoms (e.g., piperazinyl, lower alkyl-piperazinyl, azetidinyl, pyrrolidinyl, piperidino, morpholinyl, imidazolinyl).

Aryl is, for example, phenyl that is either unsubstituted or independently substituted by one or two substituents selected from a solubilizing group selected from the group consisting of: halo (such as Cl, Br or F); hydroxy; lower alkyl (such as C₁-C₃ lower alkyl such as methyl); aryl (such as phenyl or benzyl); amino; amino lower alkyl (such as dimethylamino); acetyl amino; amino lower alkoxy (such as ethoxyamine); substituted lower alkyl (such as fluoror ethyl); alkoxy (such as methoxy or benzyloxy where the benzyl ring may be substituted or unsubstituted, such as 3,4-dichlorobenzyloxy); sulfoamino; substituted or unsubstituted sulfonamide (such as benzo sulfonamide, chlorobenzene sulfonamide or 2,3-dichloro benzene sulfonamide); substituted or unsubstituted sulfonate (such as chloro-phenyl sulfonate); substituted urea (such as 3-trifluoro-methyl-phenyl urea or 4-morpholin-4-yl-3-trifluoromethyl-phenyl-urea); alkyl carbamic acid ester or carbamates (such as ethyl-N-phenyl-carbamate) or —NR₁₄R₁₅, wherein R₁₄ and R₁₅ can be the same or different and are independently H; lower alkyl (e.g., methyl, ethyl or propyl); or R₁₄ and R₁₅ together with the N atom form a 3- to 8-membered heterocyclic ring containing 1-4 nitrogen, oxygen or sulfur atoms (e.g., piperazinyl, lower alkyl-piperazinyl, pyridyl, indolyl, thiophenyl, thiazolyl, morpholinyl n-methyl piperazinyl, benzothiophenyl, azetidinyl, pyrrolidinyl, piperidino or imidazolinyl) where when R₁₄ and R₁₅ together with the N form an heterocyclic ring, said ring may be substituted with 1, 2 or more of any of the substituents described herein, preferably piperazinyl, pyrrolidinyl, alkyl such as methyl, or hydroxy alkyl such as ethanyl. Examples of the heteroring formed by R₁₄ and R₁₅ together with the N include morpholinyl, which can be unsubstituted or substituted with methyl or dimethyl; piperazinyl which can be unsubstituted or substituted with 1, 2 or 3 substituents preferably methyl, oxy or ethanol; or piperadinyl which can be unsubstituted or substituted with 1, 2 or 3 substituents preferably pyrrolidinyl, amine, alkyl amine, methyl amine, dialkyl amine, dimethylamine or diethylamine;

A heteroaryl group usually is monocyclic, but may be bi- or tri-cyclic, and comprises 3-24 ring atoms, wherein at least one or more ring carbons are replaced by a heteroatom selected from O, N or S. The heteroaryl group is selected from, for example, pyridyl, indolyl, pyrimidyl, pyrazolyl, oxazolyl, thiophenyl, benzothiophenyl, 2H-pyrrolyl, pyrrolyl, imidazolyl, benzimidazolyl, pyrazolyl, indazolyl, purinyl, pyrazinyl, pyridazinyl, 4H-quinolizinyl, isoquinolyl, quinolyl, phthalazinyl, naphthyridinyl, quinoxalyl, quinazolinyl, quinolinyl, indolizinyl, 3H-indolyl, isoindolyl, isoxazolyl, thiazolyl, isothiazolyl, triazolyl, tetrazolyl, furazanyl and benzo[d]pyrazol.

In certain embodiments, the heteroaryl group is selected from the group consisting of pyridyl, indolyl, pyrimidyl, pyrazolyl, oxazolyl, thiophenyl or benzothiophenyl.

The heteroaryl group may be unsubstituted or substituted by one or more substituents selected from the group defined above as substituents for aryl, or by hydroxy, halogen, lower alkyl, such as methyl or lower alkoxy, such as methoxy or ethoxy.

Polyheterocycle as used herein refers to any nitrogen-substituted cycloalkyl, cycloalkenyl, aryl, cycloalkenylaryl, or cycloalkaryl, aromatic or non-aromatic, any of which may be further heterosubstituted. Examples include, e.g., C₃-C₆ cycloalkyl or partially saturated cycloalkyl, C₃-C₆ saturated or partially unsaturated heterocycloalkyl or heterocycloalkenyl (e.g., tetrahydro-pyridine), morpholine, C₃-C₆ heteroaryl, or a C₃-C₆ polyheteroaryl. The term also encompasses a nitrogen-substituted cycloalkyl, aryl or cycloalkaryl, aromatic or non-aromatic, which is fused or spiro to another cycloalkyl, aryl or cycloalkaryl, which may be further fused to another cycloalkyl, aryl or cycloalkaryl, and any of which may be further heterosubstituted. Examples include: decahydro-(iso)quinoline, tetrahydro-(iso)quinoline, piperazine, piperidine, indole, (iso)indole, benzyl, furan, or compounds of formula (Ia) through formula (If):

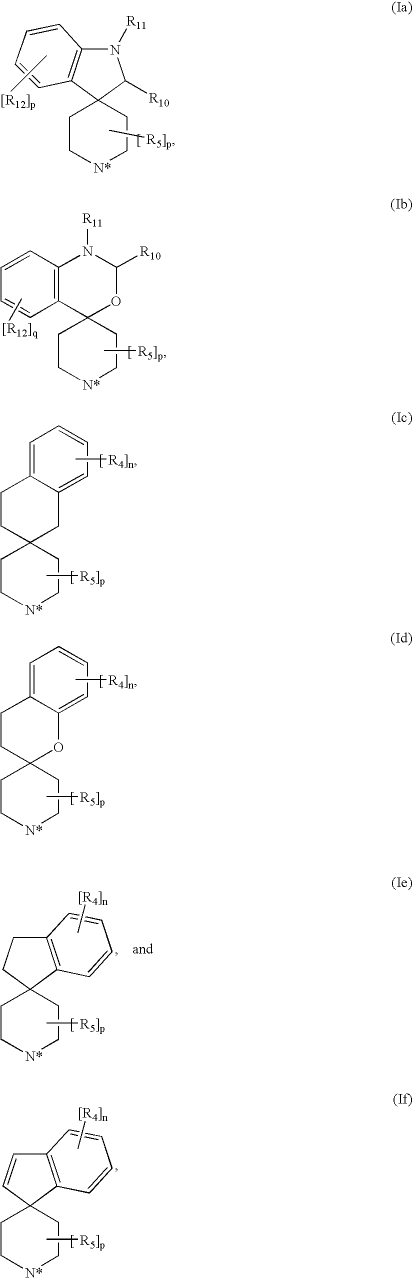

wherein N* designates the N to which is attached the peptide bond of formula I (i.e., is further substituted by —C(O)—CR₁R₂R₃), wherein R₁, R₂ and R₃ are as defined above.

Aliphatic as used herein refers to any non-aromatic carbon based residue. Examples of aliphatic residues include substituted or unsubstituted alkyl, cycloalkyl, alkenyl and alkynyl.

Alkyl includes lower alkyl, preferably alkyl with up to 7 carbon atoms, including, for example, from 1 to and including 5, and is linear or branched; in certain embodiments, lower alkyl is pentyl, such as n-pentyl, butyl, such as n-butyl, sec-butyl, isobutyl, tert-butyl, propyl, such as n-propyl or isopropyl, ethyl or methyl. In other embodiments, lower alkyl is methyl, propyl or tert-butyl.

A cycloalkyl group includes, for example, cyclopentyl, cyclohexyl or cycloheptyl, and may be unsubstituted or substituted by one or more substituents selected from the group defined above as substituents for aryl, lower alkyl such as methyl, lower alkoxy such as methoxy or ethoxy, or hydroxy.

Alkenyl and alkynyl preferably have up to 7 carbon atoms, including, for example, from 1 to and including 5, and can be linear or branched.

Alkyl, cycloalkyl, alkenyl and alkynyl can be substituted or unsubstituted, and when substituted may have with up to 3 substituents including other alkyl, cycloalkyl, alkenyl, alkynyl, any of the substituents defined above for aryl or any of the functional groups defined below.

Halo or halogen is preferably fluoro, chloro, bromo or iodo, most preferably fluoro, chloro or bromo.

The phrase, “connecting atom or group” as used herein includes alkyl (such as —CH₂—); oxy —O—; keto —CO—; thio —S—; sulfonyl —SO₂—; sulfoxides —SO—; amines —NH— or —NR—; carboxylic acid; alcohol; esters (—COO—); amides (—CONR—, —CONHR′—); sulfonamides (—SO₂NH—, —SO₂NR′—); sulfones (—SO₂—); sulfoxides (—SO—); amino-group; ureas (—NH—CO—NH—, —NR—CO—NH—, —NH—CO—NR—, —NR—CO—NR—); ethers (—O—); carbamates (—NH—CO—O—, —NR—CO—O—); and inverse amides sulfonamides and esters (—NH—CO—, —NR—CO—, —NH—SO₂—, —NR—SO₂—, —OOC—). R and R′ are the same or are different and may be H or are any aliphatic, aryl, heteroaryl, alkylaryl or heteroalkylaryl moiety as defined above.

The term “functional group” as used herein includes: carboxylic acid; hydroxyl; halogen; cyano (—CN); ethers (—OR); ketones (—CO—R); esters (—COOR); amides (—CONH2, —CONHR, —CONRR′); thioethers (—SR); sulfonamides (—SO₂NH₂, —SO₂NHR′), —SO₂NRR′); sulfones (—SO₂—R); sulfoxides (—SO—R); amines (—NHR, NR′R); ureas (—NH—CO—NH₂, —NH—CO—NHR); ethers (—O—R); halogens; carbamates (—NH—CO—OR); aldehyde-function (—CHO); then also inverse amides; and sulfonamides and esters (—NH—CO—R, —NH—SO₂—R, —OOC—R).

R and R′ are the same or are different and may be H or are any aliphatic, aryl, heteroaryl, alkylaryl or heteroalkylaryl moiety as defined above.

Where the plural form is used for compounds, salts, pharmaceutical preparations, diseases and the like, this is intended to mean also a single compound, salt, or the like.

Salts are, including for example, the pharmaceutically acceptable salts of compounds of the present invention.

Such salts are formed, for example, as acid addition salts, including for example with organic or inorganic acids, from compounds of the present invention with a basic nitrogen atom, including the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids such as glutamic acid or aspartic acid, maleic acid, hydroxymaleic acid, methylmaleic acid, cyclohexanecarboxylic acid, adamantanecarboxylic acid, benzoic acid, salicylic acid, phthalic acid, 4-aminosalicylic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, and other organic protonic acids, such as ascorbic acid.

In the presence of negatively charged radicals, such as carboxy or sulfo, salts may also be formed with bases, e.g., metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acidic group are present in the same molecule, a compound of the present invention may also form internal salts.

For isolation or purification purposes it is also possible to use salts that are not necessarily pharmaceutically acceptable, for example picrates or perchlorates. For therapeutic use, only pharmaceutically acceptable salts or free compounds are employed (where applicable in the form of pharmaceutical preparations).

In view of the close relationship between the compounds in free form and those in the form of their salts, including those salts that can be used as intermediates, for example in the purification or identification of the compounds, tautomers or tautomeric mixtures and their salts, any reference to the compounds herein before and hereinafter, is to be understood as referring also to the corresponding tautomers of these compounds, tautomeric mixtures of these compounds, or salts of any of these, as appropriate and expedient and if not mentioned otherwise.

Where “a compound . . . a tautomer thereof; or a salt thereof” or the like is mentioned, this means “a compound . . . , a tautomer thereof, or a salt of the compound or the tautomer”.

Any asymmetric carbon atom may be present in the (R)-, (S)- or (R,S)-configuration. Substituents at a ring at atoms with saturated bonds may, if possible, be present in cis-(=Z-) or trans (=E-) form. The compounds may thus be present as mixtures of isomers or as pure isomers, including enantiomer-pure diastereomers or pure enantiomers.

The present invention also relates to pro-drugs of a compound of the present invention that are converted in vivo to the compounds of the present invention as described herein. Any reference to a compound of the present invention is therefore to be understood as referring also to the corresponding pro-drugs of the compound of the present invention, as appropriate and expedient.

### Use in HDAC Dependent Diseases

The compounds of the present invention have valuable pharmacological properties and are useful in the treatment of diseases. In certain embodiments, useful compounds of the invention are useful in the treatment of HDAC dependent diseases, e.g., as drugs to treat proliferative diseases. Preferred compounds for the treatment of HDAC dependent diseases are non-hydroxamate, non-thio containing compounds of the invention.

The phrase “treatment of HDAC dependent diseases” refers to the prophylactic or therapeutic (including palliative and/or curing) treatment of these diseases, including for example, the diseases mentioned below.

The term “use” includes any one or more of the following embodiments of the invention, respectively: the use in the treatment of HDAC dependent diseases; the use for the manufacture of pharmaceutical compositions for use in the treatment of these diseases, e.g., in the manufacture of a medicament; methods of use of aminoalkyl derivatives in the treatment of these diseases; pharmaceutical preparations having aminoalkyl derivatives for the treatment of these diseases; and aminoalkyl derivatives for use in the treatment of these diseases; as appropriate and expedient, if not stated otherwise. In particular, diseases to be treated and are thus preferred for use of a compound of the present invention are selected from HDAC dependent (“dependent” meaning also “supported”, not only “solely dependent”) diseases, including those corresponding proliferative diseases, and those diseases that depend on HDAC1, HDAC2, HDAC3, HDAC4, HDAC5, HDAC6, HDAC7, HDAC8, HDAC9, HDAC10, HDAC11, or a HDAC complex (hereinafter “HDACs”) can therefore be used in the treatment of HDAC dependent diseases. The term “use” further includes embodiments of compositions herein which bind to an HDAC protein sufficiently to serve as tracers or labels, so that when coupled to a fluor or tag, or made radioactive, can be used as a research reagent or as a diagnostic or an imaging agent.

In certain embodiments, a compound of the present invention is used for treating HDAC-dependent diseases, i.e., a disease dependant upon an activity of at least one of the HDACs as described herein, and use of the compound of the present invention as an inhibitor of any one or more HDACs. It is envisioned that a use can be a treatment of inhibiting one or a subset of the group HDAC1, HDAC2, HDAC3, HDAC4, HDAC5, HDAC6, HDAC7, HDAC8, HDAC9, HDAC10, and HDAC11, and does not imply that all of these enzymes are inhibited to an equal extent by any of the compounds herein.

Also envisioned herein are demonstrations of the antitumor activity of compounds of the present invention in vivo.

Various embodiments of the compounds of the present invention have valuable pharmacological properties and are useful in the treatment of protein HDAC dependent diseases, e.g., as drugs to treat proliferative and hyperproliferative diseases, and other HDAC dependent diseases as listed throughout this disclosure. Various additional embodiments of the compounds of the present invention have valuable binding properties and are useful in diagnostic and labeling capacities and as imaging agents.

### Assays

The inhibition of HDAC activity may be measured as follows: The baculovirus donor vector pFB-GSTX3 is used to generate a recombinant baculovirus that expresses the HDAC polypeptide. Transfer vectors containing the HDAC coding region are transfected into the DH10Bac cell line (GIBCO) and plated on selective agar plates. Colonies without insertion of the fusion sequence into the viral genome (carried by the bacteria) are blue. Single, white colonies are picked and viral DNA (bacmid) are isolated from the bacteria by standard plasmid purification procedures. Sf9 cells or Sf21 (American Type Culture Collection) cells are then transfected in 25 cm² flasks with the viral DNA using Cellfectin reagent.

Determination of small scale protein expression in Sf9 cells: Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (at an approximately MOI of 5). After 3 days, the cells are scraped off the plate and centrifuged at 500 rpm for 5 minutes. Cell pellets from 10-20, 100 cm² plates, are re-suspended in 50 mL of ice-cold lysis buffer (25 mM tris-HCl, pH 7.5, 2 mM EDTA, 1% NP-40, 1 mM DTT, 1 mM P MSF). The cells are stirred on ice for 15 minutes and then centrifuged at 5,000 rpms for 20 minutes.

Purification of GST-tagged proteins: The centrifuged cell lysate is loaded onto a 2 mL glutathione-sepharose column (Pharmacia) and is washed 3× with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

Measure of enzyme activity: HDAC assays with purified GST-HDAC protein are carried out in a final volume of 30 μL containing 15 ng of GST-HDAC protein, 20 mM tris-HCl, pH 7.5, 1 mM MnCl2, 10 mM MgCl2, 1 mM DTT, 3 μg/mL poly(Glu,Tyr) 4:1, 1% DMSO, 2.0 μM ATP (γ-[³³P]-ATP 0.1 μCi). The activity is assayed in the presence or absence of inhibitors. The assay is carried out in 96-well plates at ambient temperature for 15 minutes under conditions described below and terminated by the addition of 20 μL of 125 mM EDTA. Subsequently, 40 μL of the reaction mixture are transferred onto IMMOBILON-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, a vacuum is connected and each well-rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of MICROSCINT™ (Packard). IC₅₀ values are calculated by linear regression analysis of the percentage inhibition of each compound in duplicate, at 4 concentrations (usually 0.01, 0.1, 1 and 10 μM).

IC₅₀ calculations

| | |
|---|---|
| Input: | 3 × 4 μL stopped assay on IMMOBILON |
| | membrane, not washed |
| background (3 wells): | assay with H₂O instead of enzyme |
| positive control (4 wells): | 3% DMSO instead of compound |
| bath control (1 well): | no reaction mix |

IC₅₀ values are calculated by logarithmic regression analysis of the percentage inhibition of each compound at 4 concentrations (usually 3- or 10-fold dilution series starting at 10 μM). In each experiment, the actual inhibition by reference compound is used for normalization of IC₅₀ values to the basis of an average value of the reference inhibitor:

Normalized IC₅₀=measured IC₅₀ average ref. IC₅₀/measured ref. IC₅₀

Example: Reference inhibitor in experiment 0.4 μM, average 0.3 μM
Test compound in experiment 1.0 μM, normalization: 0.3/0.4=0.75 μM

For example, known HDAC inhibitors or a synthetic derivative thereof may be used as reference compounds.

Using this protocol, the compounds of the invention are found to show IC₅₀ values for HDAC inhibition in the range from 0.005-100 μM, or 0.002-50 μM, including, for example, the range from 0.001-2 μM or less.

### Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively tinder physiological conditions (e.g., by enzymatic cleavage).

Salts of compounds of the present invention having at least one salt-forming group may be prepared in a manner known per se. For example, salts of compounds of the present invention having acid groups may be formed, for example, by treating the compounds with metal compounds, such as alkali metal salts of suitable organic carboxylic acids, e.g., the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g., by treating the compounds with an acid or a suitable anion exchange reagent. Internal salts of compounds of the present invention containing acid and basic salt-forming groups, e.g., a free carboxy group and a free amino group, may be formed, e.g., by the neutralisation of salts, such as acid addition salts, to the isoelectric point, e.g., with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

### General Process Conditions

The following applies in general to all processes mentioned throughout this disclosure.

The process steps to synthesize the compounds of the invention can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

### Proliferative Diseases

As discussed above, the compounds of the present invention are useful for treating proliferative diseases. A proliferative disease includes, for example, a tumor disease (or cancer) and/or any metastases). The inventive compounds are useful for treating a tumor which is, for example, a breast cancer, genitourinary cancer, lung cancer, gastrointestinal cancer, esophageal cancer, epidermoid cancer, melanoma, ovarian cancer, pancreas cancer, neuroblastoma, head and/or neck cancer or bladder cancer, or in a broader sense renal, brain or gastric cancer; including (i) a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (including a hormone-refractory prostate tumor); or (ii) a proliferative disease that is refractory to the treatment with other chemotherapeutics; or (iii) a tumor that is refractory to treatment with other chemotherapeutics due to multidrug resistance.

TABLE 1
HDAC 1-11 genes with O.M.I.M accession
number and chromosomal locus

| Histone deacetylase | OMIM accession number | Chromosomal locus |
|---|---|---|
| HDAC1 | *601241 | 1p34.1 |
| HDAC2 | *605164 | 6q21 |
| HDAC3 | *605166 | 5q31 |
| HDAC4 | *605314 | 2q37.2 |
| HDAC5 | *605315 | Chr.17 |
| HDAC6 | *300272 | Xp11.23 |
| HDAC7A | *606542 | Chr.12 |
| HDAC8 | *300629 | Xq13 |
| HDAC9 | *606543 | 7p21-p15 |
| HDAC10 | *608544 | 22q13.31-q13.33 |
| HDAC11 | *607226 | 3p25.2 |

An HDAC dependent disease is any pathology related to expression of one or more of the genes encoding one of the HDAC proteins or HDAC-associated proteins, or an activity of such as protein, in that inhibition of the protein results in remediation of the pathology. The HDAC genes and proteins are as described in the Online Mendelian Inheritance in Man (O.M.I.M). Inhibition of an HDAC protein provides remediation of an HDAC dependent disease. Table 1 lists the HDAC proteins and the locus of each on the human genome. Table 2 shows HDAC 1-11 GenBank accession numbers for representative amino acid sequences in at least three organismal species when available.

TABLE 2
GenBank accession numbers for exemplary amino
acid sequences of HDAC1-11 proteins

| | GenBank amino | |
|---|---|---|
| Histone | acid sequence | |
| deacetylase protein | accession number | Source |
| HDAC1 | O60341 | Human |
| | NP_033214 | Mouse |
| | NP_571138 | Zebra fish |
| HDAC2 | NP_032255 | Human |
| | P70288 | Mouse |
| HDAC3 | NP_006302 | Human |
| | NP_034541 | Mouse |
| | NP_957284 | Zebra fish |
| HDAC4 | NP_005648 | Human |
| | NP_989644 | Chicken |
| | AAX52490 | Fruit fly |
| HDAC5 | NP_001015033 | Human |
| | AAS77826 | Porcine |
| | NP_034542 | Mouse |
| HDAC6 | Q9C2B2 | Human |
| | NP_034543 | Mouse |
| | AAH43813 | African clawed frog |
| HDAC7 | NP_057680 | Human |
| | AAK11188 | Norway rat |
| | Q8C2B3 | Mouse |
| HDAC8 | Q9BY41 | Human |
| | Q8VH37 | Mouse |
| | AAH55541 | Zebra fish |
| HDAC9 | Q9UKV0 | Human |
| | NP_07738 | Mouse |
| | NP_957110 | Zebra fish |
| HDAC10 | Q969S8 | Human |
| | Q569C4 | Norway rat |
| | NP_954668 | Mouse |
| HDAC11 | Q96DB2 | Human |
| | Q91WA3 | Mouse |

In certain embodiments, the proliferative disease may furthermore be a hyperproliferative condition such as leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

Where a tumor, a tumor disease, a carcinoma or a cancer are mentioned, also metastasis in the original organ or tissue and/or in any other location are implied alternatively or in addition, whatever the location of the tumor and/or metastasis.

The compounds described herein are selectively toxic or more toxic to rapidly proliferating cells than to normal cells, including, for example, human cancer cells, e.g., cancerous tumors, the compounds have significant antiproliferative effects and promotes differentiation, e.g., cell cycle arrest and apoptosis. In addition, the compounds induce p21, cyclin-CDK interacting protein, which induces either apoptosis or G1 arrest in a variety of cell lines.

The following examples are intended to illustrate the invention and are not to be construed as being limitations thereto.

In the following embodiments, general expression can be replaced by the corresponding more specific definitions provided above and below.

In certain embodiments, the use of compounds of the present invention, tautomers thereof or pharmaceutically acceptable salts thereof, where the HDAC dependent disease to be treated is a proliferative disease depending on any one or more of the following HDACs, including, for example, HDAC1, HDAC2, HDAC6 and HDAC8.

In other embodiments, the HDAC dependant disease may be a proliferative disease including a hyperproliferative condition, such as leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

In other embodiments, the invention provides a method of treating a HDAC dependent disease comprising administering a compound of the present invention, where the disease to be treated is a proliferative disease, including, for example, a benign or malignant tumor, a carcinoma of the brain, kidney, liver, adrenal gland, bladder, breast, stomach (including gastric tumors), esophagus, ovaries, colon, rectum, prostate, pancreas, lung (including SCLC), vagina, thyroid, sarcoma, glioblastomas, multiple myeloma or gastrointestinal cancer, especially colon carcinoma or colorectal adenoma, or a tumor of the neck and head, an epidermal hyperproliferation, including psoriasis, prostate hyperplasia, a neoplasia, including those of epithelial character, including mammary carcinoma, or a leukemia. Also included is a method for the treatment of atherosclerosis, thrombosis, psoriasis, scleroderma and fibrosis.

Compounds of the present invention are able to bring about the regression of tumors and to prevent the formation of tumor metastases (including micrometastases) and the growth of—metastases (including micrometastases). In addition they can be used in epidermal hyperproliferation (e.g., psoriasis), in prostate hyperplasia, and in the treatment of neoplasias, including that of epithelial character, for example mammary carcinoma. It is also possible to use the compounds of the present invention in the treatment of diseases of the immune system insofar as one or more individual HDAC protein species or associated proteins are involved. Furthermore, the compounds of the present invention can be used also in the treatment of diseases of the central or peripheral nervous system where signal transmission by at least one HDAC protein is involved.

HDAC inhibitors are also appropriate for the therapy of diseases related to transcriptional regulation of proteins involved in signal transduction, such as VEGF receptor tyrosine kinase overexpression. Among these diseases are retinopathies, age-related macula degeneration, psoriasis, haemangioblastoma, haemangioma, arteriosclerosis, muscle wasting conditions such as muscular dystrophies, cachexia, Huntington's syndrome, inflammatory diseases such as rheumatoid or rheumatic inflammatory diseases, including arthritis and arthritic conditions, such as osteoarthritis and rheumatoid arthritis, or other chronic inflammatory disorders such as chronic asthma, arterial or post-transplantational atherosclerosis, endometriosis, and especially neoplastic diseases, for example so-called solid tumors (including cancers of the gastrointestinal tract, the pancreas, breast, stomach, cervix, bladder, kidney, prostate, esophagus, ovaries, endometrium, lung, brain, melanoma, Kaposi's sarcoma, squamous cell carcinoma of head and neck, malignant pleural mesotherioma, lymphoma or multiple myeloma) and liquid tumors (e.g., leukemias).

HDAC proteins share a set of nine consensus sequences. HDAC proteins are classified into two classes based on amino acid sequence: class I proteins such as HDAC1, HDAC2 and HDAC3 have substantial homology to yeast Rpd3; class II such as HDAC4 and HDAC6 show homology to yeast Hda1. Various facts indicate an association of these proteins with the HDAC dependent diseases.

HDAC1 is a protein having 482 amino acids, and is highly conserved in nature, having 60% identity to a yeast transcription factor. It is found at various levels in all tissues, and is involved in transcriptional regulation and cell cycle progression, particularly G1 checkpoint control. HDAC1 interacts physically with and cooperates with RB1, the retinoblastoma tumor suppressor protein that inhibits cell proliferation, and with nuclear transcription factor NFκB.

HDAC2 is also known as YY1-associated factor (YAF1), as it associates with mammalian zinc finger transcription factor YY1. The locus that encodes this protein on the human genome is 6q21, a region of the genome implicated in childhood acute lymphocytic leukemia (ALL) and ulnar ray limb defect. Further, HDAC2 interacts with and is physically associated with BRCA1 in a complex that includes also HDAC1. The common core of this complex functions to repress genes to a silent condition. A different complex is formed during S phase, and histone is deacetylated into heterochromatin following replication.

HDAC3 is known to be expressed in all human tissues and tumor cell lines. Transfection of a human myeloid leukemia line resulted in accumulation of cells at the G2/M boundary phase with aberrant nuclear morphology and increased cell size. The catalytic domain of HDAC4 interacts with HDAC3.

HDAC4 deacetylase activity acts on all four core histone proteins, and is expressed in prehypertrophic chondrocytes and regulates chondrocyte hypertrophy, endochondral bone formation and skeletogenesis. HDAC4-null mice display premature ossification. With MIR and CABIN1, HDAC4 constitutes a family of calcium-sensitive transcriptions repressors of MEF-2 (myocyte enhancer factor-2).

HDAC5 is expressed in all tissues tested, with lower expression in spleen and pancreas. The 1,123 amino acid sequence of HDAC5 is 51% identical to HDAC4. Five of 29 colon cancer patients tested serologically positive for antibody to HDAC5. MEF-2 protein interacts with HDAC4 and HDAC5.

HDAC6 is a tubulin deacetylase and is localized exclusively in cytoplasm. This enzyme has potent deacetylase activity for assembled microtubules and therapeutic intervention into its expression or activity can be associated with a variety of conditions affecting muscle integrity and muscle wasting, such as Huntington's disease and cachexia.

HDAC7A transcript is found predominantly in heart and lung tissues, and to a lesser extent in skeleton muscle. The protein co-localizes with HDAC5 in subnuclear regions.

HDAC8 is a 377 amino acid protein which while possessing the typical nine conserved HDAC blocks of consensus sequence, has sequences at each of the amino and carboxy termini that are distinct from those of other HDAC proteins. It is expressed most strongly in brain. Knockdown of expression by RNAi inhibits growth of human lung, colon, and cervical cancer cell lines. The map position of the encoding gene at Xq13 is located near XIST which is involved in initiation of X chromosome inactivation, and near breakpoints associated with preleukemia conditions. Further, therapeutic intervention into its expression or activity can be associated with a variety of conditions affecting inflammatory diseases such as various arthritic conditions, e.g., rheumatoid arthritis.

HDAC9 is known also as 7B, MITR, and KLAA0744. It is expressed most actively in brain, and to a lesser extent in heart and smooth muscle, and very little in other tissues. This protein interacts with HDAC1 and is a repressor of transcription. A longer isoform contains 1,011 amino acids and a shorter form, known as 9a, contains 879 amino acids, lacking 132 residues at the C-terminus, predominates in lung, liver and skeletal muscle.

HDAC10 is found in two splice variants of 669 and 649 amino acids. The protein represses transcription from a thymidine kinase promoter and interacts with HDAC3.

HDAC11 is a 347 amino acid protein that is expressed most highly in brain, heart, skeletal muscle, kidney and testis. It partitions with nuclear extracts.

Angiogenesis is regarded as an absolute prerequisite for those tumors which grow beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size.

Three principal mechanisms play an important part in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells which normally line the vessels.

The present invention can also be used to prevent or treat diseases that are triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

### Pharmaceutical Compositions

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

The invention relates also to pharmaceutical compositions comprising a compound of the present invention, to their use in the therapeutic (in a broader aspect of the invention also prophylactic) treatment or a method of treatment of a HDAC dependent disease, including, for example, the diseases mentioned above, to the compounds for the use and to the preparation of pharmaceutical preparations, for the uses.

The present invention also relates to pro-drugs of a compound of the present invention that convert in vivo to the compound of the present invention as such. Any reference to a compound of the present invention is therefore to be understood as referring also to the corresponding pro-drugs of the compound of the present invention, as appropriate and expedient.

The pharmacologically acceptable compounds of the present invention may be used, for example, for the preparation of pharmaceutical compositions that comprise an effective amount of a compound of the present invention, or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, including, for example, a human (or to cells or cell lines derived from a warm-blooded animal, including for example, a human cell, e.g., lymphocytes), for the treatment or, in another aspect of the invention, prevention of (also referred to as prophylaxis against) a disease that responds to inhibition of HDAC activity, comprising an amount of a compound of the present invention or a pharmaceutically acceptable salt thereof, which is effective for this inhibition, including the inhibition of activity of an HDAC or inhibition of an HDAC protein interacting with another transcriptional effector protein, together with at least one pharmaceutically acceptable carrier.

The pharmaceutical compositions according to the invention are those for enteral, such as nasal, rectal or oral, or parenteral, such as intramuscular or intravenous, administration to warm-blooded animals (including, for example, a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The dose of a compound of the present invention or a pharmaceutically acceptable salt thereof to be administered to warm-blooded animals, for example humans of approximately 70 kg body weight, is for example, from approximately 3 mg to approximately 10 g, from approximately 10 mg to approximately 1.5 g, from about 100 mg to about 1000 mg/person/day, divided into 1-3 single doses which may, for example, be of the same size. Usually, children receive half of the adult dose.

The pharmaceutical compositions have from approximately, for example, 1% to approximately 95%, or from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are used, it being possible, for example in the case of lyophilized compositions that have the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The solutions or suspensions may have viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned, for example, liquid fatty acid esters that contain as the acid component a long-chained fatty acid having from 8-22, or from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hills AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are for example, fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose; binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

### Combinations

A compound of the present invention may also be used to advantage in combination with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MM inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMODAL®); and leucovorin.

The phrase, “aromatase inhibitor” as used herein relates to a compound which inhibits the estrogen production, i.e., the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g., under the trademark AROMASIN. Formestane can be administered, e.g., in the form as it is marketed, e.g., under the trademark LENTARON. Fadrozole can be administered, e.g., in the form as it is marketed, e.g., under the trademark AFEMA. Anastrozole can be administered, e.g., in the form as it is marketed, e.g., under the trademark ARIMIDEX. Letrozole can be administered, e.g., in the form as it is marketed, e.g., under the trademark FEMARA or FEMAR. Aminoglutethimide can be administered, e.g., in the form as it is marketed, e.g., under the trademark ORIMETEN. A combination of the invention comprising a chemotherapeutic agent which is an aromatase inhibitor is particularly useful for the treatment of hormone receptor positive tumors, e.g., breast tumors.

The term “antiestrogen” as used herein relates to a compound that antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g., under the trademark NOLVADEX. Raloxifene hydrochloride can be administered, e.g., in the form as it is marketed, e.g., under the trademark EVISTA. Fulvestrant can be formulated as disclosed in U.S. Pat. No. 4,659,516 or it can be administered, e.g., in the form as it is marketed, e.g., under the trademark FASLODEX. A combination of the invention comprising a chemotherapeutic agent which is an antiestrogen is particularly useful for the treatment of estrogen receptor positive tumors, e.g., breast tumors.

The term “anti-androgen” as used herein relates to any substance which is capable of inhibiting the biological effects of androgenic hormones and includes, but is not limited to, bicalutamide (CASODEX), which can be formulated, e.g., as disclosed in U.S. Pat. No. 4,636,505.

The phrase, “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g., under the trademark ZOLADEX. Abarelix can be formulated, e.g., as disclosed in U.S. Pat. No. 5,843,901.

The phrase, “topoisomerase I inhibitor” as used herein includes, but is not limited to topotecan, gimatecan, irinotecan, camptothecan and its analogues, 9-nitrocamptothecin and the macromolecular camptothecin conjugate PNU-166148 (compound A1 in WO99/17804). Irinotecan can be administered, e.g., in the form as it is marketed, e.g., under the trademark CAMPTOSAR. Topotecan can be administered, e.g., in the form as it is marketed, e.g., under the trademark HYCAMTIN.

The phrase, “topoisomerase II inhibitor” as used herein includes, but is not limited to the anthracyclines such as doxorubicin (including liposomal formulation, e.g., CAELYX), daunorubicin, epirubicin, idarubicin and nemorubicin, the anthraquinones mitoxantrone and losoxantrone, and the podophyllotoxins etoposide and teniposide. Etoposide can be administered, e.g., in the form as it is marketed, e.g., under the trademark ETOPOPHOS. Teniposide can be administered, e.g., in the form as it is marketed, e.g., under the trademark VM 26-BRISTOL. Doxorubicin can be administered, e.g., in the form as it is marketed, e.g., under the trademark ADRIBLASTIN or ADRIAMYCIN. Epirubicin can be administered, e.g., in the form as it is marketed, e.g., under the trademark FARMORUBICIN. Idarubicin can be administered, e.g., in the form as it is marketed, e.g., under the trademark ZAVEDOS. Mitoxantrone can be administered, e.g., in the form as it is marketed, e.g., under the trademark NOVANTRON.

The phrase, “microtubule active agent” relates to microtubule stabilizing, microtubule destabilizing agents and microtublin polymerization inhibitors including, but not limited to taxanes, e.g., paclitaxel and docetaxel, vinca alkaloids, e.g., vinblastine, including vinblastine sulfate, vincristine including vincristine sulfate, and vinorelbine, discodermolides, cochicine and epothilones and derivatives thereof, e.g., epothilone B or D or derivatives thereof. Paclitaxel may be administered e.g., in the form as it is marketed, e.g., TAXOL. Docetaxel can be administered, e.g., in the form as it is marketed, e.g., under the trademark TAXOTERE. Vinblastine sulfate can be administered, e.g., in the form as it is marketed, e.g., under the trademark VINBLASTIN R.P. Vincristine sulfate can be administered, e.g., in the form as it is marketed, e.g., under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, —WO 99/43653, WO 98/22461 and WO 00/31247. Included are Epothilone A and/or B.

The phrase, “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g., under the trademark CYCLOSTIN. Ifosfamide can be administered, e.g., in the form as it is marketed, e.g., under the trademark HOLOXAN.

The phrase, “histone deacetylase inhibitors” or “HDAC inhibitors” relates to compounds which inhibit at least one example of the class of enzymes known as a histone deacetylase, as described herein, and which compounds generally possess antiproliferative activity. Previously disclosed HDAC inhibitors include compounds disclosed in, e.g., WO 02/22577, including N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further includes Suberoylanilide hydroxamic acid (SAHA). Other publicly disclosed HDAC inhibitors include butyric acid and its derivatives, including sodium phenylbutyrate, thalidomide, trichostatin A and trapoxin.

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate, and folic acid antagonists such as pemetrexed. Capecitabine can be administered, e.g., in the form as it is marketed, e.g., under the trademark XELODA. Gemcitabine can be administered, e.g., in the form as it is marketed, e.g., under the trademark GEMZAR. Also included is the monoclonal antibody trastuzumab which can be ad-ministered, e.g., in the form as it is marketed, e.g., under the trademark HERCEPTIN.

The phrase, “platin compound” as used herein includes, but is not limited to, carboplatin, cis-platin, cisplatinum and oxaliplatin. Carboplatin can be administered, e.g., in the form as it is marketed, e.g., under the trademark CARBOPLAT. Oxaliplatin can be administered, e.g., in the form as it is marketed, e.g., under the trademark ELOXATIN.

The phrase, “compounds targeting/decreasing a HDAC activity; or a histone deacetylase activity; or further anti-angiogenic compounds” as used herein includes, but is not limited to: HDAC1-11 inhibitors, e.g.: HDAC2, HDAC3 AND HDAC8 inhibitors.

The following list of proteins involved in signal transduction illustrates far reaching effects of modulating transcription by inhibiting HDAC activity:

i) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g., a N-phenyl-2-pyrimidine-amine derivative, e.g., imatinib, SU101, SU6668, and GFB-111;

ii) compounds targeting, decreasing or inhibiting the activity of the fibroblast growth factor-receptors (FGFR);

iii) compounds targeting, decreasing or inhibiting the activity of the insulin-like growth factor receptor I (IGF-IR), such as compounds which target, decrease or inhibit the activity of IGF-IR, especially compounds which inhibit the IGF-IR receptor, such as those compounds disclosed in WO 02/092599; and/or

iv) compounds targeting, decreasing or inhibiting the activity of the c-Met receptor.

Tumor cell damaging approaches refer to approaches such as ionizing radiation. The phrase, “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See, e.g., Hellman, Principles of Radiation Therapy, Cancer, in Principles and Practice of Oncology, Devita et al., Eds., 4th Edition, Vol. 1, pp. 248-275 (1993).

The phrase, “EDG binders” as used herein refers a class of immunosuppressants that modulates lymphocyte recirculation, such as FTY720.

CERTICAN (everolimus, RAD) an investigational novel proliferation signal inhibitor that prevents proliferation of T-cells and vascular smooth muscle cells.

The phrase, “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The phrase, “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g., 1-(4-chloroanilino)-4-(4-pyridylmethyl)phthalazine or a pharmaceutically acceptable salt thereof, e.g., the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g., rhuMAb and RHUFab, VEGF aptamer e.g., Macugon; FLT-4 inhibitors, FLT-3 inhibitors, VEGFR-2 IgG1 antibody, Angiozyme (RPI 4610) and Avastan.

Photodynamic therapy as used herein refers to therapy that uses certain chemicals known as photosensitizing agents to treat or prevent cancers. Examples of photodynamic therapy include treatment with agents, such as e.g., VISUDYNE and porfimer sodium.

The phrase, “angiostatic steroids” as used herein refers to agents which block or inhibit angiogenesis, such as, e.g., anecortave, triamcinolone. hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to agents, such as e.g., fluocinolone, dexamethasone.

Other chemotherapeutic agents include, but are not limited to, plant alkaloids, hormonal agents and antagonists; biological response modifiers, preferably lymphokines or interferons; antisense oligonucleotides or oligonucleotide derivatives; or miscellaneous agents or agents with other or unknown mechanism of action.

The structure of the active agents identified by code numbers, generic or trade names may be taken from the actual edition of the standard compendium “The Merck Index” or from databases, e.g., Patents International (e.g., IMS World Publications).

The above-mentioned compounds, which can be used in combination with a compound of the present invention, can be prepared and administered as described in the art such as in the documents cited above.

A compound of the present invention may also be used to advantage in combination with known therapeutic processes, e.g., the administration of hormones or especially radiation.

A compound of the present invention may in also be used as a radiosensitizer, including, for example, the treatment of tumors which exhibit poor sensitivity to radiotherapy.

By the term “combination”, is meant either a fixed combination in one dosage unit form, or a kit of parts for the combined administration where a compound of the present invention and a combination partner may be administered independently at the same time or separately within time intervals that especially allow that the combination partners show a cooperative, e.g., synergistic, effect, or any combination thereof.

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated herein by reference.

### EXAMPLES

### Example 1

### General Methods

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the aminoalkyl compounds of the present invention can be produced by organic synthesis methods known to one of ordinary skill in the art as shown in the following Examples.

### General Methods for Organic Synthesis of Amides

A comprehensive overview of methods available to synthesize amides is given in Houben-Weyl 4th Ed. 1952, Methods of Organic Synthesis, Thieme, Volume 21. One synthesis protocol provided by this reference involves coupling an acid with an amine to produce amide compounds, which are compounds of the present invention. As an example, the commercially available (R)-2-(tert-butoxycarbonyl)-3-phenylpropanoic acid (1) can be reacted with the commercially available isoindoline (2) to form amide 3 in the presents of a dehydrating agent (e.g., 2-(2-pyridon-1-yl)-1,1,3,3-tetramethyluronium tetrafluoroborate, TPTU) and a base (e.g., N-methylmorpholine, NMM) in an appropriate solvent (e.g., dichloromethane, DCM). The amide 3 can be deprotected to the amino-amide 4, a final compound of the present invention, by treatment with an organic acid (e.g., trifluoroacetic, TFA) or an inorganic acid (e.g., hydrochloric acid, HCl) in DCM or methanol (MeOH).

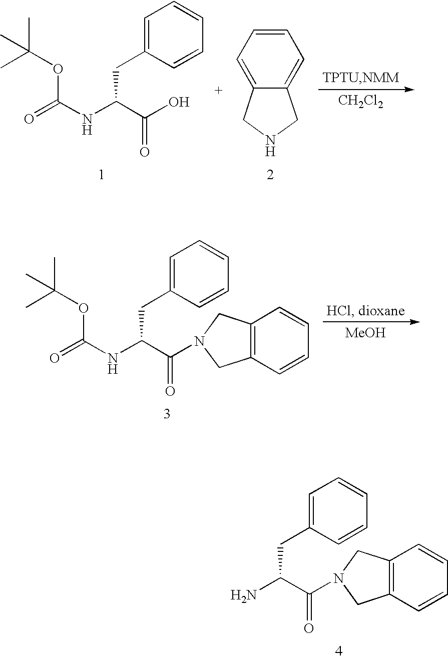

Alternatively the amino compounds of the present invention can be synthesized applying Weinreb-type chemistry (Tetrahedron Letters 2000, 41(8): 1141). As an example, the commercially available isoindoline 2 can be coupled to a commercially available N-protected amino ester (e.g., N-(diphenylmethylene)glycine ethyl ester) 5 to form the amide 6 by first treating the amine 2 with an organo aluminum species such as trimethyl aluminum and adding the resulting organometallic intermediate to the protected amino ester 5 in an appropriate solvent such as DCM. Treatment of 6 with benzylbromide in the presence of a phase transfer catalyst (e.g., tetrabutylammonium bromide, TBAB) and a suitable base (e.g., potassium hydroxide, KOH) and a solvent such as DCM yields 7, (Journal of the American Chemical Society, 1989, 111(6):2353), which upon treatment with an acid (e.g., HCl) in a suitable solvent such as DCM produces 8, a compound of the present invention.

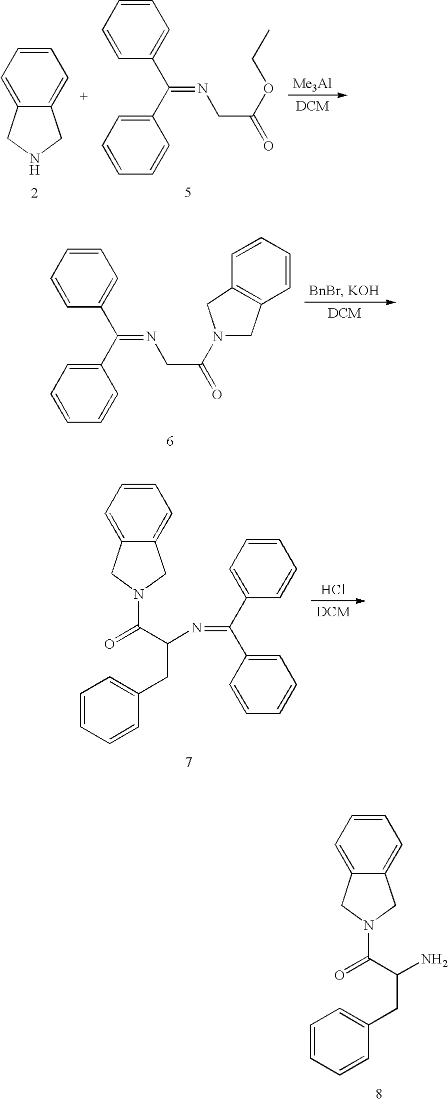

### General Methods for Organic Synthesis of Acids

Acids used to produce the aminoalkyl compounds of the present invention are either commercially available, can be synthesized by methods known in the literature to one of ordinary skill in the art, or can be synthesized utilizing organic synthesis methods known to one of ordinary skill in the art. For example non-commercial amino acids can be prepared by either chiral phase transfer alkylation of an imino glycine ester, similar to the protocol shown above, (Journal of the American Chemical Society 1989, 111(6):2353) or by an olefination of the a phosphono glycine ester and subsequent asymmetric reduction as shown below (Tetrahedron 2002, 58(36):7365).

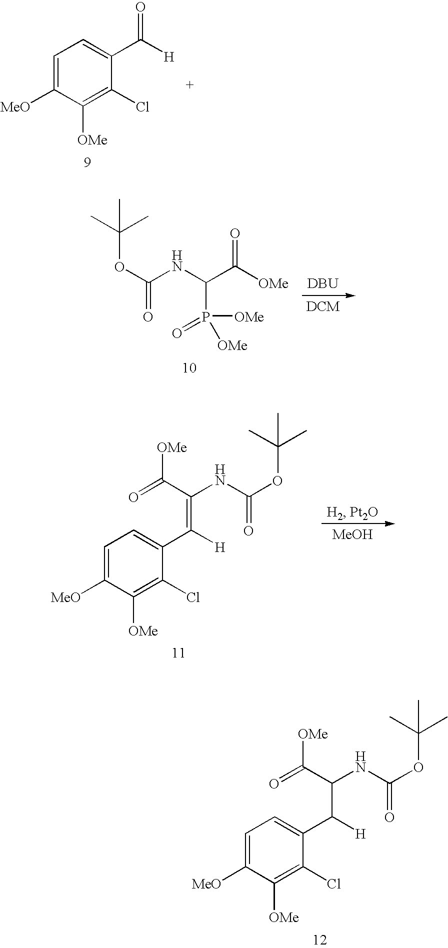

In this exemplary protocol, amino acid 12 can be synthesized by a route involving olefination of an aldehyde 9 with a phoshpono ester 10 in the presence of a strong base such as DBU in an appropriate solvent such as DCM. The resulting dehydro amino acid 11 can be reduced in a hydrogen atmosphere in the presence of a transition metal catalyst such as platinum oxide (Pt₂O) affording the protected alpha amino acid 12.

### General Analytical Conditions

Detection can be made by UV light (254 nm). HPLC is performed on an Agilent HP 1100 using a Nucleosil 100-3 C₁₈ HD 125×4.0 mm column [1 mL/min.; 20-100% NeCN/0.1% TFA in 7 minutes); SpectraSystem SP8800/UV2000 using a Nucleosil 100-5 C₁₈ AB 250×4.6 mm column (2 mL/min.; 2-100% MeCN/0.1% TFA in 10 minutes); using a Chromalith Speed ROD RP18 50-4.6 mm column (Merck), (2 mL/min.; 2-100% MeCN/0.1% TFA in 2 minutes); or a C8 2.1-50 mm 3 μm column (Waters) (2 mL/min.; 5-95% MeCN/0.1% TFA in 2 minutes).

NMR measurements are performed on a Varian Gemini 400 or a Bruker DRX 500 spectrometer using tetraethylsilane as internal standard. Chemical shifts are expressed in ppm downfield from tetraethylsilane and coupling constants (J) are expressed in Hertz (Hz). Electrospray mass spectra are obtained with a Fisons Instruments VG Platform II. Melting points are measured with a Büchi 510 melting point apparatus. Commercially available solvents and chemicals are used for syntheses.

### Example 2

### Synthesis of Core Formula III

Core Formula III is synthesized from amines that are either commercially available, can be synthesized by methods known in the literature to one of ordinary skill in the art, or can be synthesized utilizing organic synthesis methods known to one of ordinary skill in the art. One of ordinary skill in the art will know that further reactions of the core intermediate in series or in parellel will result in product aminoalkyl compounds of the present invention, as shown in further Examples 7-26.

For example, amines used to synthesize compounds of scaffold III can be prepared using Suzuki-type coupling methodology and by employing Pd-metal modified with a variety of phosphines (Journal of the American Chemical Society 1999, 121:9550; Synthesis 2004, 15:2419).

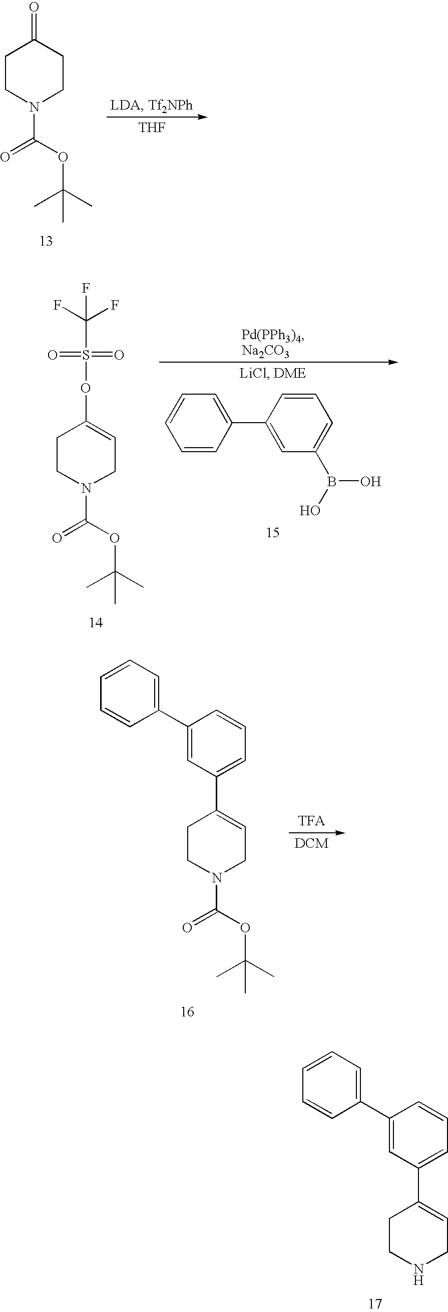

As an example, commercially available 4-oxo-piperidine-1-carboxylic acid tert-butyl ester (13) can be transformed into triflate 14 by treatment with a triflating agent (e.g., N-phenyltrifluoromethanesulfonamide, Tf₂NPh) and a base (e.g., lithiumdiisopropyl amine, LDA) in an appropriate solvent (e.g., tetrahydrofuran, THF) and under low temperature (e.g., from −78° C. to 0° C.). Triflate 14 can be transformed into piperidine 16 via Suzuki protocol (Synthesis 2004, 15:2419; Journal of the American Chemical Society 1999, 121:9550), using a palladium catalyst (e.g., Pd(PPh₃)₄), an appropriate biphasic solvent such as dimethoxyethane (DME) and water and a base (e.g., sodium carbonate, Na₂CO₃), an appropriate additive (e.g., lithium chloride, LiCl) and a commercially available boronic acid (e.g., biphenyl-3-boronic acid 15) under elevated temperatures (e.g., from 30° C.-90° C.). Piperidine intermediate 16 is transformed to a piperidine 17 in a free base form or as the hydrochloride salt by treatment with an appropriate organic acid (e.g., TFA) and by subsequent treatment with inorganic acid (e.g., hydrochloric acid, HCl).

### Example 3

### Synthesis of Core Formula IV

Core Formula IV is synthesized from amines that are either commercially available, can be synthesized by methods known in the literature to one of ordinary skill in the art, or can be synthesized utilizing organic synthesis methods known to one of ordinary skill in the art. One of ordinary skill in the art will know that further reactions of the core intermediate in series or in parallel will result in product aminoalkyl compounds of the present invention, as shown in further Examples 7-26.

As an example, amines used to synthesize compounds of scaffold IV can be prepared by reduction and deprotection of the piperidines provided in Example 2 above. For example, piperidine 16 can be deprotected using an organic acid (e.g., trifluoroacetic acid, TFA) in an appropriate solvent (e.g., dichloromethane DCM) and hydrogenated using a palladium catalyst and hydrogen gas (e.g., 10% palladium on charcoal/50 psi of hydrogen gas) in appropriate solvent (e.g., methanol, MeOH) to produce reduced piperidine intermediate.

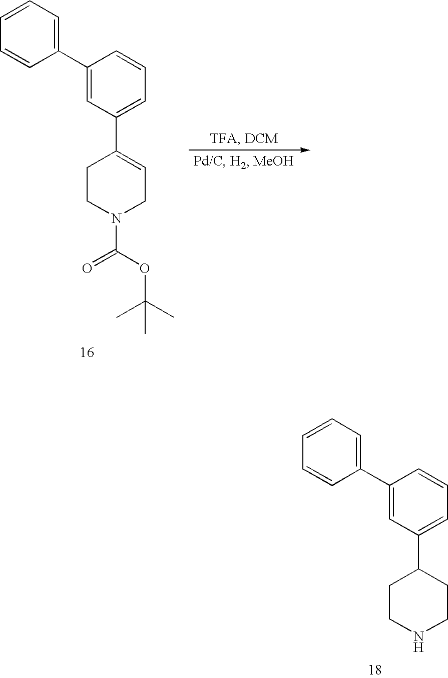

### Example 4

### Synthesis of Core Formula V

Core Formula V is synthesized from piperazines that are either commercially available, can be synthesized by methods known in the literature to one of ordinary skill in the art, or can be synthesized utilizing organic synthesis methods known to one of ordinary skill in the art. One of ordinary skill in the art will know that further reactions of the core intermediate in series or in parallel will result in product aminoalkyl compounds of the present invention, as further shown in Examples 7-26.

For example, piperazines used to synthesize compounds of scaffold V can be prepared by derivatizing a mono-protected piperazine. The derivatization can be done by a number of known methods, including but not limited to, the one shown below.

Commercially available 1-Boc-piperazine (19) can be coupled to an acyl chloride (e.g., benzoylchloride, 20) in appropriate solvent (e.g., DCM) using a base (e.g., triethyl amine, Et₃N) to produce amide 21. Amide 21 is then transformed to piperazine 22, which is core Formula V, by treatment with an organic acid (e.g., TFA).

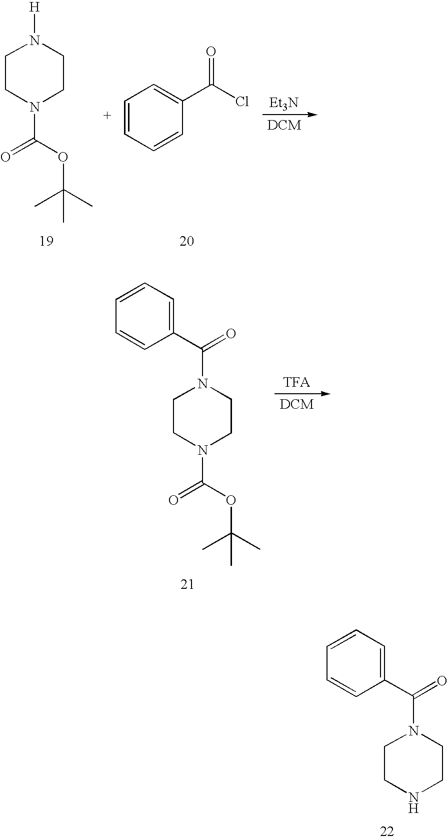

### Example 5

### Synthesis of Core SubFormulae VIa-VIj

### Synthesis of Core SubFormulae VIa-VIf

Core Formulae VIa-VIf are synthesized from spiro-piperidines that are either commercially available, can be synthesized by methods known in the literature to one of ordinary skill in the art (e.g., Journal of Medicinal Chemistry 1992, 35(21):3919), or can be synthesized utilizing organic synthesis methods known to one of ordinary skill in the art (Houben-Weyl, Methods of Organic Synthesis, Thieme, Volume 21). One of ordinary skill in the art will know that further reactions of the core intermediate in series or in parallel will result in product aminoalkyl compounds of the present invention, as further shown in Examples 7-26.

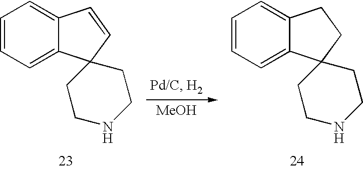

For example the spiro-piperidine 23 can be hydrogenated over a suitable catalyst (e.g., Pd/C) at ambient temperature under a hydrogen atmosphere in an appropriate solvent like methanol, yielding 24.

### Synthesis of Core SubFormulae VIg-VIh

Core Formulae VIg-VIh are synthesized from fused-piperidines that are either commercially available or can be synthesized utilizing organic synthesis methods known to one of ordinary skill in the art (Houben-Weyl, Methods of Organic Synthesis, Thieme, Volume 21). One of ordinary skill in the art will know that further reactions of the core intermediate in series or in parallel will result in product aminoalkyl compounds of the present invention, as further shown in Examples 7-26.

### Synthesis of Core Formulae VIi-VIj

Core Formulae VIi-VIj can be prepared by reduction of a phthalimede to an isoindoline by either methods known in the literature to one of ordinary skill in the art or can be synthesized utilizing organic synthesis methods known to one of ordinary skill in the art. One of ordinary skill in the art will know that further reactions of the core intermediate in series or in parallel will result in product aminoalkyl compounds of the present invention, as further shown in Examples 7-26.

As an example of a synthesis method of core Formula VIi-VIj, 4-bromophthalimide (25) can be treated with a reducing agent (e.g., BF₃.OEt₂ followed by BH₃.THF) in an appropriate solvent (e.g., tetrahydrofuran, THF) for the appropriate length of time at the appropriate temperature, to yield 4-bromoisoindoline, 26. The appropriate temperature and appropriate length of time are determined by reference to Houben-Weyl 4th Ed. 1952, Methods of Organic Synthesis, Thieme, Volume 21.

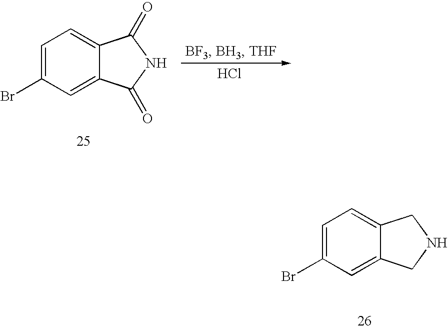

### Example 6

### Synthesis of Core Formula II

Core Formula II is synthesized from amides that are either commercially available or can be synthesized utilizing organic synthesis methods known to one of ordinary skill in the art (Houben-Weyl 4th Ed. 1952, Methods of Organic Synthesis, Thieme, Volume 21). Reactions of the core intermediate in series or in parallel results in product aminoalkyl compounds of the present invention, as further shown in Examples 7-26.

### Example 7

### General Synthesis Methods for Producing Aminoalkyl Compounds from Cores Shown in Examples 2-6

The intermediate amides prepared by the methods shown above can be further derivatized either on the amine or acid moiety. The derivatization can be done by a number of methods known to one of ordinary skill in the art, including Suzuki, cyanation, Buchwald-Hartwig, Molander and Stille-type coupling chemistry, but is not limited to these methods. (Metal-catalyzed Cross-coupling Reactions, ed. Francois Diederich and Peter J. Stang, Wiley-VCH, 1^{st} Edition, 1998 and Journal of Organic Chemistry 2003, 68:4302). All stereoisomers are envisioned as suitable starting materials, intermediates, and products.

For example, the 4-bromoisoindoline (26) can be coupled to a carboxylic acid such as (R)-Boc-phenylalanine (27) using a coupling agent (e.g., 1-hydroxybenzotriazole, HOBt) and a dehydrating agent (e.g., N-ethyl-N′-(3-dimethylaminopropyl)carbodiimide, EDC) and a base (e.g., diisopropylethylamine, DIPEA). The resulting amide (28) is then reacted with an appropriate palladium source (e.g., palladium dppf dichloride) and a trifluoroalkyl borate (e.g., potassium trans-styryltrifluoroborate, 29), a base (e.g., cesium carbonate) in an appropriate solvent system (e.g., water/THE) yielding 30.

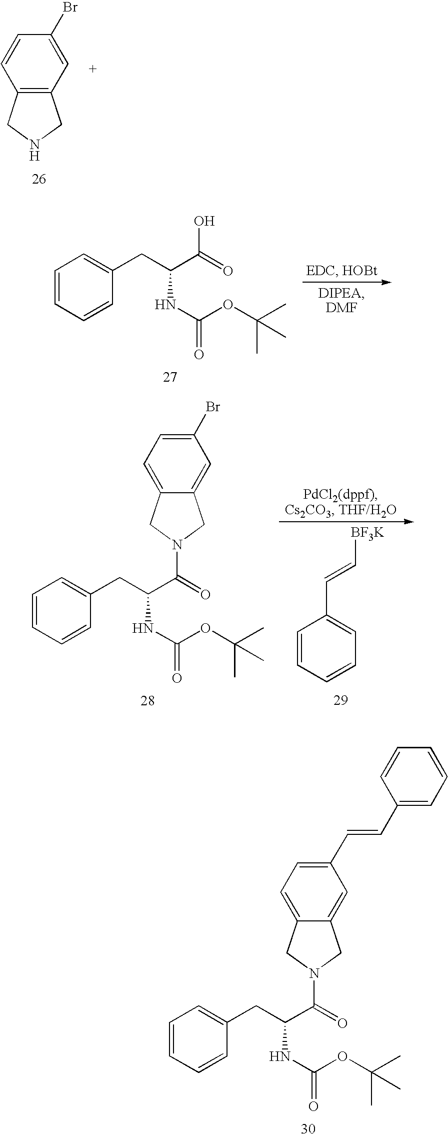

All core molecules were synthesized as described above. The compounds of the present invention that are prepared by the above listed methodologies are exemplified below but not limited to those protocols listed below.

### Example 8

### Preparation of (R)-2-Amino-1-(4-biphenyl-3-yl-3,6-dihydro-2H-pyridin-1-yl)-3-(4-chloro-phenyl)-propan-1-one

### 4-Trifluoromethanesulfonyloxy-3,6-dihydro-2H-pyridine-1-carboxylic acid tert-butyl ester

For synthesis of the ester, LiHMDS (20% in THF, 20 ml, 21 mmol) is added to a solution of 1-Boc-4-piperidone (2.79 g, 14 mmol) in dry THF (20 ml) at −78° C. under nitrogen. The mixture is stirred at −78° C. for 1 h. N-phenyltrifluoromethanesulfonimide (5.0 g, 14 mmol) is added as solid in one portion. The reaction mixture is stirred at −78° C. for 1 h. Then the mixture is warned up to room temperature over a period of 4 h. Saturated NaHCO₃ is added, and the aqueous solution is extracted with ethyl acetate two times (10 ml). The combined organic extracts are dried with Na₂SO₄, evaporated to dryness and the residue is purified by flash chromatography 0-15% ethyl acetate/hexane to afford product 4-trifluoromethanesulfonyloxy-3,6-dihydro-2H-pyridine-1-carboxylic acid tert-butyl ester as a yellow oil (3.86 g, 83%). The compound is carried onto the next step without purification.

### 4-Biphenyl-3-yl-3,6-dihydro-2H-pyridine-1-carboxylic acid tert-butyl ester

4-Trifluoromethanesulfonyloxy-3,6-dihydro-2H-pyridine-1-carboxylic acid tert-butyl ester (2.3 g, 6.98 mmol), LiCl (664 mg, 15.6 mmol), Pd(PPh₃)₄ (324 mg, 0.28 mmol), 3-biphenyl boronic acid (1.5 g, 7.5 mmol) are mixed in DME (10 ml). Na₂CO₃ (2M, 7 ml, 14 mmol) is added to the mixture and heated at 90° C. for 5 h. The black mixture is cooled and poured into water. The aqueous layer is extracted twice with ethyl acetate. The combined organic extracts are dried with Na₂SO₄. The black residue is purified by flash chromatography 0-20% ethyl acetate/hexane, yielding the desired product, 4-biphenyl-3-yl-3,6-dihydro-2H-pyridine-1-carboxylic acid tert-butyl ester as a yellow oil (1.7 g, 72%), (m/z 236 [MH⁺-Boc]).

### 4-Biphenyl-3-yl-1,2,3,6-tetrahydro-pyridine hydrochloride

4-Biphenyl-3-yl-1,2,3,6-tetrahydro-pyridine hydrochloride is produced by adding TFA (10 ml) to the solution of 4-biphenyl-3-yl-3,6-dihydro-2H-pyridine-1-carboxylic acid tert-butyl ester (2 g, 5.97 mmol) in DCM (30 ml). The resulting solution is stirred at room temperature for 4 h and evaporated to dryness. The resulting oil is dissolved in MeOH, and 4M hydrochloric acid in dioxane is added and the mixture is evaporated to dryness. The resulting yellow solid is washed with ether and dried under reduced pressure (1.5 g, 93%), (m/z 236 [MH+]).

### [(R)-2-(4-Biphenyl-3-yl-3,6-dihydro-2H-pyridin-1-yl)-1-(4-chloro-benzyl)-2-oxo-ethyl]-carbamic acid tert-butyl ester

To the solution of 4-biphenyl-3-yl-1,2,3,6-tetrahydro-pyridine hydrochloride (169 mg, 0.62 mmol) in DMF (5 ml) is added 1-ethyl-3-(3′-(dimethylamino)propyl)carbodiimide hydrochloride (EDC, 120 mg, 0.63), 1-hydroxybenzotriazole (HOBt, 110 mg, 0.81 mmol), and Boc-4-chloro-D-phenylalaine (185 mg, 0.62 mmol). Diisopropylethylamine (DIPEA, 0.54 ml, 3.1 mmol) is added and the resulting solution is stirred at room temperature for 16 h. The mixture is then poured into water and the water solution is extracted twice with ethyl acetate. The combined organic solution is dried with Na₂SO₄ and evaporated to dryness. The residue is purified by flash chromatography 10%-25% ethyl acetate/hexane to give [(R)-2-(4-biphenyl-3-yl-3,6-dihydro-2H-pyridin-1-yl)-1-(4-chloro-benzyl)-2-oxo-ethyl]-carbamic acid tert-butyl ester as light yellow oil (174.9 mg, 68%).

### (R)-2-Amino-1-(4-biphenyl-3-yl-3,6-dihydro-2H-pyridin-1-yl)-3-(4-chloro-phenyl)propan-1-one

To a solution of [(R)-2-(4-biphenyl-3-yl-3,6-dihydro-2H-pyridin-1-yl)-1-(4-chloro-benzyl)-2-oxo-ethyl]-carbamic acid tert-butyl ester (174.9 mg, 0.42 mmol) in DCM (10 ml) is added TFA (5 ml). The mixture is stirred at room temperature for 4 h and evaporated to dryness. The resulting oil is dissolved in MeOH, then 4M hydrochloric acid in dioxane is added and the mixture is evaporated to dryness. The resulting white solid is washed with diethyl ether and dried under reduced pressure to yield (R)-2-Amino-1-(4-biphenyl-3-yl-3,6-dihydro-2H-pyridin-1-yl)-3-(4-chloro-phenyl)propan-1-one (135.1 mg, 77.3%), (m/z 417 [MH+]).

### Example 9

### Preparation of (R)-2-Amino-1-(4-biphenyl-3-yl-3,6-dihydro-2H-pyridin-1-yl)-3-(4-chloro-phenyl)-propan-1-one

### 4-Biphenyl-3-yl-piperidine hydrochloride

A catalytic amount of Pd/C (10%) is added to a solution of 4-biphenyl-3-yl-1,2,3,6-tetrahydro-pyridine hydrochloride (1.5 g, 5.5 mmol) in MeOH (20 ml) and the mixture is placed in a Parr shaker. The hydrogenation is done at 60 psi over 4 h. 4-Biphenyl-3-yl-piperidine hydrochloride is obtained as an off white solid (1.5 g, 99.9%) by filtration and evaporation the solvent, (m/z 238 [MH+]).

### [(R)-2-(4-Biphenyl-3-yl-piperidin-1-yl)-1-(4-chloro-benzyl)-2-oxo-ethyl]-carbamic acid tert-butyl ester

To a solution of 4-biphenyl-3-yl-piperidine hydrochloride (140 mg, 0.51 mmol) in DMF (5 ml) is added each of EDC (108.14 mg, 0.56 mmol), HOBt (103.4 mg, 0.77 mmol) and Boc-4-chloro-D-phenylalaine (152.9 mg, 0.51 mmol). DIPEA (0.44 ml, 2.5 mmol) is added and the resulting solution is stirred at room temperature for 16 h. The resulting mixture is poured into water and the water solution is extracted with ethyl acetate. The combined organic layers are dried with Na₂SO₄ and evaporated to dryness. The resulting residue is purified by flash chromatography 10%-25% ethyl acetate in hexane to give [(R)-2-(4-biphenyl-3-yl-piperidin-1-yl)-1-(4-chloro-benzyl)-2-oxo-ethyl]-carbamic acid tert-butyl ester as a yellow oil (188.8 mg, 71%) (m/z 519 [MH+]).

### (R)-2-Amino-1-(4-biphenyl-3-yl-piperidin-1-yl)-3-(4-chloro-phenyl)-propan-1-one hydrochloride

To a solution of [(R)-2-(4-biphenyl-3-yl-piperidin-1-yl)-1-(4-chloro-benzyl)-2-oxo-ethyl]-carbamic acid tert-butyl ester (166 mg, 0.32 mmol) in DCM (10 ml) is added TFA (5 ml). The mixture is stirred at room temperature for 3 h and the resulting mixture is evaporated to dryness. The residue is dissolved in MeOH, 4 M hydrochloric acid in dioxane is added and the mixture is evaporated to dryness. The obtained white solid is washed with ether and dried under reduced pressure to yield (R)-2-Amino-1-(4-biphenyl-3-yl-piperidin-1-yl)-3-(4-chloro-phenyl)-propan-1-one hydrochloride (124.2 mg, 85%) (m/z 419 [MH+]).

### Example 10

### Preparation (R)-2-Amino-3-(2,4-dichloro-phenyl)-1-[4-(pyridin-2-ylsulfanyl)-piperidin-1-yl]-propan-1-one

### (R)-2-Butyl oxycarbonyl-amino-3-(2,4-dichloro-phenyl)-1-[4-(pyridin-2-ylsulfanyl)-piperidin-1-yl]-propan-1-one

2-(Piperidin-4-ylsulfanyl)pyridine.2HCl (50 mg, 0.15 mmol) and (R)-2-tert-butoxycarbonylamino-3-(2,4-dichloro-phenyl)-propionic acid (40 mg, 0.15 mmol) is dissolved in DMF (1 ml). HOBt (30 mg, 0.21 mmol), DIPEA (1.2 equivalent) and EDC (28 mg, 0.15 mmol) are added and the mixture is stirred for three days, and is concentrated in vacuo. The resulting viscous oil is filtered through an silica plug, yielding (R)-2-Butyl oxycarbonyl-amino-3-(2,4-dichloro-phenyl)-1-[4-(pyridin-2-ylsulfanyl)-piperidin-1-yl]-propan-1-one (40 mg, 52%), (m/z 510 [MH+]).

### (R)-2-Amino-3-(2,4-dichloro-phenyl)-1-[4-(pyridin-2-ylsulfanyl)-piperidin-1-yl]-propan-1-one

(R)-2-Butyl oxycarbonyl-amino-3-(2,4-dichloro-phenyl)-1-[4-(pyridin-2-ylsulfanyl)-piperidin-1-yl]-propan-1-one is taken up in 2 ml MeOH, to which is added 2 ml 1N HCl in diethyl ether. The mixture is stirred for 8 h, then concentrated in vacuo, then triturated with diethyl ether (three times) and ethyl acetate (once), yielding (R)-2-Amino-3-(2,4-dichloro-phenyl)-1-[4-(pyridin-2-ylsulfanyl)-piperidin-1-yl]-propan-1-one (33 mg, 87%), (m/z 412-[MH+]).

### Example 11

### Preparation of (R)-2-Amino-3-(2,4-dichloro-phenyl)-1-[4-(2-trifluoromethyl-phenoxy)-piperidin-1-yl]-propan-1-one

### (R)-{1-(2,4-Dichloro-benzyl)-2-oxo-2-[4-(2-trifluoromethyl-phenoxy-piperidin-1-yl]-ethyl}-carbamic acid tert-butyl ester

4-[2-(trifluoromethyl)phenoxy)piperidine (42 mg, 0.15 mmol) and (R)-2-tert-butoxycarbonylamino-3-(2,4-dichloro-phenyl)-propionic acid (50 mg, 0.15 mmol) is dissolved in 1 ml DMF. HOBt (30 mg, 0.21 mmol), DIPEA (1.2 equivalent) and EDC (28 mg, 0.15 mmol) are added and the mixture is stirred for three days, and is concentrated in vacuo. The resulting viscous oil is filtered through an silica plug, yielding (R)-{1-(2,4-Dichloro-benzyl)-2-oxo-2-[4-(2-trifluoromethyl-phenoxy)-piperidin-1-yl]-ethyl}-carbamic acid tert-butyl ester (65 mg, 77%), (m/z 561 [MH+]).

### (R)-2-Amino-3-(2,4-dichloro-phenyl)-1-[4-(2-trifluoromethyl-phenoxy)-piperidin-1-yl]-propan-1-one

(R)-{1-(2,4-Dichloro-benzyl)-2-oxo-2-[4-(2-trifluoromethyl-phenoxy)-piperidin-1-yl]-ethyl}-carbamic acid tert-butyl ester is taken up in 2 ml MeOH, to which is added 2 ml 1N HCl in diethyl ether. The mixture is stirred for 8 h, then concentrated in vacuo, then triturated with diethyl ether (three times) and with ethyl acetate (once), yielding (R)-2-Amino-3-(2,4-dichloro-phenyl)-1-[4-(2-trifluoromethyl-phenoxy)-piperidin-1-yl]-propan-1-one (57 mg, 99%), (m/z 461 MH+).

### Example 12

### Preparation of (R)-2-Amino-1-[4-(5-chloro-2-methyl-phenyl)-piperazin-1-yl]-3-(2,4-dichloro-phenyl)-propan-1-one

(R)-2-[4-(5-Chloro-2-methyl-phenyl)-piperazin-1-yl]-1-(2,4-dichloro-benzyl)-2-oxo-ethyl-carbamic acid tert-butyl ester

(R)-2-tert-Butoxycarbonylamino-3-(2,4-dichloro-phenyl)-propionic acid (150 mg, 0.712 mmol) is dissolved in a solution of EDC (151 mg, 0.783 mmol) and HOBt (144 mg, 1.067 mmol) in 5 ml DMF, and 1-(5-Chloro-2-methyl-phenyl)-piperazine (237.6 mg, 0.712 mmol) is added to the reaction mixture followed by DIPEA (459.8 mg, 3.55 mmol). A clear reaction solution is obtained and stirred at room temperature for 8 h, and the product is then extracted with ethyl acetate (25 ml). The organic layer is washed with saturated NaHCO₃ and saturated NaCl solution (15 ml each), and the organic phase is then separated, dried with Na₂SO₄ and evaporated in vacuo resulting in [(R)-2-[4-(5-Chloro-2-methyl-phenyl)-piperazin-1-yl]-1-(2,4-dichloro-benzyl)-2-oxo-ethyl]-carbamic acid tert-butyl ester (262.4 mg, 69.8%), (m/z 528 [MH⁺]).

### (R)-2-Amino-1-[4-(5-chloro-2-methyl-phenyl)-piperazin-1-yl]-3-(2,4-dichloro-phenyl)-propan-1-one

Trifluoroacetic acid (2 ml) is added to a solution of [(R)-2-[4-(5-Chloro-2-methyl-phenyl)-piperazin-1-yl]-1-(2,4-dichloro-benzyl)-2-oxo-ethyl]-carbamic acid tert-butyl ester (250 mg, 0.474 mmol) in 5 ml DCM. The resulting solution is stirred at room temperature for 3 h and evaporated to dryness. The resulting residue is dissolved in 4M HCl in dioxane (3 ml) and stirred at room temperature for 8 h. The reaction is concentrated under reduced pressure and dissolved in ethyl acetate (3 ml). Hexane (5 ml) is added and the product compound precipitated out of the solution. The resulting suspension is filtered, the collected solid is washed with hexane (3 ml) and dried in vacuo, producing 115 mg (56.9%) of (R)-2-Amino-1-[4-(5-chloro-2-methyl-phenyl)-piperazin-1-yl]-3-(2,4-dichloro-phenyl)-propan-1-one, (m/z 428 [MH⁺]).

### Example 13

### Preparation of (R)-2-Amino-3-(3-chloro-phenyl)-1-[4-(5-fluoro-2-trifluoromethyl-benzoyl)-piperazin-1-yl]-propan-1-one

### (5-Fluoro-2-trifluoromethyl-phenyl)-piperazin-1-yl-methanone

Triethylamine (0.814 g, 8.04 mmol) is added to the solution of piperazine-1-carboxylic acid tert-butyl ester (1.21 g, 5.3 6 mmol) in ethyl acetate (10 ml) at room temperature. 5-Fluoro-2-trifluoromethyl-benzoyl chloride (1 g, 5.36 mmol) is added to the resulting solution and the mixture is stirred at room temperature for 8 h. The reaction is diluted by adding water (15 ml) and ethyl acetate (15 ml). The organic phase is separated, dried with Na₂SO₄ and evaporated in vacuo to afford 1.61 g of 4-(5-Fluoro-2-trifluoromethyl-benzoyl)-piperazine-1-carboxylic acid tert-butyl ester. Trifluoroacetic acid (2 ml) is added to the solution of 4-(5-Fluoro-2-trifluoromethyl-benzoyl)-piperazine-1-carboxylic acid tert-butyl ester (1.61 g, 4.28 mmol) in 5 ml DCM. The resulting solution is stirred at room temperature for 3 hrs and evaporated to dryness. The resulting residue is dissolved in 3 ml 4M HCl in dioxane and stirred at room temperature for 8 h. The reaction is dried in vacuo, producing 1.18 g of (5-Fluoro-2-trifluoromethyl-phenyl)-piperazin-1-yl-methanone, (m/z 277 [MH⁺]).

### {(R)-1-(3-Chloro-benzyl)-2-[4-(5-fluoro-2-trifluoromethyl-benzoyl)-piperazin-1-yl]-2-oxo-ethyl}-carbamic acid tert-butyl ester

(R)-2-tert-Butoxycarbonylamino-3-(3-chloro-phenyl)-propionic acid (130 mg, 0.434 mmol) is dissolved in a solution of EDC (91 mg, 0.477 mmol), HOBt (88 mg, 0.651 mmol) in 3 ml DMF. (5-Fluoro-2-trifluoromethyl-phenyl)-piperazin-1-yl-methanone (120 mg, 0.434 mmol) is added to the reaction mixture followed by DIPEA (280 mg, 2.17 mmol) and the mixture is stirred at room temperature for 8 h. The product is extracted with ethyl acetate (25 ml), and the organic layer is washed with saturated NaHCO₃ and saturated NaCl solution (15 ml each). The organic phase is separated, dried over Na₂SO₄ and evaporated in vacuo to afford 170 mg of {(R)-1-(3-Chloro-benzyl)-2-[4-(5-fluoro-2-trifluoromethyl-benzoyl)-piperazin-1-yl]-2-oxo-ethyl}-carbamic acid tert-butyl ester, (m/z 556.1 [MH⁻]).

### (R)-2-Amino-3-(3-chloro-phenyl)-1-[4-(5-fluoro-2-trifluoromethyl-benzoyl)-piperazin-1-yl]-propan-1-one

Trifluoroacetic acid (2 ml) is added to the solution of {(R)-1-(3-Chloro-benzyl)-2-[4-(5-fluoro-2-trifluoromethyl-benzoyl)-piperazin-1-yl]-2-oxo-ethyl}-carbamic acid tert-butyl ester (170 mg, 0.304 mmol) in DCM (5 ml). The resulting solution is stirred at room temperature for 3 hrs and evaporated to dryness. The resulting residue is dissolved in 4M HCl in dioxane (3 ml) and stirred at room temperature for 8 h. The reaction is concentrated under reduced pressure and dissolved in 3 ml ethyl acetate. Hexane (5 ml) is added and the product is precipitated out of the solution. The resulting suspension is filtered, the collected solid is washed with hexane (3 ml) and dried in vacuo, producing 83.5 mg (60.0%) of (R)-2-Amino-3-(3-chloro-phenyl)-1-[4-(5-fluoro-2-trifluoromethyl-benzoyl)-piperazin-1-yl]-propan-1-one, (m/z 458.1 [MH⁺]).

### Example 14

### Preparation of (R)-1-Methylspiro[indole-3,4′-piperidine]-2(1H)-one, 1′-[2-amino-1-oxo-3-(2,4-dichlorophenyl)propyl]

1-Methylspiro[indole-3,4′-piperidine]-2(1H)-one (50 mg, 0.23 mmol) and (R)-2-tert-butoxycarbonylamino-3-(2,4-dichloro-phenyl)-propionic acid (69 mg, 0.23 mmol) are dissolved in 2.5 ml DMF and HOBt (45 mg, 0.33 mmol), DIPEA (0.20 ml, 1.2 mmol) and EDC (0.44 g, 0.23 mmol) are stirred overnight. Brine is added and the resulting mixture is extracted three times with ethyl acetate. The reaction is further purified by column chromatography with 10-25% ethyl acetate in hexane, to yield the Boc-derivative of the title compound (34 mg, 28%). To the Boc-derivative of (R)-1-Methylspiro[indole-3,4′-piperidine]-2(1H)-one, 1′-[2-amino-1-oxo-3-(2,4-dichlorophenyl)propyl] is added MeOH (5 ml) and 1.0 M HCl in diethyl ether (3 ml) and DCM. The mixture is stirred overnight, then dried in vacuo to yield (R)-1-Methylspiro[indole-3,4′-piperidine]-2(1H)-one, 1′-[2-amino-1-oxo-3-(2,4-dichlorophenyl)propyl] (29 mg, quantitative yield), (m/z 432 [MH+]).

### Example 15

### Preparation (R)-3,4-Dihydrospiro[2H-1-benzopyran-2,4′-piperidine], 1′-[2-amino-1-oxo-3-(2,4-dichlorophenyl)propyl]

3,4-Dihydrospiro[2H-1-benzopyran-2,4′-piperidine] (150 mg, 0.72 mmol) and (R)-2-tert-butoxycarbonylamino-3-(2,4-dichloro-phenyl)-propionic acid (222 mg, 0.65 mmol) is dissolved in 3 ml DMF. TPTU (236 mg, 0.80 mmol) and NMM (0.24 ml, 2.7 mmol) are added and the resulting mixture is stirred overnight. The crude reaction mix is concentrated under reduced pressure. Column chromatography with 10-25% ethyl acetate in hexane yields the Boc-derivative of (R)-3,4-Dihydrospiro[2H-1-benzopyran-2,4′-piperidine], 1′-[2-amino-1-oxo-3-(2,4-dichlorophenyl)propyl] (316 mg, 93%), (m/z 463 [MH+]). The product is dissolved in MeOH (3 ml) and 4.0 M HCl in dioxane (1 ml) is added, and the mixture stirs overnight. The vial is evacuated to yield (R)-3,4-Dihydrospiro[2H-1-benzopyran-2,4′-piperidine], 1′-[2-amino-1-oxo-3-(2,4-dichlorophenyl)propyl] (227 mg, 91%), (m/z 419 [MH+])).

### Example 16

### Preparation of (R)-Spiro[1H-indene-1,4′-piperidine], 1′-[2-amino-1-oxo-3-(2,4-dichlorophenyl)propyl]-2,3-dihydro

### Spiro[indan-1,4′-piperidine]

N-Boc spiro[1H-indene-1,4′-piperidine] (700 mg, 2.45 mmol) is added to ethanol (50 ml) in a Parr-shaker and hydrogenated over Pd/C (10%) for 2 h. The resulting reaction mixture is filtered through Celite and concentrated to dryness. The crude product is used without further purification for the next step.

### (R)-Spiro[1H-indene-1,4′-piperidine], 1′-[2-amino-1-oxo-3-(2,4-dichlorophenyl)propyl]-2,3-dihydro

The Spiro[indan-1,4′-piperidine] compound (50 mg, 0.22 mmol) is dissolved in DMF (3 ml), then (R)-2-tert-Butoxycarbonylamino-3-(2,4-dichloro-phenyl)-propanoic acid (74 mg, 0.22 mmol), EDC (43 mg, 0.22 mmol), DIPEA (0.200 ml, 1.1 mmol), HOBt (45 mg, 0.33 mmol) are all added. The reaction is stirred for 24 h. The reaction mixture is diluted with saturated sodium bicarbonate solution (10 ml) and extracted three times with ethyl acetate (10 ml). The organic layer is concentrated in vacuo, then purified by column chromatography (10-25% ethyl acetate in hexane) yielding the Boc-derivative of (R)-Spiro[1H-indene-1,4′-piperidine], 1′-[2-amino-1-oxo-3-(2,4-dichlorophenyl)propyl]-2,3-dihydro (93 mg, quantitative yield), (m/z 403 [MH+]), an orange oil (105 mg, 93%) (m/z 447 [MH+]). To the Boc-derivative of (R)-Spiro[1H-indene-1,4′-piperidine], 1′-[2-amino-1-oxo-3-(2,4-dichlorophenyl)propyl]-2,3-dihydro (93 mg, quantitative yield), (m/z 403 [MH+]), is added MeOH (3 ml) and 4.0M HCl in dioxane (1 ml). The resulting mixture is allowed to stir overnight. The reaction mixture is evaporated to dryness to yield (R)-Spiro[1H-indene-1,4′-piperidine], 1′-[2-amino-1-oxo-3-(2,4-dichlorophenyl)propyl]-2,3-dihydro (93 mg, quantitative yield), (m/z 403 [MH+]).

### Example 17

### Preparation of 2-Amino-3-(3-chloro-biphenyl-4-yl)-1-(1,3-dihydro-isoindol-2-yl)-propan-1-one

### 4-Bromo-1-bromomethyl-2-chloro-benzene

To a solution of 2-Chloro 4-bromo toluene (5.0 g, 24.0 mmol) in carbon tetrachloride is added N-bromo succinimide (4.42 g, 24.8-mmol) and benzoyl peroxide (600 mg, 2.5 mmol) and the reaction is heated at reflux for 18 h. The reaction is allowed to cool to ambient temperature and is extracted saturated Na₂CO₃ solution and brine (10 ml each). The organic layer is separated and concentrated in vacuo. The residue is purified by column chromatography eluting with 100% hexanes resulting in 4-Bromo-1-bromomethyl-2-chloro-benzene, as a colorless oil (5.5 g, 80%). 4-Bromo-1-bromomethyl-2-chloro-benzene is analyzed by NMR.

### 2-(Benzhydrylidene-amino)-1-(1,3-dihydro-isoindol-2-yl)-ethanone

To a solution of isoindoline (690 mg, 5.79 mmol) in DCM (5 ml) is added a 2M solution of trimethyl aluminum in heptane (2.9 ml, 5.79 mmol) and the resulting solution is allowed to stir at ambient temperature for 30 minutes. To this solution is added a solution of (benzhydrylidene-amino)-acetic acid ethyl ester (1.29 g, 4.82 mmol) in DCM (5 ml). The reaction is allowed to stir at ambient temperature for 18 h and carefully quenched with a 10% aqueous solution of citric acid. The resulting biphasic mixture is diluted with DCM (10 ml) and extracted with a saturated solution of Rochelle's Salt (10 ml). The organic layer is separated, concentrated in vacuo and purified by column chromatography (hexanes-ethyl acetate 7-70%) resulting in 2-(Benzhydrylidene-amino)-1-(1,3-dihydro-isoindol-2-yl)-ethanone (1.0 g, 61%). 2-(Benzhydrylidene-amino)-1-(1,3-dihydro-isoindol-2-yl)-ethanone is analyzed by HPLC/Mass Spec.

### 2-(Benzhydrylidene-amino)-3-(4-bromo-2-chloro-phenyl)-1-(1,3-dihydro-isoindol-2-yl)-propan-1-one

2-(Benzhydrylidene-amino)-1-(1,3-dihydro-isoindol-2-yl)-ethanone (200 mg, 0.58 mmol), 4-Bromo-1-bromomethyl-2-chloro-benzene (250 mg, 0.87 mmol), potassium hydroxide (320 mg, 5.8 mmol) and tetrabutyl ammonium bromide (20 mg, 0.058 mmol) are suspended in DCM and vigorously stirred for 2 h. The reaction is quenched with a 10% aqueous solution of citric acid. The organic phase is separated and concentrated in vacuo and the residue is purified by column chromatography (hexanes-ethyl acetate 7-60%) resulting in 2-(Benzhydrylidene-amino)-1-(1,3-dihydro-isoindol-2-yl)-ethanone (300 mg, 95%). 2-(Benzhydrylidene-amino)-1-(1,3-dihydro-isoindol-2-yl)-ethanone is analyzed by NMR and HPLC/Mass Spec.

### 2-(Benzhydrylidene-amino)-3-(3-chloro-biphenyl-4-yl)-1-(1,3-dihydro-isoindol-2-yl)-propan-1-one

2-(Benzhydrylidene-amino)-3-(4-bromo-2-chloro-phenyl)-1-(1,3-dihydro-isoindol-2-yl)-propan-1-one (100 mg, 0.18 mmol), phenyl boronic acid (33 mg, 0.27 mmol) and sodium carbonate (85 mg, 0.81 mmol) are dissolved in 2:1 dioxane-water (1.5 ml), palladium tetrakis triphenylphosphine (12 mg, 0.018 mmol) is added and the reaction is heated to reflux for 18 h. The reaction is partitioned between DCM (10 ml) and brine (10 ml), the organic layer is separated and concentrated in vacuo and the residue is purified by column chromatography (ethyl acetate-hexane 5-50%) resulting in the protected biphenyl alanine (100 mg, 100%). 2-(Benzhydrylidene-amino)-3-(3-chloro-biphenyl-4-yl)-1-(1,3-dihydro-isoindol-2-yl)-propan-1-one is analyzed by HPLC/Mass Spec.

### 2-Amino-3-(3-chloro-biphenyl-4-yl)-1-(1,3-dihydro-isoindol-2-yl)-propan-1-one

A solution of 2-(benzhydrylidene-amino)-3-(3-chloro-biphenyl-4-yl)-1-(1,3-dihydro-isoindol-2-yl)-propan-1-one (100 mg, 0.18 mmol) in DCM (3 ml) is treated with a 4N solution of HCl in dioxane (0.2 ml) and allowed to stir at ambient temperature for 18 h. The precipitated product is collected by filtration and washed with DCM resulting in 2-Amino-3-(3-chloro-biphenyl-4-yl)-1-(1,3-dihydro-isoindol-2-yl)-propan-1-one as an HCl salt (3 mg), (m/z 377.4 [MH+]).

### Example 18

### Preparation of 2-Amino-3-(2-chloro-3,4-dimethoxy-phenyl)-1-(1,3-dihydro-isoindol-2-yl)-propan-1-one

### 2-tert-Butoxycarbonylamino-3-(2-chloro-3,4-dimethoxy-phenyl)-acrylic acid methyl ester

N-Boc-alpha-phosphono glycine trimethyl ester (750 mg, 2.52 mmol) is dissolved in DCM (10 ml) and treated with DBU (365 mg, 2.39 mmol) and allowed to stir at ambient temperature for 30 minutes. A solution of 2-chloro 3,4 dimethoxy benzaldehyde (455 mg, 2.27 mmol) in DCM (2 ml) is added and the resulting mixture is allowed to stir at ambient temperature for 18 h. The reaction mixture is loaded directly onto a silica gel column and eluted with a gradient of hexanes-ethyl acetate (5-40%) resulting in (640 mg, 76%) 2-tert-Butoxycarbonylamino-3-(2-chloro-3,4-dimethoxy-phenyl)-acrylic acid methyl ester. 2-tert-Butoxycarbonylamino-3-(2-chloro-3,4-dimethoxy-phenyl)-acrylic acid methyl ester is analyzed by NMR and HPLC/Mass Spec.

### 2-tert-Butoxycarbonylamino-3-(2-chloro-3,4-dimethoxy-phenyl)-propionic acid methyl ester

To a solution of 2-tert-butoxycarbonylamino-3-(2-chloro-3,4-dimethoxy-phenyl)-acrylic acid methyl ester in ethanol (50 ml) is added platinum oxide (30 mg). The resulting suspension is stirred in a hydrogen atmosphere (1 atm) for 90 minutes. The suspension is filtered through a pad of Celite and concentrated in vacuo resulting in 2-tert-Butoxycarbonylamino-3-(2-chloro-3,4-dimethoxy-phenyl)-propionic acid methyl ester (520 mg, 81%). 2-tert-Butoxycarbonylamino-3-(2-chloro-3,4-dimethoxy-phenyl)-propionic acid methyl ester is analyzed by NMR and HPLC/Mass Spec.

### 2-tert-Butoxycarbonylamino-3-(2-chloro-3,4-dimethoxy-phenyl)-1-(1,3-dihydro-isoindol-2-yl)-propan-1-one

To a solution of isoindoline (335 mg, 2.8 mmol) in DCM (5 ml) is added a 2M solution of trimethylaluminum in toluene (1.4 ml, 2.8 mmol) and the resulting solution is allowed to stir at ambient temperature for 30 minutes and is then added to a solution of 2-tert-butoxycarbonylamino-3-(2-chloro-3,4-dimethoxy-phenyl)-propionic acid methyl ester (520 mg, 1.39 mmol) in DCM (5 ml). The reaction is allowed to stir at ambient temperature for 18 h and carefully quenched with a 10% aqueous solution of citric acid. The resulting biphasic mixture is diluted with DCM (20 ml) and extracted with a saturated solution of Rochelle's Salt (20 ml). The organic layer is separated, concentrated in vacuo and purified by column chromatography (hexanes-ethyl acetate 15-60%) resulting in 2-tert-Butoxycarbonylamino-3-(2-chloro-3,4-dimethoxy-phenyl)-1-(1,3-dihydro-isoindol-2-yl)-propan-1-one (300 mg, 47%). 2-tert-Butoxycarbonylamino-3-(2-chloro-3,4-dimethoxy-phenyl)-1-(1,3-dihydro-isoindol-2-yl)-propan-1-one is analyzed by HPLC/Mass Spec.

### 2-Amino-3-(2-chloro-3,4-dimethoxy-phenyl)-1-(1,3-dihydro-isoindol-2-yl)-propan-1-one

A solution of Boc-isoindoline amide (100 mg, 0.22 mmol) in DCM (2.5 ml) is treated with a 4N solution of HCl in dioxane (0.3 ml) and allowed to stir at ambient temperature for 18 h. The precipitated product is collected by filtration and washed with DCM, resulting in 2-Amino-3-(2-chloro-3,4-dimethoxy-phenyl)-1-(1,3-dihydro-isoindol-2-yl)-propan-1-one as an HCl salt (80 mg, 92%). 2-Amino-3-(2-chloro-3,4-dimethoxy-phenyl)-1-(1,3-dihydro-isoindol-2-yl)-propan-1-one is analyzed by NMR and HPLC/Mass Spec. (m/z 361.4 [MH+]).

### Example 19

### Preparation of (R)-2-Amino-3-(2,4-dichloro-phenyl)-1-(2,6-dihydro-4H-pyrrolo[3,4-c]pyrazol-5-yl)-propan-1-one

The dihydro-pyrrolo-pyrazole is synthesized according to the method described in Heterocycles 2002, 56:257. This (76.06 mg, 0.42 mmol) is coupled to Boc-(D)-2,4-dichlorophenyl alanine (147.1 mg, 0.42 mmol) using EDC, HOBt, DIPEA, purified by HPLC (177% yield) which is then (30 mg, 0.07 mmol) deprotected with HCl according to the method described above and purified by HPLC to yield 40% (R)-2-Amino-3-(2,4-dichloro-phenyl)-1-(2,6-dihydro-4H-pyrrolo[3,4-c]pyrazol-5-yl)-propan-1-one (m/z 325 [MH+]).

### Example 20

### Preparation of (R)-2-Amino-3-(2,4-dichloro-phenyl)-1-(5,7-dihydro-pyrrolo[3,4-d]pyrimidin-6-yl)-propan-1-one

The dihydro-pyrrolo-pyrimidine is synthesized according to the method described in Heterocycles 2002, 56:257. Dihydro-pyrrolo-pyrimidine (20 mg, 0.104 mg) is then added to a solution of Boc-(D)-2,4-dichlorophenyl alanine (34.8 mg, 0.104 mmol), O-(7-azabenzotriazol-1-yl)-N,N,N′,N′-tetramethyluronium hexafluorophosphate (HATU, 47.5 mg, 0.125 mmol) and DIPEA (0.34 ml, 0.624 mmol) in 2 ml DCM and stirred for four hours, then concentrated in vacuo. The Boc derivative of (R)-2-Amino-3-(2,4-dichloro-phenyl)-1-(5,7-dihydro-pyrrolo[3,4-d]pyrimidin-6-yl)-propan-1-one is subsequently deprotected with HCl according to the general method shown in Example 19. The compound is then purified by HPLC to yield (R)-2-Amino-3-(2,4-dichloro-phenyl)-1-(5,7-dihydro-pyrrolo[3,4-d]pyrimidin-6-yl)-propan-1-one (41%), (m/z 337 [MH+]).

### Example 21

### Preparation of 5-bromoisoindoline

5-bromoisoindoline-1,3-dione (5 g, 22.1 mmol) is dissolved in anhydrous THF (30 ml), treated with BF₃.OEt₂ (6.667 ml, 132.7 mmol) is added and the reaction was stirred at ambient temperature for 30 minutes. To the reaction mixture 1.0M BH₃.THF complex (176.94 ml, 525.3 mmol) is added and the reaction is heated to 40° C. for 36 h. The progress of the reaction is followed by LC/MS. After completion, the reaction mixture is cooled to ambient temperature and quenched with MeOH (6 ml, drop wise) until the bubbling ceases. Then 2N HCl in water (˜40 ml, 80 mmol) are added and the mixture is refluxed for 3 h. The reaction is then cooled to ambient temperature and is washed with diethyl ether (2×40 ml). The water layer is brought to pH 14 with 6N NaOH (aq) and extracted with ethyl acetate (3×100 ml). The combined organic extracts are dried over anhydrous Na₂SO₄ and solvent is removed under reduced pressure to yield 5-bromoisoindoline (68%), (m/z 413 [MH+]).

### Example 22

### Preparation of (R)-2-Amino-3-(2,4-dichloro-phenyl)-1-(5,7-dihydro-pyrrolo[3,4-b]pyridin-6-yl)-propan-1-one

Pyrrolo[3,4-b]pyridine-5,7-dione (0.536 g, 3.619 mmol) is reduced according to the representative protocol for the 5-bromoisoindoline reduction shown in Example 21, yielding 25% of the crude product. This (50 mg, 0.416 mmol) is then coupled to Boc-(D)-2,4-dichlorophenyl alanine (139.03 mg, 0.416 mmol) using HATU (as shown for (R)-2-Amino-3-(2,4-dichloro-phenyl)-1-(5,7-dihydro-pyrrolo[3,4-d]pyrimidin-6-yl)-propan-1-one, purified by HPLC (28% yield) and subsequently deprotected with HCl following the general coupling protocols shown in Example 19. Purification is done by HPLC to yield (R)-2-Amino-3-(2,4-dichloro-phenyl)-1-(5,7-dihydro-pyrrolo[3,4-b]pyridin-6-yl)-propan-1-one (54%), (m/z 336 [MH+]).

### Example 23

### Preparation of 3-(4-Allyl-2-chloro-phenyl)-2-amino-1-(1,3-dihydro-isoindol-2-yl)-propan-1-one

To a solution of the aryl bromide (86 mg, 0.179 mmol) in DMF (4 ml) is added allyltributyl tin (0.055 ml, 0.181 mmol), LiCl (2 mg, 0.044 mmol) and [Pd(PPh₃)₂Cl₂] (7.08 mg). The suspension is stirred at 90° C. overnight, then cooled to room temperature and diluted with saturated NaHCO₃ (10 ml) followed by extraction with ether (50 ml×3). The diethyl ether layer is washed with brine (20 ml) and dried over sodium sulfate. The solvent is removed in vacuo and the crude product is purified by silica gel column chromatography with 0 to 40% ethyl acetate in hexane to provide the Boc-protected coupled product (71%). The compound thus obtained (55.919 mg, 0.127 mmol) is then deprotected as described in Example 19 using TFA in DCM and purified by HPLC to yield 3-(4-Allyl-2-chloro-phenyl)-2-amino-1-(1,3-dihydro-isoindol-2-yl)-propan-1-one (73%), (m/z 340 [MH+]).

### Example 24

### Preparation of (R)-2-Amino-3-(2,4-dichloro-phenyl)-1-spiroindene

The spiro-indene is synthesized according to the method described in Journal of Medicinal Chemistry 1992, 35(21):3919. This amine (95 mg, 0.043 mmol) is coupled to Boc-(D)-2,4-dichlorophenyl alanine (143.7 mg, 0.43 mmol), purified by column chromatography on silica gel (hexane/ethyl acetate 4:1) as described above to provide the coupled compound (74%). This (159 mg, 0.31 mmol) is then deprotected in a solution of MeOH using HCl in dioxane as described above to give the deprotected compound (quantitative), (m/z 400 [MH+]).

### Example 25

### Preparation of 1-{2-[(R)-2-Amino-3-(2,4-dichloro-phenyl)-propionyl]-2,3-dihydro-1H-isoindol-5-ylmethyl}-3-(4-dimethylamino-phenyl)-urea

### 2-[(R)-2-Amino-3-(2,4-dichloro-phenyl)-propionyl]-2,3-dihydro-1H-isoindole-5-carbonitrile

The Boc-bromo isoindoline compound (100 mg, 0.194 mmol) is dissolved in DMF (3 ml), Pd(OAc)₂ (3.484 mg, 0.015 mmol), PPh₃ (8.141 mg, 0.031 mmol) and KCN (12.632 mg, 0.194 mmol) is added and heated at 180° C. in a microwave for 20 min. To the reaction mixture is added brine (50 ml) and extracted with ethyl acetate (50 ml×2). The layers are separated and the organic layer is dried over anhydrous Na₂SO₄, then filtered through Celite and the filtrate is evaporated under reduced pressure. The crude product is purified by flash chromatography on silica gel, eluting with hexane-ethyl acetate using a gradient of 0 to 40% ethyl acetate to give the desired title compound (30%). This nitrile (16 mg, 0.035 mmol) is deprotected as described in Example 19 using TFA in DCM and purified by HPLC to give the desired amino nitrile compound (30%), (m/z 359 [MH+]).

### (R)-2-Amino-1-(5-aminomethyl-1,3-dihydro-isoindol-2-yl)-3-(2,4-dichloro-phenyl)-propan-1-one

2-[(R)-2-Amino-3-(2,4-dichloro-phenyl)-propionyl]-2,3-dihydro-1H-isoindole-5-carbonitrile (500 mg, 1.089 mmol) is suspended in 15:1 dry EtOH/DCM (160 ml) and NiCl₂ (141.156 mg, 1.089 mmol) is added. The reaction mixture is cooled to 0° C. NaBH₄ (123.59 mg, 3.267 mmol) is added slowly and the ice bath is removed. The mixture is allowed to stir at room temperature for 1 h, and then filtered through Celite. The filtrate is concentrated, diluted with brine (50 ml), extracted twice with 50 ml ethyl acetate and the layers are separated. The organic layer is dried over anhydrous Na₂SO₄ and the solvent is evaporated under reduced pressure. The crude product is dissolved in methanol (250 ml), silica-bound tosic acid (excess) is added and the mixture is stirred overnight, and then filtered. The solid support is washed with 2 M ammonia in methanol (50 ml) and the filtrate is concentrated to give (R)-2-Amino-1-(5-aminomethyl-1,3-dihydro-isoindol-2-yl)-3-(2,4-dichloro-phenyl)-propan-1-one (93%), (m/z 363 [MH+]).

### 1-{2-[(R)-2-Amino-3-(2,4-dichloro-phenyl)-propionyl]-2,3-dihydro-1H-isoindol-5-ylmethyl}-3-(4-dimethylamino-phenyl)-urea

(R)-2-Amino-1-(5-aminomethyl-1,3-dihydro-isoindol-2-yl)-3-(2,4-dichloro-phenyl)-propan-1-one (50 mg, 0.108 mmol) and 4-dimethylamino phenyl isocyanate (0.017 ml, 0.108 mmol) are added to THF (3 ml), Et3N (0.023 ml, 0.122 mmol) and stirred at ambient temperature overnight. The crude mixture is dried under reduced pressure and purified by HPLC, yielding 1-{2-[(R)-2-Amino-3-(2,4-dichloro-phenyl)-propionyl]-2,3-dihydro-1H-isoindol-5-ylmethyl}-3-(4-dimethylamino-phenyl)-urea (74%), (m/z 512 [MH+]).

### Example 26

### Preparation of 1-{2-[(R)-2-Amino-3-(2,4-dichloro-phenyl)-propionyl]-2,3-dihydro-1H-isoindol-5-yl}-3-(4-dimethylamino-phenyl)-urea

### 5-nitroisoindoline

5-nitroisoindoline-1,3-dione (5 g, 26.0 mmol) is reduced in the same way as described above in the synthesis 5-bromoisoindoline using BF₃.OEt₂ in the presence of BH₃.THF yielding 5-nitroisoindoline (56%), (m/z 165 [MH+]).

### (R)-tert-butyl 3-(2,4-dichlorophenyl)-1-(5-nitroisoindolin-2-yl)-1-oxopropan-2-yl-carbamate

5-nitroisoindoline (1 g, 6.1 mmol) is coupled to Boc-(D)-2,4-dichlorophenyl alanine (2.04 g, 6.1 mmol) using EDC and HOBt as described in the examples above to yield (R)-tert-butyl 3-(2,4-dichlorophenyl)-1-(5-nitroisoindolin-2-yl)-1-oxopropan-2-yl-carbamate (63%), (m/z 480 [MH+]).

### (R)-tert-butyl 1-(5-aminoisoindolin-2-yl)-3-(2,4-dichlorophenyl)-1-oxopropan-2-ylcarbamate

(R)-tert-butyl 3-(2,4-dichlorophenyl)-1-(5-nitroisoindolin-2-yl)-1-oxopropan-2-yl-carbamate (2.0 g, 4.167 mmol) is dissolved in acetic acid (30 ml), a catalytic amount of Pd/C (10 mol %) is added and the mixture is hydrogenated for 2 h under a balloon of hydrogen gas. The reaction is then filtered through Celite. 10% NaOH (aq) is added to the filtrate until basic. The mixture is then extracted twice with 30 ml ethyl acetate, washed twice with 30 ml of brine and dried over anhydrous Na₂SO₄ and evaporated under reduced pressure. The crude compound is purified with column chromatography using hexane-ethyl acetate (gradient: 0 to 80% ethyl acetate) resulting in (R)-tert-butyl 1-(5-aminoisoindolin-2-yl)-3-(2,4-dichlorophenyl)-1-oxopropan-2-ylcarbamate (92%), (m/z 450 [MH+]).

### 1-{2-[(R)-2-Amino-3-(2,4-dichloro-phenyl)-propionyl]-2,3-dihydro-1H-isoindol-5-yl}-3-(4-dimethylamino-phenyl)-urea

To a solution of (R)-tert-butyl 1-(5-aminoisoindolin-2-yl)-3-(2,4-dichlorophenyl)-1-oxopropan-2-ylcarbamate (50 mg, 0.111 mmol) is added 4-(dimethylamino)-phenyl isocyanate (19.8 mg, 0.122 mmol) and Et₃N (0.023 ml, 0.122 mmol) in THF (3.0 ml). The resulting mixture is stirred at ambient temperature overnight. The solvent is subsequently removed under reduced pressure, to yield the crude product. The mixture is dissolved in DCM (3.0 ml) and TFA (0.5 ml) is added. The mixture is stirred for 1 h at ambient temperature. The solvent is evaporated under reduced pressure and the crude product is purified by HPLC, yielding 1-{2-[(R)-2-Amino-3-(2,4-dichloro-phenyl)-propionyl]-2,3-dihydro-1H-isoindol-5-yl}-3-(4-dimethylamino-phenyl)-urea (74% yield), (m/z 511 [MH+).

### Example 27

### Immunoprecipitation of HDAc from Stable Cell Lines and Elution

Conventional methods are used to express HDAC enzymes and purify them from lysed cells. The following example describes an exemplary procedure, however equivalent procedures are within the scope of the invention.

The cell line used is a derivative of 293 cells overexpressing a fusion of the gene encoding each HDAC protein with a nucleotide sequence encoding the Flag marker. Cells are grown in Optimem, 2% Fetal Calf Serum, Pen/Strep. For enzyme preparation, Lysis buffer (IPLS) is 50 mM Tris-HCl, pH 7.5, 120 mM NaCl, 0.5 mM EDTA and 0.5% Nonidet P-40, to which is added one tablet of Protease inhibitors (Roche 11836170001) per 10 ml buffer. Other buffers are IPHS, which is IPLS containing 1 M NaCl; TBS (Sigma #T5912) dilute 10× stock to 1× with dH₂O; HD buffer: 10 mM Tris pH 8.0 (1M Stock) 10 mM NaCl (5M Stock), 10% glycerol, and for dialysis: 400 μM PMSF is added (for 2 L: use 8 ml 100 mM Stock). Protease inhibitors (Complete mini, Boehringer Mannheim), 1 tablet/10 mL are added to all buffers but not used in buffers for enzyme assays.

Cells are harvested without using trypsin, and most cells are obtained easily in PBS, with gentle striking or agitation of flasks if necessary. More adherent cells are scraped in PBS. Cells are grown in 500 cm² trays, from which about half of the media is aspirated (50 ml total), then cells are scraped in the rest of the media and transferred to a centrifuge tube. Trays are washed with 25 ml cold PBS, scraped again to collect additional cells, and centrifuged at 1500 rpm at 4° C. for 5 min. Cells are washed at least 3 times in PBS to remove growth media, pelleting cells after each wash by centrifugation at 1500 rpm for 5 minutes. After washing, PBS is removed and the resulting cell pellet frozen at −80° C. for storage prior to purification.

For purification, cells are resuspended in lysis buffer, 12 ml of IPLS for cells collected from 10 500 cm trays. Cells are lysed at 4° C. for 3 hrs with rocking, and debris is removed by centrifugation for 20 min at 17,000 rpm in 30 ml centrifuge tubes. If supernatant is not clear afterward, centrifugation of the supernatant is repeated. Protein concentration of the whole cell lysate is determined (generally in the range of about 2-5 mg/ml).

For immunoprecipitation per mg of protein, 15 μL of anti-Flag M2-Agarose Affinity beads (Sigma #A2220) is used. Beads are prepared by washing 3 times with 10× bead volume of PBS and 1 time with IPLS, with centrifugation of the washes at 1500 rpm for 5 min. Whole cell lysate is incubated with the Ab-beads overnight at 4° C. Then beads are centrifuged and washed in 5× volume of the following buffers: three times in IPLS (30 sec at 4° C., spin at 1500 RPM for 5 min); three times in IPHS; and three times in TBS buffer. After each centrifugation, the supernatant is aspirated, leaving the pellet as dry as possible but avoiding sucking up any of the beads.

To elute the enzyme, beads are resuspended in 5× bead volume of TBS with protease inhibitor (Roche 11836170001) 1 tablet/10 mL. Enzyme is eluted with 400 μg/mL Flag peptide (Sigma #F-3290) for 3 hrs at 4° C. on rotator. Then beads are centrifuged, and the supernatant is transferred to a new tube to which is added 1/10 volume of glycerol. The supernatant is transferred to a dialysis cassette (Pierce #66410) using a 3 cc syringe and 18 G needle, and is dialyze sup in 2 L HD buffer for 2 hrs at 4° C. (IL/hour). The resulting purified HDAC is divided into aliquots (300 μL/tube), is snap frozen in dry ice bath, and is stored at −80° C.

### Example 28

### HDAC Fluorescence Assay

For assay of HDAC an assay based on HDAC Fluorescent Activity Assay/Drug Discovery Kit (BioMol #AK500) was used, however any equivalent HDAC assay is within the scope of the invention.

The Fluorescent Assay Buffer (FAB) contains: 25 mM Tris-HCl, pH 8.0, 137 mM NaCl, 2.7 mM KCl and 1 mM MgCl₂. To prepare 20× Developer: 27 mg/mL Trypsin (Sigma #T-8003) is dissolved in Fluorescent Assay Buffer, and is divided into aliquots and stored at −80 C (250 μL/96-well plate). For use, the Developer is diluted to 1× and added 10 μL/mL 0.2 mM TSA

Final assay concentrations are: up to 15 μL HDAC isoform enzyme, 25 μL of substrate (25 uM of rhodamine, 50 uM Fluor de lys substrate, BIOMOL, Plymouth Meeting Pa. available as kit AK-500), and ±10 μL inhibitor diluted in FAB. The final reaction volume of 50 μL is obtained by adding FAB.

All reaction components are prepared in Fluorescent Assay Buffer; enzyme and diluted inhibitors (total volume is 25 μL) are added to clear bottom 96-well ISOPLATE (Wallac #1450-514). The reactions are initiated by adding 25 μL of 100 μM substrate. Negative control wells contain buffer and substrate only or with potent levels of LAQ824 inhibitor.

Enzyme reactions with DMSO are used as positive controls.

The reaction is run for 1-2 hours at 37 C, and reactions are stopped with 50 μL/well of 1× developer containing TSA. Reactions are developed at room temperature for 10 min, and are read with a pre-warmed lamp of Cytofluor Fluorescence Reader. For Fluor de Lys: plates are read at Excitation 360 nm, Emission 460 nm, Gain 65. For Rhodamine: plates are read at Excitation 485 nm, Emission 530 nm, Gain 60.

### Example 29

### p21 Promoter Luciferase Assay Using Stably Transfected p21-Luc in H1299 Cells

### Reagents and General Conditions

The cell lines used are derived from H1299 (p21-luc). The growth media used is RPMI 1640, 10% FBS, 1% Pen/Strep and the selection media added is 500 μg/mL Geneticin (Gibco). The buffer used is 5× cell culture lysis buffer (Promega #E1531), stored at −20 C and the Luciferase-assay reagent (Promega #E1483) is stored at −70 C. The results of the assay are analyzed using Wallac Software.

To assay Luciferase, the cell culture medium is removed after one day of growth and the flasks are washed once with PBS. The cells are trypsinized in 20 mL of media and the trypsin is neutralized. The cells are counted (0.5-1 mL) on a Vi-Cell XR cell viability analyzer.

Cells are then diluted to a concentration of approximately 5000 cells/200 μL, and 190 μL samples are aliquoted into each well of a Costar white 96-well TC treated white bottom plate with lid (Costar #3917). Plates are then incubated overnight at 37 C.

After a further day, a sample of the compounds of the present invention are added to the wells for assay.

After a further day, the cells are lysed and the luciferase activity of the lysed cells is measured. Each well is washed twice with PBS and 20 μL/well of 1× cell culture lysis buffer (dilute 5× to 1× in distilled water) is added to each well. The microtiter plates are then shaken on a microtiter plate shaker for 20 minutes at room temperate at a speed setting of 5-6. After removal from the shaker, 100 μL of Luciferase Reagent is added to each well. Each microtiter plate is then read on Wallac Envision instrument.

### Example 30

### Screening Inhibitory Activity of Compounds

The general procedure to determine the IC50 of compounds using an in vitro cell based assay, cells are seeded into wells of 96-well plates as described above, and are incubated for growth for 24 hours, after which an aliquot of the compound is added at a variety of dilutions to the cells in each well. After further incubation of 72 hours, plates are read.

In general, serial dilutions of each compound are made in cell growth media, and 10 ul samples of dilutions of the compounds are added to the cells, in triplicate (3 rows). Plates are incubated at 37° C. for 72 hours. For determination of activity, CellTiter 96® AQueous One Solution Reagent (Promega), stored frozen, is thawed, protected from light. A sample of 10 μl of CellTiter 96® AQueous One Solution Reagent is added into wells of the 96-well assay plate. Plates are incubated for 3 hours at 37° C. in a humidified, 5% CO₂ atmosphere, and the absorbance at 490 nm is recorded using a 96-well plate reader.

Compounds herein are determined to be active inhibitors of each of the HDAc proteins tested, with some having nanomolar activities. Specific inhibition is observed for each HDAc, for example, a compound inhibits HDAc 1, 2 or 8 preferentially to other HDAc species, however compounds are obtained that inhibit each of the species.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of starting material, synthesis method, or reaction conditions is believed to be a matter of routine for a person of ordinary skill in the art with knowledge of the embodiments described herein. Other aspects, advantages, and modifications considered to be within the scope of the following claims.

1. A compound of formula I, 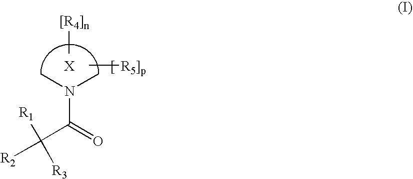
wherein:
R₁ is selected from H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂, and R₃ is a hydrogen;
X is a selected from a C₃-C₆ cycloalkyl, C₃-C₆ cycloalkenyl, aryl, C₃-C₆ heterocycloalkyl, C₃-C₆ heteroaryl, and a polyheterocycle, any of which may be further heterosubstituted, wherein specific examples of polyheterocycles may be selected from 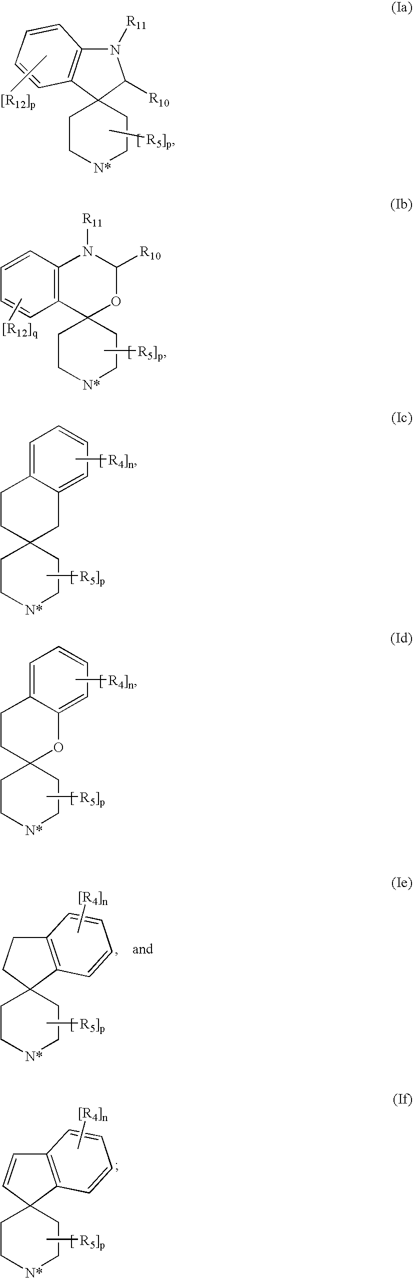
R₄ is present at n occurrences, n is an integer from 0 to 4, and R₄ is the same or different and independently selected from H, lower alkyl, hetero-substituted lower alkyl, alkylaryl, hetero-substituted alkylaryl, lower alkoxy, C₃-C₆ cycloalkyl, aryl, C₃-C₆ heterocycloalkyl, C₃-C₆ heteroaryl, N—(R₁₃)₂, S—R₁₃, O—R₁₃, or a mixed aryl and non-aryl polyheterocycle ring (such as, e.g., benzhydryl or 9H-fluorenyl), any of which may be further substituted by R₈;
R₅ is present at p occurrences, p is an integer from 0 to 4, and R₅ is the same or different and independently selected from H, O, halo, lower alkoxy, and a straight or branched lower alkyl or hetero-substituted lower alkyl;
R₆ is selected from H and a straight or branched lower alkyl;
R₇ is selected from H, C₃-C₁₀ cycloalkyl, C₃-C₁₀ heterocycloalkyl C₃-C₁₀ aryl, C₃-C₁₀ heteroaryl, oxyaryl, arylalkone, and cycloalkylaryl, any of which may be further substituted by R₈;
R₈ is selected from one or more of H, halo, lower alkyl, hetero-substituted lower alkyl, lower alkenyl, lower alkoxy, C₃-C₁₀ cycloalkyl C₃-C₁₀ heterocycloalkyl, C₃-C₁₀ aryl, C₃-C₁₀ heteroaryl, arylalkyl, heteroarylalkyl, acid alkylester, alkone, alkoxy; any of which may be further substituted by R₉; and
R₉ is selected from one or more of H, halo, COOH, lower alkyl, hetero-substituted lower alkyl, aryl, and lower alkoxy;
R₁₀ and R₁₁ are selected from H, O, halo, lower alkyl, hetero-substituted lower alkyl, and lower alkoxy; and
R₁₂ is present at q occurrences wherein q is an integer from 0 to 4, and R₁₂ is the same or different and independently selected from are selected from H, O, halo, lower alkyl, hetero-substituted lower alkyl, and lower alkoxy;
R₁₃ is selected from one or more of H, lower alkyl, hetero-substituted lower alkyl lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted, by R₈;
or a pharmaceutically acceptable salt thereof.

2. A compound of formula II 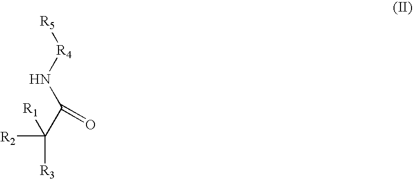
wherein:
R₁ can be H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
R₄ is selected from C₃-C₆ cycloalkyl, aryl, C₃-C₆ heterocycloalkyl, C₃-C₆ heteroaryl, or a mixed aryl and non-aryl polyheterocycle ring, any of which may be further substituted by R₇;
R₅ is present at p occurrences, p is an integer from 0 to 3, and R₅ is the same or different and independently selected from H, O, halo, lower alkoxy, and a straight or branched lower alkyl or hetero-substituted lower alkyl;
R₆ is H or a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₈ is selected from H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy, N—(R₁₃)₂; S—R₁₃, O—R₁₃; any of which may be further substituted by R₉;
R₉ is selected from H, halo, lower alkyl, hetero-substituted lower alkyl, aryl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
or a pharmaceutically acceptable salt of any of these compounds.
or a pharmaceutically acceptable salt thereof.

3. The compound of claim 1 selected from subformula III through subformula V 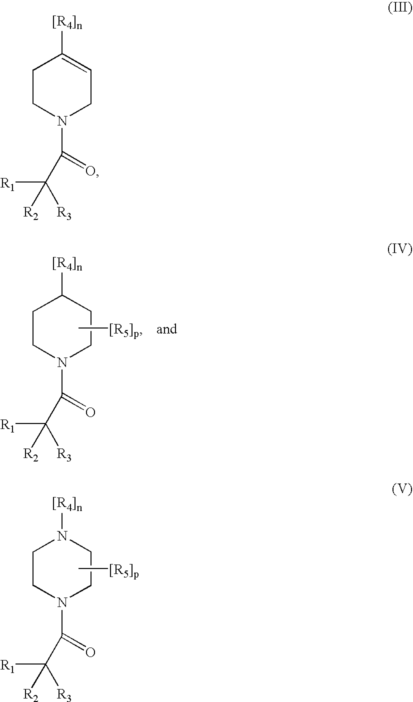
wherein:
R₁ is selected from H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
R₄ is present at n occurrences wherein n is an integer from 0 to 4, and R₄ is the same or different and independently selected from H, lower alkyl, hetero-substituted lower alkyl, lower alkoxy, alkylaryl, hetero-substituted alkylaryl, C₃-C₆ cycloalkyl, aryl, C₃-C₆ heterocycloalkyl, C₃-C₆ heteroaryl, N—(R₁₃)₂, S—R₁₃, O—R₁₃, or a mixed aryl and non-aryl polyheterocycle ring (such as, e.g., benzhydryl or 9H-fluorenyl), any of which may be further substituted by R₈;
R₅ is present at p occurrences wherein p is an integer from 0 to 4, and R₅ is the same or different and independently selected from H, O, halo, lower alkoxy, and a straight or branched lower alkyl or hetero-substituted lower alkyl;
R₆ is selected from H and a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₆ is selected from one or more of H, O, halo, lower alkyl, hetero-substituted lower alkyl lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy; any of which may be further substituted by R₉;
R₉ is selected from one or more of H, halo, COOH, lower alkyl, hetero-substituted lower alkyl, aryl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
or a pharmaceutically acceptable salt thereof.

4. The compound of claim 1 selected from formula Ia through formula Ij: 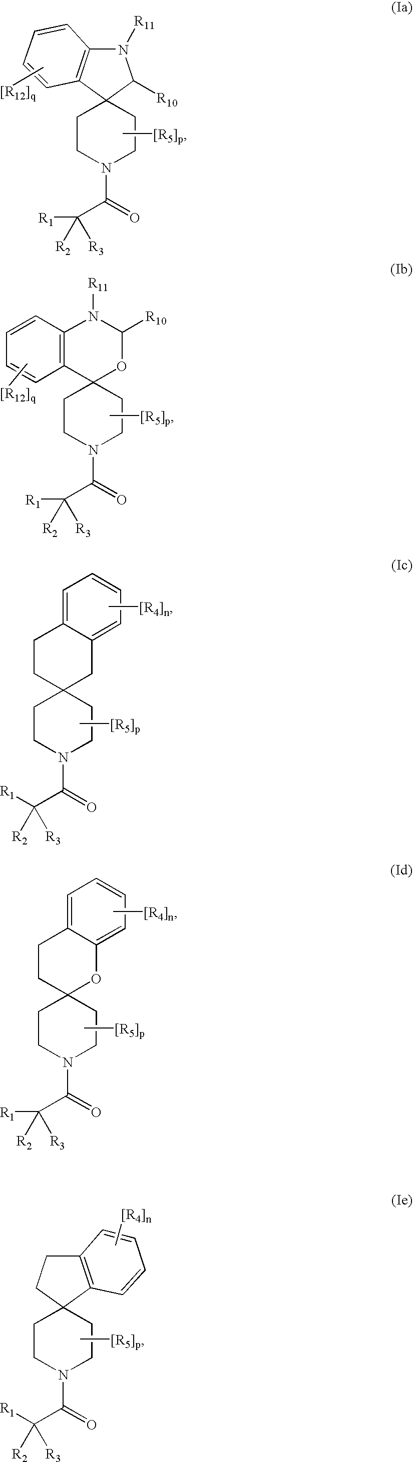 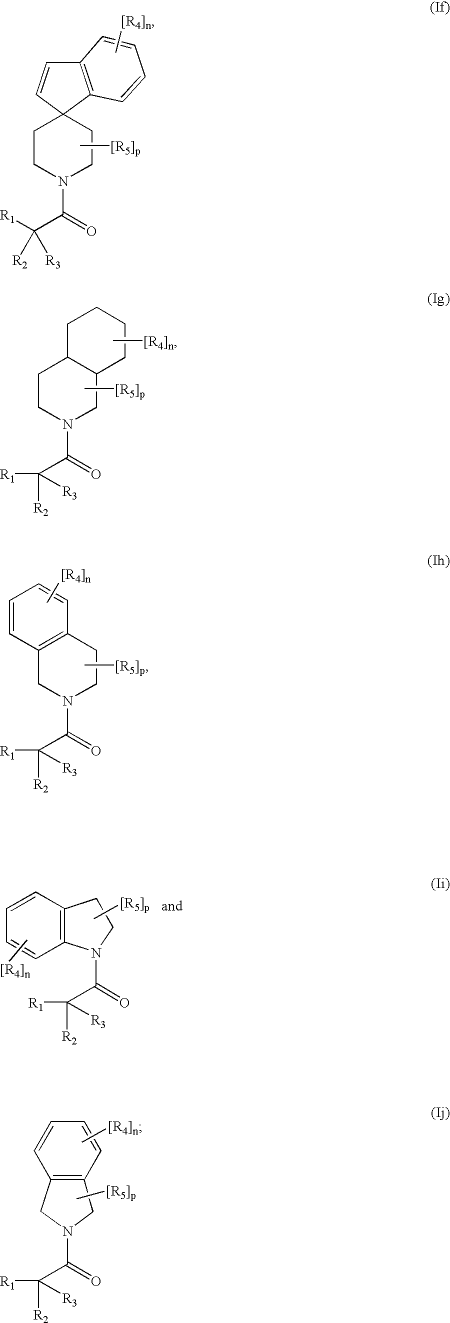
wherein:
R₁ is selected from H, NH₂, NHR₆, SR₆, SOR₆, O, and OR₆;
R₂ and R₃ are independently selected from H, a straight or branched chain C₁-C₆ alkyl, a straight or branched chain C₁-C₆R₇ alkyl or alkenyl, any of which may optionally be heterosubstituted, and wherein at least one of R₂ and R₃ is a hydrogen;
R₄ is present at n occurrences wherein n is an integer from 0 to 4, and R₄ is the same or different and independently selected from H, lower alkyl, hetero-substituted lower alkyl, alkylaryl, hetero-substituted alkylaryl, lower alkoxy, C₃-C₆ cycloalkyl, aryl, C₃-C₆ heterocycloalkyl, C₃-C₆ heteroaryl, N—(R₁₃)₂, S—R₁₃, O—R₁₃, or a mixed aryl and non-aryl polyheterocycle ring, (such as, e.g., benzhydryl or 9H-fluorenyl), any of which may be further substituted by R₈;
R₅ is present at p occurrences wherein p is an integer from 0 to 4, and R₅ is the same or different and independently selected from H, O, halo, lower alkoxy, and a straight or branched lower alkyl or hetero-substituted lower alkyl;
R₆ is selected from H and a straight or branched lower alkyl;
R₇ is selected from H, C₃-C₁₀ cycloalkyl, C₃-C₁₀ heterocycloalkyl, C₃-C₁₀ aryl, C₃-C₁₀ heteroaryl, oxyaryl, arylalkone, and cycloalkylaryl, any of which may be further substituted by R₈;
R₈ is selected from one or more of H, halo, lower alkyl, hetero-substituted lower alkyl, lower alkenyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl, acid alkylester, alkone, alkoxy; any of which may be further substituted by R₉;
R₉ is selected from one or more of H, halo, COOH, lower alkyl, hetero-substituted lower alkyl, aryl, and lower alkoxy;
R₁₀ and R₁₁ are selected from H, O, halo, lower alkyl, hetero-substituted lower alkyl, and lower alkoxy;
R₁₂ is present at q occurrences wherein q is an integer from 0 to 4, and R₁₂ is the same or different and independently selected from are selected from H, O, halo, lower alkyl, hetero-substituted lower alkyl, and lower alkoxy; and
R₁₃ is selected from one or more of H, lower alkyl, hetero-substituted lower alkyl, lower alkoxy, C₃-C₁₀ cycloalkyl, C₃-C₁₀ heterocycloalkyl, C₃-C₁₀ aryl, C₃-C₁₀ heteroaryl, arylalkyl, heteroarylalkyl; any of which may be further substituted by R₈;
or a pharmaceutically acceptable salt thereof.

5. The compound according to claim 1, wherein at least one of R₁, R₂, R₃ is selected from hydrogen.

6. The compound according to claim 1, wherein at least one of R₁, R₂, and R₃ is selected from the group of NHR₆ or NH₂.

7. The compound according to according to claim 1, wherein R₁ is H, and R₂ is NH₂.

8. A compound selected from the group consisting of:
4-Biphenyl-3-yl-1,2,3,6-tetrahydro-pyridine;
[2-(4-Benzofuran-2-yl-3,6-dihydro-2H-pyridin-1-yl)-2-oxo-ethyl]-carbamic acid tert-butyl ester;
[2-(4-Biphenyl-3-yl-3,6-dihydro-2H-pyridin-1-yl)-2-oxo-ethyl]-carbamic acid tert-butyl ester;
2-Amino-1-(4-benzofuran-2-yl-3,6-dihydro-2H-pyridin-1-yl)ethanone;
2-Amino-1-(4-biphenyl-3-yl-3,6-dihydro-2H-pyridin-1-yl)-ethanone;
2-Amino-1-(4-biphenyl-3-yl-piperidin-1-yl)-ethanone;
2-Amino-1-[4-(2-morpholin-4-ylmethyl-phenyl)-3,6-dihydro-2H-pyridin-1-yl]-ethanone;
2-Amino-1-(4-benzhydryl-piperazin-1-yl)ethanone;
N-(2-Acetyl-2,3-dihydro-1H-isoindol-5-yl)-benzamide;
2-Amino-1-[4-(5-chloro-2-methyl-phenyl)-piperazin-1-yl]-5-phenyl-pent-4-en-1-one;
2-Amino-1-(4-benzhydryl-piperazin-1-yl)-5-phenyl-pent-4-en-1-one;
N-{2-[2-Amino-3-(2,4-dichloro-phenyl)-propionyl]-2,3-dihydro-1H-isoindol-5-yl}-5-fluoro-2-trifluoromethyl-benzamide;
N-{2-[2-Amino-3-(2,4-dichloro-phenyl)-propionyl]-2,3-dihydro-1H-isoindol-5-yl}-5-chloro-2-trifluoromethyl-benzamide;
2-Amino-3-(4-chloro-phenyl)-1-[4-(3-chloro-phenyl)-piperidin-1-yl]-propan-1-one;
2-Amino-3-(4-chloro-phenyl)-1-[4-(3-chloro-phenyl)-3,6-dihydro-2H-pyridin-1-yl]-propan-1-one;
2-Amino-1-(4-biphenyl-3-yl-3,6-dihydro-2H-pyridin-1-yl)-3-(4-chloro-phenyl)-propan-1-one;
2-Amino-1-[4-(5-chloro-2-methyl-phenyl)-3,6-dihydro-2H-pyridin-1-yl]-3-(4-chloro-phenyl)-propan-1-one;
{1-(4-Chloro-benzyl)-2-[4-(5-chloro-2-methyl-phenyl)-3,6-dihydro-2H-pyridin-1-yl]-2-oxo-ethyl}-carbamic acid tert-butyl ester;
[2-(4-Benzofuran-2-yl-3,6-dihydro-2H-pyridin-1-yl)-1-(4-chloro-benzyl)-2-oxo-ethyl]-carbamic acid tert-butyl ester;
[2-(4-Biphenyl-3-yl-piperidin-1-yl)-1-(4-chloro-benzyl)-2-oxo-ethyl]-carbamic acid tert-butyl ester;
2-Amino-1-(4-biphenyl-3-yl-piperidin-1-yl)-3-(4-chloro-phenyl)-propan-1-one;
2-Amino-1-(4-benzofuran-2-yl-3,6-dihydro-2H-pyridin-1-yl)-3-(4-chloro-phenyl)-propan-1-one;
2-Amino-3-(4-benzyloxy-phenyl)-1-(4-biphenyl-3-yl-piperidin-1-yl)-propan-1-one;
2-Amino-3-(4-chloro-phenyl)-1-[4-(2-morpholin-4-ylmethyl-phenyl)-3,6-dihydro-2H-pyridin-1-yl]-propan-1-one;
2-Amino-1-(4-biphenyl-3-yl-piperidin-1-yl)-3-(3,4-dichloro-phenyl)-propan-1-one;
2-Amino-1-(4-benzhydryl-piperazin-1-yl)-3-(4-chloro-phenyl)-propan-1-one;
2-Amino-1-(4-biphenyl-3-yl-piperidin-1-yl)-3-pyridin-4-yl-propan-1-one;
2-Amino-1-(4-biphenyl-3-yl-piperidin-1-yl)-3-(4-hydroxy-phenyl)-propan-1-one;
1-(4-Biphenyl-3-yl-piperidin-1-yl)-3-(4-chloro-phenyl)-2-methylamino-propan-1-one;
1-(4-Biphenyl-3-yl-3,6-dihydro-2H-pyridin-1-yl)-3-(4-chloro-phenyl)-2-methylamino-propan-1-one;
2-Amino-1-(4-biphenyl-3-yl-piperidin-1-yl)-3-(2,4-dichloro-phenyl)-propan-1-one;
2-Amino-1-(4-biphenyl-3-yl-piperidin-1-yl)-3-p-tolyl-propan-1-one;
2-Amino-3-(4-chloro-phenyl)-1-[4-(4-fluoro-phenyl)-piperazin-1-yl]-propan-1-one
2-Amino-1-(4-biphenyl-3-yl-piperidin-1-yl)-2-phenyl-ethanone;
2-Amino-1-(4-benzhydryl-piperazin-1-yl)-2-phenyl-ethanone;
2-Amino-3-(3,4-dichloro-phenyl)-1-[4-(4-fluoro-phenyl)-piperazin-1-yl]-propan-1-one;
2-Amino-1-(4-benzhydryl-piperazin-1-yl)-3-naphthalen-1-yl-propan-1-one;
2-Amino-1-(4-benzhydryl-piperazin-1-yl)-3-phenyl-propan-1-one;
4-[2-Amino-3-(4-benzhydryl-piperazin-1-yl)-3-oxo-propyl]-benzonitrile;
2-Amino-1-(4-biphenyl-3-yl-piperidin-1-yl)-3-phenyl-propan-1-one;
2-Amino-3-(4-chloro-phenyl)-1-(4-naphthalen-1-yl-3,6-dihydro-2H-pyridin-1-yl)-propan-1-one;
2-Amino 1-(4-benzhydryl-piperazin-1-yl)-3-biphenyl-4-yl-propan-1-one;
2-Amino-1-(4-benzofuran-2-yl-3,6-dihydro-2H-pyridin-1-yl)-3-(2,4-dichloro-phenyl)-propan-1-one;
2-Amino-3-(2,4-dichloro-phenyl)-1-[4-(3-fluoro-phenyl)-3,6-dihydro-2H-pyridin-1-yl]-propan-1-one;
2-Amino-1-(4-benzhydryl-piperazin-1-yl)-3-(2-chloro-phenyl)-propan-1-one;
2-[2-Amino-3-(4-benzhydryl-piperazin-1-yl)-3-oxo-propyl]-benzonitrile;
2-Amino-1-[4-(3-fluoro-benzyl)-piperazin-1-yl]-3-phenyl-propane-1-one;
2-Amino-1-[4-(4-fluoro-phenyl)-piperazin-1-yl]-3-p-tolyl-propan-1-one;
2-Amino-3-(4-benzyloxy-phenyl)-1-[4-(4-fluoro-phenyl)-piperazin-1-yl]-propan-1-one;
4-{2-Amino-3-[4-(4-fluoro-phenyl)-piperazin-1-yl]-3-oxo-propyl}-benzonitrile;
2-Amino-3-biphenyl-4-yl-1-[4-(4-fluoro-phenyl)-piperazin-1-yl]-propan-1-one;
2-Amino-1-[4-(4-fluoro-phenyl)-piperazin-1-yl]-3-phenyl-propan-1-one;
2-Amino-1-(4-benzhydryl-piperazin-1-yl)-3-(2,4-dichloro-phenyl)-propan-1-one;
2-Amino-3-(2,4-dichlorophenyl)-1-[4-(3-fluoro-benzyl)-piperazin-1-yl]-propan-1-one;
2-Amino-3-(2-chloro-phenyl)-1-[4-(3-fluoro-benzyl)-piperazin-1-yl]-propan-1-one;
2-Amino-3-(4-chloro-phenyl)-1-[4-(3-fluoro-benzyl)-piperazin-1-yl]-propan-1-one;
N-(3-{1-[2-Amino-3-(2,4-dichloro-phenyl)-propionyl]-piperidin-4-yl}-phenyl)acetamide;
N-(3-{1-[2-Amino-3-(2,4-dichloro-phenyl)-propionyl]-piperidin-4-yl}-phenyl)-benzamide;
2-Amino-1-(4-benzhydryl-piperazin-1-yl)-3-thiophen-3-yl-propan-1-one;
2-Amino-1-(4-benzhydryl-piperazin-1-yl)-3-thiophen-2-yl-propan-1-one;
2-Amino-1-[4-(5-chloro-2-methyl-phenyl)-piperazin-1-yl]-3-thiophen-2-yl-propan-1-one;
2-Amino-1-[4-(5-chloro-2-methyl-phenyl)-piperazin-1-yl]-3-phenyl-propan-1-one;
2-Amino-1-[4-(5-chloro-2-methyl-phenyl)-piperazin-1-yl]-3-(4-chloro-phenyl)-propan-1-one;
2-Amino-1-[4-(5-chloro-2-methyl-phenyl)-piperazin-1-yl]-3-(2,4-dichloro phenyl)-propan-1-one;
2-Amino-3-biphenyl-4-yl-1-[4-(5-chloro-2-methyl-phenyl)-piperazin-1-yl]-propan-1-one;
2-Amino-1-[4-(5-chloro-2-methyl-phenyl)-piperazin-1-yl]-3-(4-trifluoromethyl-phenyl)-propan-1-one;
N-(3-{1-[2-Amino-3-(4-chloro-phenyl)-propionyl]-piperidin-4-yl}-phenyl)-benzamide;
2-Amino-1-(4-biphenyl-3-yl-piperidin-1-yl)-3-thiophen-2-yl-propan-1-one;
2-Amino-1-(4-biphenyl-3-yl-piperidin-1-yl)-3-thiophen-3-yl-propan-1-one;
2-Amino-1-[4-(5-chloro-2-methyl-phenyl)-piperazin-1-yl]-3-thiophen-3-yl-propan-1-one;
[2-[4-(5-Chloro-2-methyl-phenyl)-piperazin-1-yl]-1-(2,4-dichloro-benzyl)-2-oxo-ethyl]-carbamic acid tert-butyl ester;
2-Amino-1-(4-benzofuran-2-yl-piperidin-1-yl)-3-(2,4-dichlorophenyl)-propan-1-one;
Thioacetic acid -[2-(4-benzhydryl-piperazin-1-yl)-1-benzyl-2-oxo-ethyl]ester;
2-Amino-3-(5-bromo-thiophen-2-yl)-1-(4-naphthalen-1-yl-piperidin-1-yl)-propan-1-one;
2-Amino-3-(5-bromo-thiophen-2-yl)-1-[4-(5-chloro-2-methyl-phenyl)-piperazin-1-yl]-propan-1-one;
2-{2-Amino-3-[4-(5-chloro-2-methyl-phenyl)-piperazin-1-yl]-3-oxo-propyl}-benzonitrile;
2-Amino-3-(2-chloro-phenyl)-1-(4-naphthalen-1-yl-piperidin-1-yl)-propan-1-one;
2-Amino-1-(4-benzoyl-piperazin-1-yl)-3-(2-chloro-phenyl)-propan-1-one;
2-Amino-3-(2,4-dichloro-phenyl)-1-(4-naphthalen-1-yl-piperidin-1-yl)-propan-1-one;
2-Amino-3-(4-chloro-phenyl)-1-(4-naphthalen-1-yl-piperidin-1-yl)-propan-1-one;
2-Amino-1-[4-(5-chloro-2-methyl-phenyl)-piperazin-1-yl]-3-(2-chloro-phenyl)-propan-1-one;
2-Amino-3-(2,4-dichloro-phenyl)-1-[4-(4-fluoro-phenyl)-piperazin-1-yl]-propan-1-one;
2-Amino-3-(2-chlorophenyl)-1-[4-(4-fluoro-phenyl)-piperazin-1-yl]-propan-1-one;
2-Amino-3-(5-bromo-thiophen-2-yl)-1-[4-(4-fluoro-phenyl)-piperazin-1-yl]-propan-1-one;
2-Amino-1-(4-benzhydryl-piperazin-1-yl)-3-(5-bromo-thiophen-2-yl)-propan-1-one;
2-Amino-1-(4-benzoyl-piperazin-1-yl)-3-(2,4-dichloro-phenyl)-propan-1-one;
2-Amino-1-[4-(5-chloro-2-methyl-phenyl)-piperazin-1-yl]-3-furan-2-yl-propan-1-one;
2-Amino-1-(4-benzhydryl-piperazin-1-yl)-3-thiazol-5-yl-propan-1-one;
2-Amino-1-(4-biphenyl-3-yl-piperidin-1-yl)-3-furan-2-yl-propan-1-one;
2-Amino-1-(4-biphenyl-3-yl-piperidin-1-yl)-5-phenyl-pent-4-en-1-one;
2-Amino-1-(4-biphenyl-3-yl-piperidin-1-yl)-3-(5-bromo-thiophen-2-yl)-propan-1-one;
2-Amino-1-(4-benzhydryl-piperazin-1-yl)-3-(3-chloro-phenyl)-propan-1-one;
2-Amino-1-(4-benzhydryl-piperazin-1-yl)-3-(3-methyl-3H-imidazol-4-yl)-propan-1-one;
2-Amino-1-(4-benzhydryl-piperazin-1-yl)-3-(4-fluoro-phenyl)-propan-1-one;
2-Amino-1-(4-benzhydryl-piperazin-1-yl)-(2-fluoro-phenyl)-propan-1-one;
2-Amino-1-(4-benzhydryl-piperazin-1-yl)-3-o-tolyl-propan-1-one;
2-Amino-1-[4-(5-chloro-2-methyl-phenyl)-piperazin-1-yl]-3-(3-trifluoromethyl-phenyl)-propan-1-one;
2-Amino-1-[4-(5-chloro-2-methyl-phenyl)-piperazin-1-yl]-3-(4-fluoro-phenyl)-propan-1-one;
2-Amino-1-[4-(5-chloro-2-methyl-phenyl)-piperazin-1-yl]-3-(3-chloro-phenyl)-propan-1-one;
2-Amino-1-[4-(5-chloro-2-methyl-phenyl)-piperazin-1-yl]-3-o-tolyl-propan-1-one;
2-Amino-1-[4-(5-chloro-2-methyl-phenyl)-piperazin-1-yl]-3-m-tolyl-propan-1-one;
2-Amino-1-[4-(5-chloro-2-methyl-phenyl)-piperazin-1-yl]-3-thiazol-4-yl-propan-1-one;
2-Amino 1-[4-(5-chloro-2-methyl-phenyl)-piperazin-1-yl]-3-(2-trifluoromethyl-phenyl)-propan-1-one;
2-Amino-1-[4-(5-chloro-2-methyl-phenyl)-piperazin-1-yl]-3-(3-methyl-3H-imidazol-4-yl)-propan-1-one;
2-Amino-1-(4-benzhydryl-piperazin-1-yl)-3-(2-trifluoromethyl-phenyl)-propan-1-one;
2-Amino-1-(4-benzhydryl-piperazin-1-yl)-3-m-tolyl-propan-1-one;
2-Amino-1-(4-benzhydryl-piperazin-1-yl)-3-(3-trifluoromethyl-phenyl)-propan-1-one;
2-Amino-1-(4-benzoyl-piperazin-1-yl)-3-thiazol-4-yl-propan-1-one;
2-Amino-1-(4-benzhydryl-piperazin-1-yl)-3-(1-methyl-1H-imidazol-4-yl)-propan-1-one;
2-Amino-1-(4-benzhydryl-piperazin-1-yl)-3-(1H-imidazol-4-yl)-propan-1-one;
2-Amino-1-(4-benzoyl-piperazin-1-yl)-3-(4-fluoro-phenyl)-propan-1-one;
2-Amino-1-(4-benzoyl-piperazin-1-yl)-3-(2-fluoro-phenyl)-propan-1-one;
2-Amino-1-(4-benzoyl-piperazin-1-yl)-3-(3-trifluoromethyl-phenyl)-propan-1-one;
2-Amino-1-(4-benzoyl-piperazin-1-yl)-3-(3-chloro-phenyl)-propan-1-one;
2-Amino-1-(4-benzoyl-piperazin-1-yl)-3-(2-trifluoromethyl-phenyl)-propan-1-one;
2-Amino-1-(4-benzoyl-piperazin-1-yl)-3-m-tolyl-propan-1-one;
2-Amino-1-(4-benzoyl-piperazin-1-yl)-3-o-tolyl-propan-1-one;
2-Amino-1-(4-benzoyl-piperazin-1-yl)-3-(3-methyl-3H-imidazol-4-yl)-propan-1-one;
2-Amino-1-(4-benzhydryl-piperazin-1-yl)-3-[5-(4-chloro-phenyl)-thiophen-2-yl]-propan-1-one;
2-Amino-1-(4-benzhydryl-piperazin-1-yl)-3-[5-(3-chloro-phenyl)-propan-2-yl]-propan-1-one;
2-Amino-1-(4-benzhydryl-piperazin-1-yl)-3-[5-(2-chloro-phenyl)-thiophen-2-yl]-propan-1-one;
2-Amino-1-(4-benzoyl-piperazin-1-yl)-3-(1H-imidazo-4-yl)-propan-1-one;
2-Amino-1-(4-benzoyl-piperazin-1-yl)-3-thiophen-3-yl-propan-1-one;
2-Amino-1-(4-benzoyl-piperazin-1-yl)-3-thiophen-2-yl-propan-1-one;
2-Amino-1-(4-benzoyl-piperazin-1-yl)-3-(5-bromo-thiophen-2-yl)-propan-1-one;
2-Amino-1-(4-benzoyl-piperazin-1-yl)-3-furan-2-yl-propan-1-one;
2-Amino-1-(4-benzhydryl-piperazin-1-yl)-3-[5-(2,4-dichloro-phenyl)-thiophen-2-yl]-propan-1-one;
2-Amino-1-[4-(5-chloro-2-methoxy-phenyl)-piperazin-1-yl]-3-(2-dichloro-phenyl)-propan-1-one;
2-Amino-1-[4-(5-chloro-2-methoxy-phenyl)-piperazin-1-yl]-3-(2-chloro-phenyl)-propan-1-one;
2-Amino-1-[4-(3-chloro-phenyl)-piperazin-1-yl]-3-(2,4-dichloro-phenyl)-propan-1-one;
2-Amino-3-(2-chloro-phenyl)-1-[4-(3-chloro-phenyl)-piperazin-1-yl]-propan-1-one;
2-Amino-3-(2,4-dichloro-phenyl)-1-{4-[3-(4-methyl-piperazin-1-ylmethyl)-phenyl]-piperidin-1-yl}-propan-1-one;
1-[4-(5-Chloro-2-methyl-phenyl)-piperazin-1-yl]-3-(2,4-dichloro-phenyl)-2-mercapto-propan-1-one;
2-Amino 1-[4-(3-chloro-phenyl)-piperazin-1-yl]-3-m-tolyl-propan-1-one;
2-Amino-3-(3-chloro-phenyl)-1-[4-(3-chlorophenyl)-piperazin-1-yl]-propan-1-one;
2-Amino-1-[4-(5-chloro-2-methoxy-phenyl)-piperazin-1-yl]-3-(3-chloro-phenyl)-propan-1-one;
2-Amino-3-[4-(3-fluoro-benzyl)-piperazin-1-yl]-3-(3-trifluoromethyl-phenyl)-propan-1-one;
2-Amino-1-[4-(3-fluoro-benzyl)-piperazin-1-yl]-3-(2-trifluoromethyl-phenyl)-propan-1-one;
2-Amino-3-(3-chloro-phenyl)-1-[4-(3-fluoro-benzyl)-piperazin-1-yl]-propan-1-one;
2-Amino-3-(2,4-dichloro-phenyl)-1-piperazin-1-yl-propan-1-one;
2-Amino-3-(2,4-dichloro-phenyl)-1-morpholin-4-yl-propan-1-one;
2-Amino-3-(2,4-chlorophenyl)-1-(4-methyl-piperazin-1-yl)-propan-1-one;
2-Amino-3-(2,4-dichloro-phenyl)-1-[4-(5-fluoro-2-trifluoromethyl-benzoyl)-piperazin-1-yl]-propan-1-one;
2-Amino-3-(2-chloro-phenyl)-1-[4-(5-fluoro-2-trifluoromethyl-benzyl)-piperazin-1-yl]-propan-1-one;
2-Amino-1-[4-(5-fluoro-2-trifluoromethyl-benzoyl)-piperazin-1-yl]-3-(2-trifluoromethyl-phenyl)-propan-1-one;
2-Amino-1-[4-(5-fluoro-2-trifluoromethyl-benzoyl)-piperazin-1-yl]-3-(2-trifluoromethyl-phenyl)-propan-1-one;
2-Amino-1-[4-(5-fluoro-2-trifluoromethyl-benzoyl)-piperazin-1-yl]-3-thiophen-2-yl-propan-1-one;
2-Amino-3-(5-bromo-thiophen-2-yl)-1-[4-(5-fluoro-2-trifluoromethyl-benzoyl)-piperazin-1-yl]-propan-1-one;
1-(4-Benzhydryl-piperazin-1-yl)-3-(2,4-dichloro-phenyl)-2-mercapto-propan-1-one;
2-Amino-3-(2,4-dichloro-phenyl)-1-(4-indan-2-yl-piperazin-1-yl)-propan-1-one;
2-Amino-3-(2,4-dichloro-phenyl)-1-(4-pyridin-2-ylmethyl-piperazin-1-yl)-propan-1-one;
2-Amino-1-(4-indan-2-yl-piperazin-1-yl)-3-thiophen-2-yl-propan-1-one;
2-Amino-3-(2,4-dichloro-phenyl)-1-[4-(3-thiophen-2-yl-benzyl)-piperazin-1-yl]-propan-1-one;
2-Amino-1-[4-(3′-chloro-biphenyl-3-ylmethyl)-piperazin-1-yl]-3-(2,4-dichloro-phenyl)-propan-1-one;
2-Amino-3-(2,4-dichloro-phenyl)-1-(4-pyridin-3-yl-methyl-piperazin-1-yl)-propan-1-one;
2-Amino-3-(2,4-dichloro-phenyl)-1-(4-pyridin-4-ylmethyl-piperazin-1-yl)-propan-1-one;
2-Amino-3-(2,4-dichloro-phenyl)-1-[4-(2,5-difluoro-benzyl)-piperazin-1-yl]-propan-1-one;
2-Amino-1-{4-[3-(3,5-dichloro-phenoxy)-benzyl]-piperazin-1-yl}-3-(2,4-dichloro-phenyl)-propan-1-one;
2-Amino-3-(2,4-dichloro-phenyl)-1-[4-(3-methyl-benzyl)-piperazin-1-yl]-propan-1-one;
2-Amino-3-(2,4-dichloro-phenyl)-1-[4-(4-fluoro-benzyl)-piperazin-1-yl]-propan-1-one;
2-Amino-3-(2,4-dichlorophenyl)-1-[4-(pyridine-2-carbonyl)-piperazin-1-yl]-propan-1-one;
2-Amino-3-(2-chloro-phenyl)-1-[4-(pyridine-2-carbonyl)-piperazin-1-yl]-propan-1-one;
2-Amino-3-(3-chloro-phenyl)-1-[4-(pyridine-2-carbonyl)-piperazin-1-yl]-propan-1-one;
2-Amino-3-(5-bromo-thiophen-2-yl)-1-[4-pyridine-2-carbonyl)-piperazin-1-yl]-propan-1-one;
2-Amino-3-(2,4-dichloro-phenyl)-1-[4-(4-methoxy-benzyl)-piperazin-1-yl]-propan-1-one;
1-(4-Benzhydryl-piperazin-1-yl)-3-(3-chloro-phenyl)-2-mercapto-propan-1-one;
1-[4-(5-Chloro-2-methyl-phenyl)-piperazin-1-yl]-3-(3-chloro-phenyl)-2-mercapto-propan-1-one;
3-(3-Chloro-phenyl)-1-[4-(5-fluoro-2-trifluoromethyl-benzoyl)-piperazin-1-yl]-2-mercapto-propan-1-one;
2-Amino-3-(2,4-dichloro-phenyl)-1-[4-(4-methyl-benzyl)-piperazin-1-yl]-propan-1-one;
2-Amino-1-(4-cyclohexanecarbonyl-piperazin-1-yl)-3-(2,4-dichloro-phenyl)-propan-1-one;
2-Amino-1-{4-[bis-(4-fluoro-phenyl)-methyl]-piperazin-1-yl}-3-(2,4-dichloro-phenyl)-propan-1-one;
2-Amino-3-(2,4-dichloro-phenyl)-1-[4-(4-pyrimidin-5-yl-benzyl)-piperazin 1-yl]-propan-1-one;
2-Amino-3-(2,4-dichloro-phenyl)-1-[4-(4-pyridin-4-yl-benzyl)-piperazin-1-yl]-propan-1-one;
2-Amino-3-(2,4-dichloro-phenyl)-1-[4-(3-fluoro-pyridin-4-ylmethyl)-piperazin-1-yl]-propan-1-one;
2-Amino-1-[4-(3,4-dichloro-benzyl)-piperazin-1-yl]-3-(2,4-dichloro-phenyl)-propan-1-one;
2-Amino-1-[4-(pyridine-2-carbonyl)-piperazin-1-yl]-3-(2-trifluoromethyl-phenyl)-propan-1-one;
2-Amino-3-(2-fluoro-phenyl)-1-[4-pyridine-2-carbonyl)-piperazin-1-yl]-propan-1-one;
2-Amino-3-(5-bromo-thiophen-2-yl)-1-(4-indan-2-yl-piperazin-1-yl)-propan-1-one;
2-Amino-1-[4-(6-chloro-pyridin-2-yl)-piperazin-1-yl]-3-(2,4-dichloro-phenyl)-propan-1-one;
2-Amino-1-[4-(1-chloro-2-trifluoromethyl-benzoyl)-piperazin-1-yl]-3-(2,4-dichloro-phenyl)-propan-1-one;
2-Amino-3-(2-chloro-phenyl)-1-[4-(5-chloro-2-trifluoromethyl-benzoyl)-piperazin-1-yl]-propan-1-one;
2-Amino-3-(3-chloro-phenyl)-1-[4-(5-chloro-2-trifluoromethyl-benzoyl)-piperazin-1-yl]-propan-1-one;
2-Amino-1-[4-(5-chloro-2-trifluoromethyl-benzoyl)-piperazin-1-yl]-3-(2-trifluoromethyl-phenyl)-propan-1-one;
2-Amino-1-[4-(5-chloro-2-trifluoromethyl-benzoyl)-piperazin-1-yl]-3-thiophen-2-yl-propan-1-one;
2-Amino-3-(5-bromo-thiophen-2-yl)-1-[4-(5-chloro-2-trifluoromethyl-benzoyl)-piperazin-1-yl]-propan-1-one;
2-Amino-3-(5-bromo-thiophen-2-yl)-1-[4-(5-chloro-2-methoxy-phenyl)-piperazin-1-yl]-propan-1-one;
2-Amino-3-(2,4-dichloro-phenyl)-1-[4-(2,5-difluoro-benzoyl)-piperazin-1-yl]-propan-1-one;
2-Amino-3-(2-chloro-phenyl)-1-[4-(2,5-difluoro-benzoyl)-piperazin-1-yl]-propan-1-one
2-Amino-3-(3-chloro-phenyl)-1-[4-(2,5-difluoro-benzoyl)-piperazin-1-yl]-propan-1-one;
2-Amino-1-[4-(2,5-difluoro-benzoyl)-piperazin-1-yl]-3-(2-trifluoromethyl-phenyl)-propan-1-one;
2-Amino-1-[4-(2,5-difluoro-benzoyl)-piperazin-1-yl]-3-thiophen-2-yl-propan-1-one;
2-Amino-3-(5-bromo-thiophen-2-yl)-1-[4-(2,5-difluoro-benzoyl)-piperazin-1-yl]-propan-1-one;
2-Amino-1-[4-(2,5-difluoro-benzoyl)-piperazin-1-yl]-3-(3-trifluoromethyl-phenyl)-propan-1-one;
2-Amino-1-(4-benzhydryl-piperazin-1-yl)-3-[5-(2,6-dichloro-phenyl)-thiophen-2-yl]-propan-1-one;
2-Amino-1-[4-benzhydryl-piperazin-1-yl]-3-[2,2′]bithiophenyl-5-yl-propan-1-one;
2-Amino-1-[4-(3-bromo-phenyl)-piperazin-1-yl]-3-(2,4-dichloro-phenyl)-propan-1-one;
2-Amino-1-[4-(5-chloro-2-methoxy-phenyl)-piperazin-1-yl]-3-[5-(2,4-dichloro-phenyl)-thiophen-2-yl]-propan-1-one;
2-Amino-1-(4-benzhydryl-piperazin-1-yl)-3-[5-(2,4-difluoro-phenyl)-thiophen-2-yl]-propan-1-one;
2-Amino-3-(5-bromo-thiophen-2-yl)-1-(4-methyl-piperazin-1-yl)-propan-1-one;
2-Amino-3-(2,4-dichloro-phenyl)-1-[4-(3,4-dichloro-phenyl)-piperazin-1-yl]-propan-1-one;
2-Amino-1-[4-(3,4-dichloro-phenyl)-piperazin-1-yl]-3-thiophen-2-yl-propan-1-one;
2-Amino-1-(4-pyridin-2-ylmethyl-piperazin-1-yl)-3-thiophen-2-yl-propan-1-one;
2-Amino-3-(5-bromo-thiophen-2-yl)-1-[4-(3,4-dichloro-phenyl)-piperazin-1-yl]-propan-1-one;
2-Amino-1-[4-(3′-chloro-biphenyl-3-yl)-piperazin-1-yl]-3-(2,4-dichloro-phenyl)-propan-1-one;
2-Amino-1-[4-(2,4-dichloro-5-fluoro-pyridine-3-carbonyl)-piperazin-1-yl]-3-(2,4-dichloro-phenyl)-propan-1-one;
2-Amino-3-(3-chloro-phenyl)-1-[4-(2,4-dichloro-5-fluoro-pyridine-3-carbonyl)-piperazin-1-yl]-propan-1-one;
2-Amino-1-[4-(2,4-dichloro-5-fluoro-pyridine-3-carbonyl)-piperazin-1-yl]-3-(2-trifluoromethyl-phenyl)-propan-1-one;
2-Amino-1-[4-(2,4-dichloro-5-fluoro-pyridine-3-carbonyl)-piperazin-1-yl]-3-thiophen-2-yl-propan-1-one;
2-Amino-3-(5-bromo-thiophen-2-yl)-1-[4-(2,4-dichloro-5-fluoro-pyridine-3-carbonyl)-piperazin-1-yl]-propan-1-one;
2-Amino-1-[4-(2,4-dichloro-5-fluoro-pyridine-3-carbonyl)-piperazin-1-yl]-3-(3-trifluoromethyl-phenyl)-propan-1-one;
2-Amino-3-(5-bromo-thiophen-2-yl)-1-(4-pyridin-2-ylmethyl-piperazin-1-yl)-propan-1-one;
2-Amino-1-(4-methyl-piperazin-1-yl)-3-thiophen-2-yl-propan-1-one;
2-Amino-1-(4-biphenyl-3-yl-piperazin-1-yl)-3-(2,4-dichloro-phenyl)-propan-1-one;
2-Amino-1-(4-benzhydryl-piperazin-1-yl)-3-[5-(2-trifluoromethoxy-phenyl)-thiophen-2-yl]-propan-1-one;
2-Amino-1-(4-benzhydryl-piperazin-1-yl)-3-[5-(5-fluoro-2-methyl-phenyl)-thiophen-2-yl]-propan-1-one;
2-Amino-1-(4-benzhydryl-piperazin-1-yl)-3-[5-(2-chloro-4-trifluoromethyl-phenyl)-thiophen-2-yl]-propan-1-one;
2-Amino-1-[4-(5-chloro-2-methyl-phenyl)-piperazin-1-yl]-3-[5-(2,4-dichloro-phenyl)-thiophen-2-yl]-propan-1-one;
2-Amino-3-(2-chloro-phenyl)-1-[4-(2,4-dichloro-5-fluoro-pyridine-3-carbonyl)-piperazin-1-yl]-propan-1-one;
2-Amino-1-(4-benzhydryl-piperazin-1-yl)-3-[5-(4-fluoro-2-methyl-phenyl)-thiophen-2-yl]-propan-1-one;
2-Amino-3-[5-(2,4-dichloro-phenyl)-thiophen-2-yl]-1-(4-indan-2-yl-piperazin-1-yl)-propan-1-one;
2-Amino-3-(2,4-dichloro-phenyl)-1-[4-(3-pyridin-3-yl-phenyl)-piperazin-1-yl]-propan-1-one;
2-Amino-1-(4-benzhydryl-piperazin-1-yl)-3-[5-(2,4-bis-trifluoromethyl-phenyl)-thiophen-2-yl]-propan-1-one;
2-Amino-3-(5-bromo-thiophen-2-yl)-1-(4-pyridin-4-yl-methyl-piperazin-1-yl)-propan-1-one;
2-Amino-3-[5-(2,4-dichloro-phenyl)-thiophen-2-yl]-1-(4-methyl-piperazin-1-yl)-propan-1-one;
2-Amino-3-(5-bromo-thiophen-2-yl)-1-[4-(4-methoxy-benzyl)-piperazin-1-yl]-propan-1-one;
2-Amino-3-(2-chloro-phenyl)-1-[4-(6-chloro-pyridin-2-yl)-piperazin-1-yl]-propan-1-one;
2-Amino-3-(3-chloro-phenyl)-1-[4-(6-chloro-pyridin-2-yl)-piperazin-1-yl]-propan-1-one;
2-Amino-3-(5-bromo-thiophen-2-yl)-1-[4-(6-chloro-pyridin-2-yl)-piperazin-1-yl]-propan-1-one;
2-Amino-1-[4-(6-chloro-pyridin-2-yl)-piperazin-1-yl]-3-[5-(2,4-dichloro-phenyl)-thiophen-2-yl]-propan-1-one;
2-Amino-3-[5-(2,4-dichloro-phenyl)-thiophen-2-yl]-1-(4-pyridin-2-ylmethyl-piperazin-1-yl)-propan-1-one;
2-Amino-1-(4-benzhydryl-piperazin-1-yl)-3-pyridin-3-yl-propan-1-one;
2-Amino-1-(4-benzhydryl-piperazin-1-yl)-3-(5-pyridin-3-yl-thiophen-2-yl)-propan-1-one;
2-Amino-1-(4-benzhydryl-piperazin-1-yl)-3-(5-phenyl-thiophen-2-yl)-propan-1-one;
2-Amino-3-[5-(2,4-dichloro-phenyl)-thiophen-2-yl]-1-[4-(4-methoxy-benzyl)-piperazin-1-yl]-propan-1-one;
2-Amino-3-[5-(2,4-dichloro-phenyl)-thiophen-2-yl]-1-(4-pyridin-4-ylmethyl-piperazin-1-yl)-propan-1-one;
2-Amino-1-[4-(2,5-difluoro-benzoyl)-piperazin-1-yl]-3-pyridin-3-yl-propan-1-one;
2-Amino-3-pyridin-3-yl-1-(4-pyridin-4-ylmethyl-piperazin-1-yl)-propan-1-one;
2-Amino-1-[4-(pyridine-3-carbonyl)-piperazin-1-yl]-3-(3-trifluoromethyl-phenyl)-propan-1-one;
2-Amino-1-[4-(5-chloro-2-trifluoromethyl-benzoyl)-piperazin-1-yl]-3-(3-trifluoromethyl-phenyl)-propan-1-one;
2-Amino-1-[4-(5-fluoro-2-trifluoromethyl-benzoyl)-piperazin-1-yl]-3-(3-trifluoromethyl-phenyl)-propan-1-one;
2-Amino-1-(4-methyl-piperazin-1-yl)-3-[5-(2-methyl-4-propoxy-phenyl)-thiophen-2-yl]-propan-1-one;
2-Amino-1-(4-pyridin-4-ylmethyl-piperazin-1-yl)-3-(3-trifluoromethyl-phenyl)-propan-1-one;
2-Amino-1-(4-benzoyl-piperazin-1-yl)-3-[5-(2,4-dichloro-phenyl)-thiophen-2-yl]-propan-1-one;
2-Amino-3-(3-chloro-phenyl)-1-(4-pyridin-4-ylmethyl-piperazin-1-yl)-propan-1-one;
2-Amino-3-[5-(4-chloro-2-trifluoromethyl-phenyl)-thiophen-2-yl]-1-(4-methyl-piperazin-1-yl)-propan-1-one;
2-Amino-1-(4-pyridin-4-ylmethyl-piperazin-1-yl)-3-(2-trifluoromethyl-phenyl)-propan-1-one;
2-Amino-1-(4-pyridin-4-ylmethyl-piperazin-1-yl)-3-m-tolyl-propan-1-one;
2-Amino-1-(4-benzhydryl-piperazin-1-yl)-3-(4-bromo-phenyl)-propan-1-one;
2-Amino-1-(4-benzhydryl-piperazin-1-yl)-3-(2′,4′-dichloro-biphenyl-4-yl)-propan-1-one;
2-Amino-3-(4-amino-phenyl)-1-[4-(6-chloro-pyridin-2-yl)-piperazin-1-yl]-propan-1-one;
(4-{2-Amino-3-[4-(6-chloro-pyridin-2-yl)-piperazin-1-yl]-3-oxo-propyl}-phenylamino)-acetic acid ethyl ester;
N-{2-[2-Amino-3-(2,4-dichloro-phenyl)-propionyl]-3-dihydro-1H-isoindol-5-yl}-benzamide;
N-{2-[2-Amino-3-(3-chloro-phenyl)-propionyl]-2,3-dihydro-1H-isoindol-5-yl}-benzamide;
N-{2-[2-Amino-3-(5-bromo-thiophen-2-yl)-propionyl]-2,3-dihydro-1H-isoindol-5-yl}-benzamide;
N-{2-[2-Amino-3-(2,4-dichloro-phenyl)-propionyl]-2,3-dihydro-1H-isoindol-5-yl}-2,5-difluoro-benzamide;
2-Amino-1-(4-biphenyl-3-yl-piperidin-1-yl)-3-(3,4-dichloro-phenyl)-propan-1-one;
Thioacetic acid {1-benzyl-2-[4-(5-chloro-2-methyl-phenyl)-piperazin-1-yl]-2-oxo-ethyl}ester;
1-(4-Benzhydryl-piperazin-1-yl)-2-mercapto-3-phenyl-propan-1-one;
2-Amino-3-benzothiazol-2-yl-1-[4-(2,5-difluoro-benzoyl)-piperazin-1-yl]-propan-1-one;
2-Amino-3-benzo[b]thiophen-3-yl-1-[4-(2,5-difluoro-benzoyl)-piperazin-1-yl]-propan-1-one;
2-Amino-1-[4-(2,5-difluoro-benzoyl)-piperazin-1-yl]-2-thiophen-3-yl-ethanone;
2-Amino-3-benzothiazol-2-yl-1-[4-(pyridine-3-carbonyl)-piperazin-1-yl]-propan-1-one;
2-Amino-3-benzo[b]thiophen-3-yl-1-[4-(pyridine-3-carbonyl)-piperazin-1-yl]-propan-1-one;
2-Amino 1-[4-(pyridine-3-carbonyl)-piperazin-1-yl]-2-thiophen-3-yl-ethanone;
2-Amino-1-[4-(5-chloro-2-methyl-phenyl)-piperazin-1-yl]-3-(2,4-dichloro-phenyl)-propan-1-one;
2-Amino-1-[4-(5-chloro-2-methyl-phenyl)-piperazin-1-yl]-3-(3-chloro-phenyl)-propan-1-one;
2-Amino-1-(4-benzhydryl-piperazin-1-yl)-3-(2,4-dichloro-phenyl)-propan-1-one;
2-Amino-1-(4-benzhydryl-piperazin-1-yl)-3-(3-chloro-phenyl)-propan-1-one; and
2-Amino-1-(4-benzoyl-piperazin-1-yl)-3-(3-chloro-phenyl)-propan-1-one;
2-Amino-1-(1,3-dihydro-isoindol-2-yl)-3-(4-propyl-phenyl)-propan-1-one
(E)-(R)-2-Amino-1-(1,3-dihydro-isoindol-2-yl)-5-phenyl-pent-4-en-1-one
(R)-2-Amino-3-biphenyl-4-yl-1-(1,3-dihydro-isoindol-2-yl)-propan-1-one
3-{4-[(R)-2-Amino-3-(1,3-dihydro-isoindol-2-yl)-3-oxo-propyl]-phenyl}-propionic acid methyl ester
(R)-2-Amino-1-(1,3-dihydro-isoindol-2-yl)-3-(4-hydroxy-phenyl)-propan-1-one
(R)-2-Amino-3-cyclohexyl-1-(1,3-dihydro-isoindol-2-yl)-propan-1-one
(R)-2-Amino-1-(1,3-dihydro-isoindol-2-yl)-4-methylsulfanyl-butan-1-one
(R)-2-Amino-1-(1,3-dihydro-isoindol-2-yl)-4-methylsulfanyl-butan-1-one
(R)-2-Amino-1-(1,3-dihydro-isoindol-2-yl)-4-methylsulfanyl-butan-1-one
(S)-2-Amino-3-(4-chloro-phenyl)-1-(1,3-dihydro-isoindol-2-yl)-propan-1-one
(R)-2-Amino-1-(1,3-dihydro-isoindol-2-yl)-3-(4-nitro-phenyl)-propan-1-one
(R)-2-Amino-3-(3,5-difluoro-phenyl)-1-(1,3-dihydro-isoindol-2-yl)-propan-1-one
(R)-2-Amino-3-(4-benzyloxy-phenyl)-1-(1,3-dihydro-isoindol-2-yl)-propan-1-one
(R)-4-Amino-5-(1,3-dihydro-isoindol-2-yl)-5-oxo-pentanoic acid 2-Amino-1-(1,3-dihydro-isoindol-2-yl)-3-o-tolyl-propan-1-one
(R)-4-Amino-5-(1,3-dihydro-isoindol-2-yl)-5-oxo-pentanoic acid benzyl ester
(E)-3-{4-[(h)-2-Amino-3-(1,3-dihydro-isoindol-2-yl)-3-oxo-propyl]-phenyl}-acrylic-acid methyl ester
{4-[(R)-2-Amino-3-(1,3-dihydro-isoindol-2-yl)-3-oxo-propyl]-phenoxy}-acetic acid methyl ester
2-Amino-2-biphenyl-4-yl-1-(1,3-dihydro-isoindol-2-yl)-ethanone
(R)-3-Amino-4-(1,3-dihydro-isoindol-2-yl)-4-oxo-butyric acid benzyl ester
(R)-2-Amino-1-(1,3-dihydro-isoindol-2-yl)-4-(4-methyl-benzylsulfanyl)-butan-1-one
(R)-2-Amino-1-(1,3-dihydro-isoindol-2-yl)-2-(4-fluoro-phenyl)-ethanone 2-Amino-1-(1,3-dihydro-isoindol-2-yl)-3-(2,4-dimethyl-phenyl)-propan-1-one
(R)-4-Amino-5-(1,3-dihydro-isoindol-2-yl)-5-oxopentanoic acid cyclohexyl ester
(R)-7-Amino-1-(1,3-dihydro-isoindol-2-yl)-3-(2-fluoro-phenyl)-propan-1-one
(R)-2-Amino-1-(1,3-dihydro-isoindol-2-yl)-1-[4-(2,3-dihydroxy-propyl)-phenyl]-propan-1-one
(R)-3-(4-Allyloxy-phenyl)-2-amino-1-(1,3-dihydro-isoindol-2-yl)-propan-1-one
(E)-3-{4-[(R)-2-Amino-3-(1,3-dihydro-isoindol-2-yl)-3-oxo-propyl]-phenyl}-acrylic acid
(R)-2-Amino-1-(1,3-dihydro-isoindol-2-yl)-3-(3-nitro-phenyl)-propan-1-one
4′-[(R)-2-Amino-3-(1,3-dihydro-isoindol-2-yl)-3-oxo-propyl]-biphenyl-4-carboxylic acid
(R)-2-Amino-3-(3-chloro-phenyl)-1-(1,3-dihydro-isoindol-2-yl)-propan-1-one
(R)-4-Amino-5-(1,3-dihydro-isoindol-2-yl)-5-oxo-pentanoic acid allyl ester
4′-[(R)-2-Amino-3-(1,3-dihydro-isoindol-2-yl)-3-oxo-propyl]-biphenyl-3-carboxylic acid
(2R,3S)-2-Amino-1-(1,3-dihydro-isoindol-2-yl)-3-hydroxy-butan-1-one
2-Amino-1-(1,3-dihydro-isoindol-2-yl)-3-(4-methoxy-2-methyl-phenyl)-propan-1-one
(R)-2-Amino-3-(3,4-dichloro-phenyl)-1-(1,3-dihydro-isoindol-2-yl)-propan-1-one
2-Amino-3-(4-chloro-2-methoxy-phenyl)-1-(1,3-dihydro-isoindol-2-yl)-propan-1-one
2-Amino-3-[2-chloro-4-(5-phenyl-pent-1-ynyl)-phenyl]-1-(1,3-dihydro-isoindol-2-yl)-propan-1-one
(R)-2-Amino-3-(2,4-dichloro-phenyl)-1-[5-(2-p-tolyl-ethyl)-1,3-dihydro-isoindol-2-yl]-propan-1-one
(R)-2-Amino-3-(2,4-dichloro-phenyl)-1-[5-((E)-2-p-tolyl-vinyl)-1,3-dihydro-isoindol-2-yl]-propan-1-one
(R)-2-Amino-1-[5-(benzhydryl-amino)-1,3-dihydro-isoindol-2-yl]-3-(2,4-dichloro-phenyl)-propan-1-one
2-Amino-3-(2-chloro-4-thiophen-2-yl-phenyl)-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-2-(1H-indol-3-yl)-acetamide
2-Amino-3-(1-benzenesulfonyl-1H-indol-2-yl)-1-(1,3-dihydro-isoindol-2-yl)-propan-1-one
2-Amino-3-(3-chloro-biphenyl-4-yl)-1-(1,3-dihydro-isoindol-2-yl)-propan-1-one
2-Amino-3-(2-chloro-4-methoxy-phenyl)-1-(1,3-dihydro-isoindol-2-yl)-propan-1-one
3-(5-Allyl-naphthalen-1-yl)-2-amino-1-(1,3-dihydro-isoindol-2-yl)-propan-1-one
2-Amino-1-(1,3-dihydro-isoindol-2-yl)-3-naphthalen-1-yl-propan-1-one
(R)-3-Amino-4-(1,3-dihydro-isoindol-2-yl)-4-oxo-butyric acid allyl ester
(R)-3-Amino-4-(1,3-dihydro-isoindol-2-yl)-4-oxo-butyric acid cyclohexyl ester
(R)-2-Amino-1-(1,3-dihydro-isoindol-2-yl)-3-pyrid-in-2-yl-propan-1-one
2-Amino-1-(1,3-dihydro-isoindol-2-yl)-3-(5-phenyl-naphthalen-1-yl)-propan-1-one
2-Amino-1-(1,3-dihydro-isoindol-2-yl)-3-(4-phenyl-naphthalen-1-yl)-propan-1-one
(R)-2-Amino-3-(2,4-dichloro-phenyl)-1-(1,3-dihydro-isoindol-2-yl)-propan-1-one
4-[2-Amino-3-(3-dihydro-isoindol-2-yl)-oxo-propyl]-3-chloro-benzonitrile
Acetic acid 4-[2-amino-3-(1,3-dihydro-isoindol-2-yl)-3-oxo-propyl]-3-chloro-phenyl ester
2-Amino-3-(3-chloro-3′-methyl-biphenyl-4-yl)-1-(1,3-dihydro-isoindol-2-yl)-propan-1-one
(R)-2-Amino-3-(5-bromo-2-methoxy-phenyl)-1-(1,3-dihydro-isoindol-2-yl)-propan-1-one
2-Amino-3-[2-chloro-4-((E)-styryl)-phenyl]-1-(1,3-dihydro-isoindol-2-yl)-propan-1-one
(R)-2-Amino-3-[4-(2,6-dichloro-benzyloxy)-phenyl]-1-(1,3-dihydro-isoindol-2-yl)-propan-1-one
2-Amino-3-[2-chloro-4-(4-phenyl-but-1-ynyl)-phenyl]-1-(1,3-dihydro-isoindol-2-yl)-propan-1-one
2-Amino-3-[2-chloro-4-(2-methyl-propenyl)-phenyl]-1-(1,3-dihydro-isoindol-2-yl)-propan-1-one
2-Amino-3-(3,3′-dichloro-biphenyl-4-yl)-1-(1,3-dihydro-isoindol-2-yl)-propan-1-one
5-[2-Amino-3-(1,3-dihydro-isoindol-2-yl)-3-oxo-propyl]-naphthalene-1-carbonitrile
2-Amino-3-{2-chloro-4-[(E)-2-(4-chloro-phenyl)-vinyl]-phenyl}-1-(1,3-dihydro-isoindol-2-yl)-propan-1-one
2-Amino-3-{2-chloro-4-[(E)-2-(4-methoxy-phenyl)-vinyl]-phenyl}-1,3-dihydro-isoindol-2-yl)-propan-1-one
2-Amino-3-[2-chloro-4-((E)-2-p-tolyl-vinyl)-phenyl]-1-(1,3-dihydro-isoindol-2-yl)-propan-1-one
2-Amino-3-(2,3-dichloro-phenyl)-1-(1,3-dihydro-isoindol-2-yl)-propan-1-one
2-Amino-3-(4-bromo-2-chloro-phenyl)-1-(1,3-dihydro-isoindol-2-yl)-propan-1-one
2-Amino-3-(3-chloro-2′-methyl-biphenyl-4-yl)-1-(1,3-dihydro-isoindol-2-yl)-propan-1-one
2-Amino-3-[2-chloro-4-(3-phenoxy-prop-1-ynyl)-phenyl]-1-(1,3-dihydro-isoindol-2-yl)-propan-1-one
2-Amino-3-(2,4-dichloro-3-methyl-phenyl)-1-(1,3-dihydro-isoindol-2-yl)-propan-1-one
4′-[2-Amino-3-(3-dihydro-isoindol-2-yl)-3-oxo-propyl]-3′-chloro-biphenyl-3-carbonitrile
2-Amino-3-(3-chloro-4′-isopropyl-biphenyl-4-yl)-1-(1,3-dihydro-isoindol-2-yl)-propan-1-one
2-Amino-3-(3-chloro-2′-methoxy-biphenyl-4-yl)-1-(1,3-dihydro-isoindol-2-yl)-propan-1-one
2-Amino-3-(3-chloro-2′-fluoro-biphenyl-4-yl)-1-(1,3-dihydro-isoindol-2-yl)-propan-1-one
2-Amino-1-(1,3-dihydro-isoindol-2-yl)-3-(3,2′,4′-trichloro-biphenyl-4-yl)-propan-1-one
2-Amino-3-(2-chloro-4-phenylethynyl-phenyl)-1-(1,3-dihydro-isoindol-2-yl)-propan-1-one
2-Amino-3-(2-chloro-4-methyl-phenyl)-1-(1,3-dihydro-isoindol-2-yl)-propane-one
2-Amino-3-(2-chloro-4-hydroxy-phenyl)-1-(1,3-dihydro-isoindol-2-yl)-propan-1-one
2-Amino-3-(3,4′-dichloro-biphenyl-4-yl)-1-(1,3-dihydro-isoindol-2-yl)-propan-1-one
2-Amino-3-(3-chloro-4′-methoxy-biphenyl-4-yl)-1-(1,3-dihydro-isoindol-2-yl)-propan-1-one
2-Amino-3-(3-chloro-4′-methyl-biphenyl-4-yl)-1-(1,3-dihydro-isoindol-2-yl)-propan-1-one
(R)-2-Amino-1-[5-(2-chloro-phenyl)-1,3-dihydro-isoindol-2-yl]-3-(2,4-dichloro-phenyl)-propan-1-one
2-Amino-3-(6-chloro-benzo[1,3]dioxol-5-yl)-1-(1,3-dihydro-isoindol-2-yl)-propan-1-one
(R)-2-Amino-1-[5-(2,4-dichloro-benzylamino)-1,3-dihydro-isoindol-2-yl]-3-(2,4-dichloro phenyl)-propan-1-one
(R)-2-Amino-3-(2,4-dichloro-phenyl)-1-{5-[(pyridin-4-ylmethyl)-amino]-1,3-dihydro-isoindol-2-yl}-propan-1-one
1-{2-[(R)-2,4-Amino-3-(2,4-dichloro-phenyl)-propionyl]-2,3-dihydro-1H-isoindol-5-yl}-3-(3,5-dichloro-phenyl)-urea
1-{2-[(R)-2-Amino-3-(2,4-dichloro-phenyl)-propionyl]-2,3-dihydro-1H-isoindol-5-yl}-3-isopropyl-urea
1-{2-[(R)-2-Amino-3-(2,4-dichloro-phenyl)-propionyl]-2,3-dihydro-1H-isoindol-5-ylmethyl}-3-(4-dimethylamino-phenyl)-urea
1-{2-[(R)-2-Amino-3-(2,4-dichloro-phenyl)-propionyl]-2,3-dihydro-1H-isoindol-5-yl}-3-benzyl-urea
(R)-2-Amino-3-(2,4-dichloro-phenyl)-1-(5-naphthalen-1-yl-1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-acetamide
(R)-2-Amino-3-(2,4-dichloro-phenyl)-1-[5-(4-methoxy-benzylamino)-1,3-dihydro-isoindol-2-yl]-propan-1-one
N-{4-[2-Amino-3-(1,3-dihydro-isoindol-2-yl)-3-oxo-propyl]-3-chloro-phenyl}-methanesulfonamide
N-{2-[(R)-2-Amino-3-(2,4-dichloro-phenyl)-propionyl]-2,3-dihydro-1H-isoindol-5-yl}-propionamide
1-{2-[(R)-2-Amino-3-(2,4-dichloro-phenyl)-propionyl]-2,3-dihydro-1H-isoindol-5-yl}-3-phenyl-urea
1-{2-[(R)-2-Amino-3-(2,4-dichloro-phenyl)-propionyl]-2,3-dihydro-1H-isoindol-1-yl}-3-(3-benzyl-phenyl)-urea
(E)-3-{5-[(R)-2-Amino-3-(1,3-dihydro-isoindol-2-yl)-3-oxo-propyl]-thiophen-2-yl}-acrylic acid methyl ester
(E)-4-{5-[(R)-2-Amino-3-(1,3-dihydro-isoindol-2-yl)-3-oxo-propyl]-thiophen-2-yl}-but-2-enoic acid methyl ester
(R)-2-Amino-1-[5-(cyclohexylmethyl-amino)-1,3-dihydro-isoindol-2-yl]-3-(2,4-dichloro-phenyl)-propan-1-one
1-{2-[(R)-2-Amino-3-(2,4-dichloro-phenyl)-propionyl]-2,3-dihydro-1H-isoindol-5-yl}-3-(3-phenoxy-phenyl)-urea
1-{2-[(R)-2-Amino-3-(2,4-dichloro-phenyl)-propionyl]-2,3-dihydro-isoindol-5-yl}-3-(4′-methyl-biphenyl-4-yl)-urea
(R)-2-Amino-1-(5,6-dichloro-1,3-dihydro-isoindol-2-yl)-3-(2,4-dichloro-phenyl)-propan-1-one
(R)-2-Amino-1-[5-(3-chloro-phenyl)-1,3-dihydro-isoindol-2-yl]-3-(2,4-dichloro-phenyl)-propan-1-one
(R)-2-Amino-3-(2,4-dichloro-phenyl)-1-[5-(3-methoxy-phenyl)-1,3-dihydro-isoindol-2-yl]-propan-1-one
4-{5-[(R)-2-Amino-3-(1,3-dihydro-isoindol-2-yl)-3-oxo-propyl]-thiophen-2-yl}-benzoic acid methyl ester
3-Methyl-but-2-enoic acid {2-[(R)-2-amino-1-(2,4-dichloro-phenyl)-propionyl]-2,3-dihydro-1H-isoindol-5-yl}-amide
1-{2-[(R)-2-Amino-3-(2,4-dichloro-phenyl)-propionyl]-2,3-dihydro-1H-isoindol-5-yl}-3-(2,4-dichloro-phenyl)-urea
N-{2-[(R)-2-Amino-3-(3-chloro-phenyl)-propionyl]-2,3-dihydro-1H-isoindol-5-yl}-benzamide
2-Amino-3-(2-chloro-4-methanesulfonyl-phenyl)-1-(1,3-dihydro-isoindol-2-yl)-propan-1-one
3-(4-Allyl-2-chloro-phenyl)-2-amino-1-(1,3-dihydro-isoindol-2-yl)-propan-1-one
2-Amino-1-(1,3-dihydro-isoindol-2-yl)-3-quinolin-7-yl-propan-1-one
2-Amino-3-(3,2′-dichloro-biphenyl-4-yl)-1-(1,3-dihydro-isoindol-2-yl)-propan-1-one
2-Amino-3-(3-chloro-3′-methoxy-biphenyl-4-yl)-1-(1,3-dihydro-isoindol-2-yl)-propan-1-one
2-Amino-3-(4-benzyloxy-2-chloro-phenyl)-1-(1,3-dihydro-isoindol-2-yl)-propan-1-one
2-Amino-3-{2-chloro-4-[(E)-2-(3-fluoro-phenyl)-vinyl]-phenyl}-1-(1,3-dihydro-isoindol-2-yl)-propan-1-one
2-Amino-3-{2-chloro-4-[(E)-2-(4-fluoro-phenyl)-vinyl]-phenyl}-1-(1,3-dihydro-isoindol-2-yl)-propan-1-one
2-Amino-3-(2-chloro-3,4-dimethoxy-phenyl)-1-(1,3-dihydro-isoindol-2-yl)-propan-1-one
2-Amino-3-[2-chloro-4-((E)-2-cyclohexyl-vinyl phenyl]-1-(1,3-dihydro-isoindol-2-yl)-propan-1-one
2-Amino-3-(2,4-dichloro-6-hydroxy-phenyl)-1-(1,3-dihydro-isoindol-2-yl)-propan-1-one
2-Amino-3-(3-chloro-4′-methoxy-3′-methyl-biphenyl-4-yl)-1-(1,3-dihydro-isoindol-2-yl)-propan-1-one
(R)-2-Amino-3-(2,4-dichloro-phenyl)-1-[5-((E)-styryl)-1,3-dihydro-isoindol-2-yl]-propan-1-one
2-Amino-3-(2,4-dichloro-5-fluoro-phenyl)-1-(1,3-dihydro-isoindol-2-yl)-propan-1-one
2-Amino-3-(1-chloro-naphthalen-2-yl)-1-(1,3-dihydro-isoindol-2-yl)-propan-1-one
2-Amino-3-(3-chloro-2′,5′-dimethoxy-biphenyl-4-yl)-1-(1,3-dihydro-isoindol-2-yl)-propan-1-one
2-Amino-3-(3-bromo-2,4-dichlorophenyl)-1-(1,3-dihydro-isoindol-2-yl)-propan-1-one
(R)-2-Amino-1-(5-benzylamino-1,3-dihydro-isoindol-2-yl)-3-(2,4-dichloro-phenyl)-propan-1-one
2-Amino-3-(2,4-dichloro-6-methoxy-phenyl)-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-ylmethyl}-isobutyramide
(S)-2-Amino-N-{-[(R)-2-amino-3-(2,4-dichloro-phenyl)-propionyl]-2,3-dihydro-1H-isoindol-5-yl}-3-(2,4-dichloro-phenyl)-propionamide
1-{2-[(R)-2-Amino-3-(2,4-dichloro-phenyl)-propionyl]-2,3-dihydro-1H-isoindol-5-yl}-3-p-tolyl-urea
1-{2-[(R)-2-Amino-3-(2,4-dichloro-phenyl)-propionyl]-2,3-dihydro-1H-isoindol-5-yl}-3-(4-phenoxy-phenyl)-urea
1-{2-[(R)-2-Amino-3-(2,4-dichloro-phenyl)-propionyl]-2,3-dihydro-1H-isoindol-5-yl}-3-biphenyl-4-yl-urea
N-{2-[(R)-2-Amino-3-(2,4-dichloro-phenyl)-propionyl]-2,3-dihydro-1H-isoindol-5-yl}-isobutyramide
({2-[(R)-2-Amino-3-(2,4-dichloro-phenyl)-propionyl]-2,3-dihydro-1H-isoindol-5-yl}-methoxyoxalyl-amino)-oxo-acetic acid methyl ester
1-{2-[(R)-2-Amino-3-(2,4-dichloro-phenyl)-propionyl]-2,3-dihydro-1H-isoindol-5-yl}-3-(4-dimethylamino-phenyl)-urea
2-[(R)-2-Amino-3-(2,4-dichloro-phenyl)-propionyl]-2,3-dihydro-1H-isoindole-5-carbonitrile
(R)-2-Amino-1-(5-aminomethyl-1,3-dihydro-isoindol-2-yl)-3-(2,4-dichloro-phenyl)-propan-1-one
N-{2-[(R)-2-Amino-3-(2,4-dichloro-phenyl)-propionyl]-2,3-dihydro-1H-isoindol-5-ylmethyl}-acetamide
2-Amino-3-(2,5-dichloro-phenyl)-1-(1,3-dihydro-isoindol-2-yl)-propan-1-one
3-(4′-Acetyl-3-chloro-biphenyl-4-yl)-2-amino-1-(1,3-dihydro-isoindol-2-yl)-propan-1-one
4′-[2-Amino-3-(1,3-dihydro-isoindol-2-yl)-3-oxo-propyl]-3′-chloro-biphenyl-4-carbonitrile
2-Amino-3-(5-bromo-naphthalen-1-yl)-1-(1,3-dihydro-isoindol-2-yl)-propan-1-one
2-Amino-3-(4-bromo-naphthalen-1-yl)-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-oxalamic acid methyl ester
(R)-2-Amino-3-(2,4-dichloro-phenyl)-1-{5-[(pyridin-3-ylmethyl)-amino]-1,3-dihydro-isoindol-2-yl}-propan-1-one
1-{2-[(R)-2-Amino-3-(2,4-dichloro-phenyl)-propionyl]-2,3-dihydro-1H-isoindol-5-yl}-3-(3,5-dimethoxy-phenyl)-urea
(S)-2-Acetylamino-N-{2-[(R)-2-amino-3-(2,4-dichloro-phenyl)-propionyl]-2,3-dihydro-1H-isoindol-5-yl}-3-(1H-indol-3-yl)-propionamide
2-Amino-1-(1,3-dihydro-isoindol-2-yl)-3-(1H-indol-6-yl)-propan-1-one
(R)-2-Amino-3-[5-(2-chloro-phenyl)-thiophen-2-yl]-1-(1,3-dihydro-isoindol-2-yl)-propan-1-one
(R)-2-Amino-1-(5-bromo-1,3-dihydro-isoindol-2-yl)-3-(2,4-dichloro-phenyl)-propan-1-one
(R)-2-Amino-3-(2,4-dichloro-phenyl)-1-(5-nitro-1,3-dihydro-isoindol-2-yl)-propan-1-one
(R)-2-Amino-3-(2,4-dichlorophenyl)-1-(5-fluoro-1,3-dihydro-isoindol-2-yl)-propan-1-one
(R)-2-Amino-3-(2,4-dichloro-phenyl)-1-(5-methyl-1,3-dihydro-isoindol-2-yl)-propan-1-one
3-{2-[(R)-2-Amino-3-(2,4-dichloro-phenyl)-propionyl]-2,3-dihydro-1H-isoindol-5-yl}-benzonitrile
N-{2-[(R)-2-Amino-3-(2,4-dichloro-phenyl)-propionyl]-2,3-dihydro-1H-isoindol-5-ylmethyl}-2-methyl-butyramide
3-(4-Allyloxy-2-chloro-phenyl)-2-amino-(1,3-dihydro-isoindol-1-yl)-propan-1-one
3-(3′-Acetyl-3-chloro-biphenyl-4-yl)-2-amino-1-(1,3-dihydro-isoindol-2-yl)-propan-1-one
2-Amino-3-(3,3′-dichloro-4′-fluoro-biphenyl-4-yl)-1-(1,3-dihydro-isoindol-2-yl)-propan-1-one
2-Amino-3-[5-(2-chloro-phenyl)-naphthalen-1-yl]-1-(1,3-dihydro-isoindol-2-yl)-propan-1-one
2-Amino-3-[4-(2-chloro-phenyl)-naphthalen-1-yl]-1-(1,3-dihydro-isoindol-2-yl)-propan-1-one
(R)-2-Amino-3-[5-(4-chloro-phenyl)-thiophen-2-yl]-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-2-phenyl-butyramide
N-{2-[(R)-2-Amino-3-(2,4-dichloro-phenyl)-propionyl]-2,3-dihydro-1H-isoindol-5-yl}-3-methyl-butyramide
N-{2-[(R)-2-Amino-3-(2,4-dichloro-phenyl)-propionyl]-2,3-dihydro-1H-isoindol-5-yl}-4-trifluoromethyl-benzamide
3-{2-[(R)-2-Amino-3-(2,4-dichloro-phenyl)-propionyl]-2,3-dihydro-1H-isoindol-5-yl}-N,N-dimethyl-benzamide
(R)-2-Amino-3-(2,4-dichloro-phenyl)-1-(5-naphthalen-2-yl)-1,3-dihydro-isoindol-2-yl)-propan-1-one
(R)-2-Amino-3-(2,4-dichloro-phenyl)-1-[5-(2-methoxy-phenyl)-1,3-dihydro-isoindol-2-yl]-propan-1-one
N-{2-[(R)-2-Amino-3-(2,4-dichloro-phenyl)-propionyl]-2,3-dihydro-1H-isoindol-5-ylmethyl}-oxalamic acid methyl ester
1-{2-[(R)-2-Amino-3-(2,4-dichloro-phenyl)-propionyl]-2,3-dihydro-1H-isoindol-5-ylmethyl}-3-isopropyl-urea
(R)-2-Amino-3-[5-(3-chloro-phenyl)-thiophen-2-yl]-1-(1,3-dihydro-isoindol-2-yl)-propan-1-one
3-{5-[(R)-2-Amino-3-(1,3-dihydro-isoindol-2-yl)-3-oxo-propyl]-thiophen-2-yl}-benzoic acid
N-{2-[(R)-2-Amino-3-(2,4-dichloro-phenyl)-propionyl]-2,3-dihydro-1H-isoindol-5-yl}-2-methyl-butyramide
1-{2-[(R)-2-Amino-3-(2,4-dichloro-phenyl)-propionyl]-2,3-dihydro-1H-isoindol-5-yl}-3-(2,5-dimethoxy-phenyl)-urea
(R)-2-Amino-3-[5-(2,4-dichloro-phenyl)-thiophen-2-yl]-1-(1,3-dihydro-isoindol-2-yl)-propan-1-one
(R)-2-Amino-3-(5-bromo-thiophen-2-yl)-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-benzamide
(E)-3-{5-[(R)-2-Amino-3-(1,3-dihydro-isoindol-2-yl)-3-oxo-propyl]-thiophen-2-yl}-acrylic acid
N-{2-[(R)-2-Amino-3-(2,4-dichloro-phenyl)-propionyl]-2,3-dihydro-1H-isoindol-5-yl}-2-(4-dimethylamino-phenyl)-acetamide
(R)-2-Amino-3-(2,4-dichloro-phenyl)-1-(5-isobutylamino-1,3-dihydro-isoindol-2-yl)-propan-1-one
(R)-2-Amino-1-(5-dimethylsulfonamyl-amido-1,3-dihydro-isoindol-2-yl)-3-(2,4-dichloro phenyl)-propan-1-one
1-{2-[(R)-2-Amino-3-(2,4-dichloro-phenyl)-propionyl]-2,3-dihydro-1H-isoindol-5-yl}-3-(4,5-trimethoxy-phenyl)-urea
2-Amino-3-(2-chloro-4-thiophen-3-ylethynyl-phenyl)-1-(1,3-dihydro-isoindol-2-yl)-propan-1-one
(R)-2-Amino-3-(2,4-dichloro-phenyl)-1-[5-(4-pyridin-4-yl-benzylamino)-1,3-dihydro-isoindol-2-yl]-propan-1-one
2-Amino-3-(2,3-dihydro-1H-indol-6-yl)-1-(1,3-dihydro-isoindol-2-yl)-propan-1-one
3-{5-[(R)-2-Amino-3-(1,3-dihydro-isoindol-2-yl)-3-oxo-propyl]-thiophen-2-yl}-benzoic acid methyl ester
N-{2-[(R)-2-Amino-3-(2,4-dichloro-phenyl)-propionyl]-2,3-dihydro-1H-isoindol-5-yl}-2,5-difluoro-benzamide
1-(4-Acetyl-phenyl)-3-{2-[(R)-2-amino-3-(2,4-dichloro-phenyl)-propionyl]-2,3-dihydro-1H-isoindol-5-yl}-urea
(R)-2-Amino-1-(5-bis-methylsulfon-amido-1,3-dihydro-isoindol-2-yl)-3-(2,4-dichloro-phenyl)-propan-1-one
N-{2-[(R)-2-Amino-3-(2,4-dichloro-phenyl)-propionyl]-2,3-dihydro-1H-isoindol-5-yl}-3,3-dimethyl-butyramide
1-{2-[(R)-2-Amino-3-(2,4-dichloro-phenyl)-propionyl]-2,3-dihydro-1H-isoindol-5-yl}-3,3-bis(3,5-dimethoxy-phenyl)-urea
N-{2-[(R)-2-Amino-3-(2,4-dichloro-phenyl)-propionyl]-2,3-dihydro-1H-isoindol-5-yl}-4-dimethylamino-benzamide
Cyclopentanecarboxylic acid {2-[(R)-2-amino-3-(2,4-dichloro-phenyl)-propionyl]-2,3-dihydro-1H-isoindol-5-yl}-amide
1-{2-[(R)-2-Amino-3-(2,4-dichloro-phenyl)-propionyl]-2,3-dihydro-1H-isoindol-5-yl}-3-benzoyl-urea
(R)-3-(5-Allyl-thiophen-2-yl)-2-amino-1-(1,3-dihydro-isoindol-2-yl)-propan-1-one
(R)-2-Amino-1-(5-amino-1,3-dihydro-isoindol-2-yl)-3-(2,4-dichloro-phenyl)-propan-1-one
(R)-2-Amino-3-[5-(2-bromo-phenyl)-thiophen-2-yl]-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-3-trifluoromethyl-benzamide
Morpholine-4-carboxylic acid {2-[(R)-2-amino-3-(2,4-dichloro-phenyl)-propionyl]-2,3-dihydro-1H-isoindol-5-yl}-amide
2-Amino-3-(4-benzylamino-2-chloro-phenyl)-1-(1,3-dihydro-isoindol-2-yl)-propan-1-one
2-Amino-3-(2-chloro-4-dimethylamino-phenyl)-1-(1,3-dihydro-isoindol-2-yl)-propan-1-one
2-Amino-3-(3-chloro-2′,4′-dimethyl-biphenyl-4-yl)-1-(1,3-dihydro-isoindol-2-yl)-propan-1-one
2-Amino-3-(3-chloro-3′,4′-dimethoxy-biphenyl-4-yl)-1-(1,3-dihydro-isoindol-2-yl)-propan-1-one
4-{5-[(R)-2-Amino-3-(1,3-dihydro-isoindol-2-yl)-3-oxo-propyl]-thiophen-2-yl}-benzoic acid
N-{2-[(R)-2-Amino-3-(2,4-dichloro-phenyl)-propionyl]-2,3-dihydro-1H-1-isoindol-5-yl}-nicotinamide
(R)-2-Amino-3-(2,4-dichloro-phenyl)-1-(1,3-dihydro-benzo[f]isoindol-2-yl)-propan-1-one
N-{2-[(R)-2-Amino-3-(2,4-dichloro-phenyl)-propionyl]-2,3-dihydro-1H-isoindol-5-yl}-2,2,2-trifluoro-acetamide
1-{2-[(R)-Z-Amino-3-(2,4-dichloro-phenyl)-propionyl]-2,3-dihydro-1H-isoindol-5-yl}-3-(S)-1-phenyl-ethyl)-urea
(R)-2-Amino-3-(2,4-dichloro-phenyl)-1-(5-phenyl-1,3-dihydro-isoindol-2-yl)-propan-1-one
(R)-2-Amino-1-(5-biphenyl-3-yl-1,3-dihydro-isoindol-2-yl)-3-(2,4-dichloro-phenyl)-propan-1-one
(R)-2-Amino-1-(1,3-dihydro-isoindol-2-yl)-3-thiophen-2-yl-propan-1-one
2-Amino-3-{2-chloro-4-[(2)-2-(4-trifluoromethyl-phenyl)-vinyl]-phenyl}-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-bis(4-methyl-benzene)-sulfonamide
(R)-2-Amino-3-(5-bromo-thiophen-2-yl)-1-(5-fluoro-1,3-dihydro-isoindol-2-yl)-propan-1-one
2-Amino-1-(1,3-dihydro-isoindol-2-yl)-3-(1H-indol-2-yl)-propan-1-one
(1-Amino-indan-1-yl)-(1,3-dihydro-isoindol-2-yl)-methanone
(R)-2-Amino-1-(5-benzyl-1,3-dihydro-isoindol-2-yl)-3-(2,4-dichloro-phenyl)-propan-1-one
N{2-[(R)-2-Amino-3-(2,4-dichloro-phenyl)-propionyl]-2,3-dihydroxy-1H-isoindol-5-yl}-5-chloro-2-trifluoromethyl-benzamide
N-{2-[(R)-2-Amino-3-(2,4-dichloro-phenyl)-propionyl]-2,3-dihydro-1H-isoindol-5-yl}-methanesulfonamide
N-{2-[(R)-2-Amino-3-(2,4-dichloro-phenyl)-propionyl]-2,3-dihydro-1H-isoindol-5-yl}-5-fluoro-2-trifluoromethyl-benzamide
2-Amino-3-(3-chloro-4′-methoxy-2′-methyl-biphenyl-4-yl)-1-(1,3-dihydro-isoindol-2-yl)-propan-1-one
2-Amino-3-(2-chloro-4-trimethylsilanylethynyl-phenyl)-1-(1,3-dihydro-isoindol-2-yl)-propan-1-one
(R)-2-Amino-1-(1,3-dihydro-isoindol-2-yl)-3-[5-(2-trifluoromethyl-phenyl)-thiophen-2-yl]-propan-1-one
(S)-2-Amino-3-(2,5-dibromo-thiophen-3-yl)-1-(1,3-dihydro-isoindol-2-yl)-propan-1-one
2-Amino-3-[2-chloro-4-(3-methyl-3H-imidazol-4-ylethynyl)-phenyl]-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-4-methoxy-benzene)-sulfonamide
(R)-2-Amino-1-(5-bromo-1,3-dihydro-isoindol-2-yl)-3-(5-bromo-thiophen-2-yl)-propan-1-one
N-{2-[(R)-2-Amino-3-(2,4-dichloro-phenyl)-propionyl]-2,3-dihydro-1H-isoindol-5-yl}-bis(4-methoxy-benzene)sulfonamide
2-Amino-3-(4-benzofuran-2-yl-2-chloro-phenyl)-1-(1,3-dihydro-isoindol-2-yl)-propan-1-one
[(S)-1-{2-[(R)-2-Amino-3-(2,4-dichloro-phenyl)-propionyl]-2,3-dihydro-1H-isoindol-5-ylcarbamoyl}-2-(1H-indol-3-yl)-ethyl]-carbamic acid benzyl ester
(R)-2-Amino-3-benzo[b]thiophen-3-yl-1-(1,3-dihydro-isoindol-2-yl)-propan-1-one
N-{2-[(R)-2-Amino-3-(2,4-dichloro-phenyl)-propionyl]-2,3-dihydro-1H-isoindol-5-yl}-4-methyl-benzenesulfonamide
N-{2-[(R)-2-Amino-3-(5-bromo-thiophen-2-yl)-propionyl]-2,3-dihydro-1H-isoindol-5-yl}-benzamide
(R)-2-Amino-1-(1,3-dihydro-isoindol-2-yl)-propan-1-one
(R)-2-Amino-1-[5-(1H-benzoimidazol-2-ylamino)-1,3-dihydro-isoindol-2-yl]-3-(2,4-dichloro-phenyl)-propan-1-one
(R)-2-Amino-1-[5-(benzooxazol-2-ylamino)-1,3-dihydro-isoindol-2-yl]-3-(2,4-dichloro-phenyl)-propan-1-one
(R)-2-Amino-1-(1,3-dihydro-isoindol-2-yl)-3-methyl-butan-1-one
(R)-2-Amino-3-(2,4-dichloro-phenyl)-1-(2,6-dihydro-4H-pyrrolo[3,4-c]pyrazol-5-yl)-propan-1-one
(R)-2-Amino-3-(5-bromo-thiophen-2-yl)-1-(2,6-dihydro-4H-pyrrolo[3,4-c]pyrazol-5-yl)-propan-1-one
(R)-2-Amino-3-(2,4-dichloro-phenyl)-1-(5,7-dihydro-pyrrolo[3,4-b]pyridin-6-yl)-propan-1-one
(R)-2-Amino-3-(2,4-dichloro-phenyl)-1-(5,7-dihydro-pyrrolo[3,4-d]pyrimidin-yl)-propan-1-one
(R)-2-Amino-3-(5-bromo-thiophen-2-yl)-1-(5,7-dihydro-pyrrolo[3,4-d]pyrimidin-6-yl)-propan-1-one
(R)-2-Amino-3-(5-bromothiophen-2-yl)-1-(5,7-dihydropyrrolo[3,4-b]pyridin-6-yl)-propan-1-one
or pharmaceutically acceptable salts thereof.

9. The compound according to claim 1, wherein the polyheterocycle is selected from a nitrogen-substituted cycloalkyl, aryl or cycloalkaryl, any of which may be further heterosubstituted, and which for example may be selected from a C₃-C₆ cycloalkyl or partially saturated cycloalkyl, C₃-C₆ saturated or partially unsaturated heterocycloalkyl or heterocycloalkenyl (e.g., tetrahydro-pyridine), morpholine, C₃-C₆ heteroaryl, C₃-C₆ polyheteroaryl, C₃-C₆ non-aromatic polyheterocycle, or a fused and/or spiro polyheterocycle selected from decahydro-(iso)quinoline, tetrahydro(iso)quinoline, piperazine, piperidine, indole, (iso)indole, benzyl, furan, or is selected from formula (Ia) through formula (If):

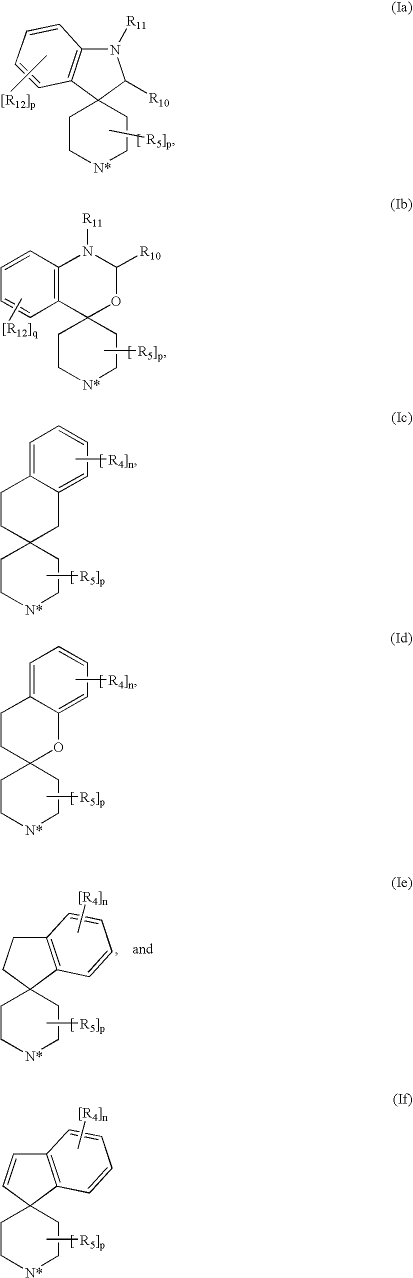

10. A method for treating a disease comprising administering to a mammal in need thereof a compound of Formula I or Formula II, wherein the disease is a proliferative disease, a hyperproliferative disease, a disease of the immune system, or a disease of the central, a disease associated with misexpression of a gene, or peripheral nervous system.

11. The method according to claim 10, wherein the disease is a HDAC dependent disease, wherein the HDAC is selected from the group of HDAC1, HDAC2, HDAC3, HDAC4, HDAC5, HDAC6, HDAC7, HDAC8, HDAC9, HDAC10 and HDAC11, wherein the compound is lacking a hydroxamate or a thio substituent.

12. The method according to claim 11, %% herein the protein HDAC is selected from the group of HDAC1, HDAC2, HDAC6 and HDAC8.

13. The method according to claim 10, wherein the disease to be treated is a proliferative disease, including a hyperproliferative disease, preferably including a benign or especially malignant tumor, more preferably a carcinoma of the brain, kidney, liver, adrenal gland, bladder, breast, stomach (especially gastric tumors), ovaries, esophagus, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia.

14. The method according to claim 10, wherein the disease to be treated is triggered by persistent angiogenesis, such as psoriasis; Kaposi's sarcoma; restenosis, e.g., stent-induced restenosis; endometriosis. Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

15. The method according to claim 10, wherein the disease to be treated is a diseases of the immune system.

16. The method according to claim 10, wherein the hyperproliferative disease is selected from the group of leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

17. A pharmaceutical composition comprising a compound of Formula I or Formula II.

18. A pharmaceutical composition comprising a compound according to claim 17 and a pharmaceutically acceptable carrier or excipient therefor.

19. A kit comprising a compound of Formula I or Formula II.

20. The kit according to claim 19, further comprising a pharmaceutically acceptable carrier or excipient therefor.

21. The kit according to claim 19 wherein the compound is present in a unit dose.

22. The kit according to claim 19, further comprising instructions for use in administering to a subject.

23. A method of selectively inhibiting a histone deacetylase (HDAC), comprising contacting a cell with a compound according to claim 11.

24. The method of claim 23 wherein the compound is present in an amount effective to produce a concentration sufficient to selectively inhibit the acetylation of a histone in the cell.

25-28. (canceled)

29. A method of manufacture of a medicament, the method comprising formulating a compound of Formula I or Formula II for treatment of a subject.

30. The compound according to claim 11 further characterized in that it is an inhibitor of a histone deacetylase.

31. The compound according to claim 2, wherein at least one of R₁, R₂, R₃ is selected from hydrogen.

32. The compound according to claim 2, wherein at least one of R₁, R₂, and R₃ is selected from the group of NHR₆ or NH₂.

33. The compound according to according to claim 29 wherein R₁ is H, and R₂ is NH₂.
